(19) United States
(12) Patent Application Publication
(10) Pub. No.: US 2020/0208276 A1
(43) Pub. Date: Jul. 2, 2020

(54) LOCALIZED EXCESS PROTONS AND ISOTHERMAL ELECTRICITY FOR ENERGY RENEWAL

(71) Applicant: James Weifu Lee, Chesapeake, VA (US)

(72) Inventor: James Weifu Lee, Chesapeake, VA (US)

(21) Appl. No.: 16/237,681

(22) Filed: Jan. 1, 2019

(51) Int. Cl. C25B 1/06; C25B 1/10; C23F 1/14; C12P 19/32; C12P 19/40

(52) U.S. Cl. CPC C25B 1/06; C25B 1/10; C12P 19/40; C12P 19/32; C23F 1/14

(57) ABSTRACT

Inspired by the discovery that environmental heat energy can be isothermally utilized through electrostatically localized protons at a liquid-membrane interface to do useful work such as driving ATP synthesis, the present invention discloses an innovative energy renewal method with making and using an asymmetric function-gated isothermal electricity production system comprising at least one pair of a low work function thermal electron emitter and a high work function electron collector across a barrier space installed in a container with electric conductor support to enable energy recycle process functions with utilization of environmental heat energy isothermally for at least one of: a) utilization of environmental heat energy for energy renewing of fully dissipated waste heat energy from the environment to generate electricity to do useful work; b) providing a novel cooling function for a new type of refrigerator by isothermally extracting environmental heat energy from inside the refrigerator while generating isothermal electricity.

### CROSS-REFERENCE TO RELATED APPLICATIONS

This application is a continuation-in-part of co-pending U.S. patent application Ser. No. 15/202,214 filed on Jul. 5, 2016 that claims priority and benefit from U.S. Provisional Application No. 62/231,402 filed on Jul. 6, 2015. This application also claims priority and benefit from U.S. Provisional Application No. 62/613,912 filed on Jan. 5, 2018. These applications are incorporated herein by reference in their entirety.

### FIELD OF THE INVENTION

The present invention is directed to a series of methods and systems for making and using localized excess protons and more importantly for creating and using asymmetric function-gated isothermal electricity power generator systems to isothermally utilize environmental heat energy to generate electricity to do useful work.

### BACKGROUND

The newly developed proton-electrostatics localization hypothesis in understanding proton-coupling bioenergetics over the Nobel-prize work of Peter Mitchell's chemiosmotic theory (Lee 2012 Bioenergetics 1:104; doi:10.4172/2167-7662.1000104; Lee 2015 Bioenergetics 4: 121. doi:10.4172/2167-7662.1000121) resulted in the following new protonic motive force (pmf) equation that may potentially represent a major breakthrough advance in the science of bioenergetics:

$$pmf ( Δ p ) = Δ ψ + \frac{2.3 RT}{F} ( p {H}_{nB} + {log}_{10} ( \frac{c}{s} \cdot \frac{Δ ψ}{l \cdot F ( {\prod }_{i = 1}^{n} \{ {K}_{Pi} ( \frac{[ {M}_{pB}^{i +} ]}{[ {H}_{pB}^{+} ]} ) + 1 \} )} + [ {H}_{pB}^{+} ] ) ) \qquad [ 1 ]$$

Where Δψ is the electrical potential difference across the membrane; R is the gas constant; T is the absolute temperature in Kelvin (K); F is the Faraday constant; pH_{nB} is pH of the cytoplasmic (negative n side) bulk phase; [H⁺_{pB}] is the proton concentration in the periplasmic (positive p side) bulk aqueous phase such as in the case of alkalophilic bacteria; C/S is the specific membrane capacitance; l is the thickness for localized proton layer; K_{Pi} is the equilibrium constant for non-proton cations (Mⁱ⁺_{pB}) to exchange for localized protons; and [Mⁱ⁺_{pB}] is the concentration of non-proton cations in liquid culture medium (Lee 2015 Bioenergetics 4: 121. doi:10.4172/2167-7662.1000121).

The core concept of the proton-electrostatics localization hypothesis is based on the premise that a biologically-relevant water body, such as the water within a bacterium, can act as a proton conductor in a manner similar to an electric conductor with respect to electrostatics. This is consistent with the well-established knowledge that protons can quickly transfer among water molecules by the “hops and turns” mechanism. From the charge translocation point of view, it is noticed that hydroxyl anions are transferred in the opposite direction of proton conduction. This understanding suggests that excess free protons in a biologically-relevant water body behave like electrons in a perfect conductor. It is well known for a charged electrical conductor at static equilibrium that all extra electrons reside on the conducting body's surface. This is expected because electrons repel each other, and, being free to move, they will spread out to the surface. By the same token, it is reasonable to expect that free excess protons (or conversely the excess hydroxyl anions) in a biologically-relevant water body will move to its surface. Adapting this view to excess free hydroxyl anions in the cytoplasm (created by pumping protons across the cytoplasm membrane through the respiratory redox-driven electron-transport-coupled proton transfer into the liquid medium outside the cell), they will be electrostatically localized along the water-membrane interface at the cytoplasmic (n) side of the cell membrane such as in the case of alkalophilic bacteria. In addition, their negative charges (OH⁻) will attract the positively charged species (H⁺) outside the cell to the membrane-water interface at the periplasmic (p) side.

That is, when excess hydroxyl anions are created in the cytoplasm by the redox-driven proton pump across the membrane leaving excess protons outside the cell, the excess hydroxyl anions in the cytoplasm will not stay in the bulk water phase because of their mutual repulsion. Consequently, they go to the water-membrane interface at the cytoplasmic (n) side of the membrane where they then attract the excess protons at the periplasmic (p) side of the membrane, forming an “excess anions-membrane-excess protons” capacitor-like system. Therefore, the protonic capacitor concept is used to calculate the effective concentration of the ideal localized protons [H_{L}⁺]⁰ at the membrane-water interface in a pure water-membrane-water system assuming a reasonable thickness (l) for the localized proton layer using the following equation:

$${[ {H}_{L}^{+} ]}^{0} = \frac{C}{S} \cdot \frac{Δ ψ}{l \cdot F} = \frac{Δ ψ \cdot κ \cdot {ɛ}_{0}}{d \cdot l \cdot F} \qquad [ 2 a ]$$

where C/S is the membrane capacitance per unit surface area; F is the Faraday constant; κ is the dielectric constant of the membrane; ε₀ is the electric permittivity; d is the thickness of the membrane; and l is the thickness of the localized proton layer. This proton-capacitor equation [2a] is a foundation for the newly revised pmf equation [1], which includes an additional term that accounts for the effect of non-proton cations exchanging with the localized protons.

By rearranging Eq. 2a, we can also solve for the membrane potential ϕψ in terms of the ideal localized excess proton population density [H_{L}⁺]⁰ and the membrane capacitance properties including parameters such as the membrane capacitance per unit surface area C/S; the Faraday constant F; the membrane dielectric constant κ; the electric permittivity ε₀; the membrane thickness d; and the localized proton layer thickness l. Accordingly, the membrane potential Δψ can now be expressed as a function of the effective concentration of the ideal localized protons [H_{L}⁺]⁰ at the membrane-water interface in an idealized pure water-membrane-water system using the following equation:

$$Δψ = \frac{F \cdot S \cdot l \cdot {[ {H}_{L}^{+} ]}^{0}}{C} = \frac{F \cdot d \cdot l \cdot {[ {H}_{L}^{+} ]}^{0}}{κ \cdot {ɛ}_{o}} \qquad [ 2 b ]$$

From this equation [2b], it is now quite clear that it is the accumulation of excess protons and the resulting ideal localized proton density [H_{L}⁺]⁰ that essentially builds the membrane potential Δψ in proton-coupling bioenergetics systems.

Recently, using nanoscale measurements with electrostatic force microscopy, the dielectric constant (κ) of a lipid bilayer was determined to be about 3 units, which is in the expected range of 2˜4 units (Grames et al, Biophysical Journal 104: 1257-1262; Heimburg 2012 Biophysical Journal 103: 918-929.). Table 1 lists the calculation results for localized protons for an idealized pure water-membrane-water system with Eq. 2a using a lipid membrane dielectric constant κ of 3 units, membrane thickness d of 4 nm, trans-membrane potential difference Δψ of 180 mV, and three assumed values for the proton layer thickness of 0.5, 1.0, and 1.5 nm.

TABLE 1
Calculation of localized protons with Equation 2a in an idealized
pure water-membrane-water system using a membrane
dielectric constant κ of 3, membrane thickness d of 4 nm,
and trans-membrane potential difference Δψ of 180 mV.

| Assumed thickness (l) of ideal | 0.5 | nm | 1.0 | nm | 1.5 | nm |
|---|---|---|---|---|---|---|
| localized proton layer | | | | | | |
| Ideal localized proton density | 1.238 × 10⁻⁸ | | 1.238 × 10⁻⁸ | | 1.238 × 10⁻⁸ | |
| per unit area (moles H⁺/m²) | | | | | | |
| Effective concentration of ideal | 24.76 | mM | 12.38 | mM | 8.25 | mM |

| | | | |
|---|---|---|---|
| localized proton ([H_{L}⁺]⁰) | | | |
| Effective pH of ideal localized | 1.61 | 1.91 | 2.08 |
| proton layer (pH_{L}⁰) | | | |

As shown in Table 1, the ideal localized proton density per unit area was calculated to be 1.238×10⁻⁸ moles H⁺/m². The calculated effective concentration of ideal localized proton) ([H_{L}⁺]⁰) was in a range from 8.25 mM to 24.76 mM if the localized proton layer is around 1.0±0.5 nm thick. The calculated effective pH of localized proton layer (pH_{L}⁰) was 1.61, 1.91, and 2.08 assuming that the ideal localized proton layer is 0.5, 1.0, and 1.5-nm thick, respectively. This calculation result also indicated that localized excess protons may be created at a water-membrane interface for possible industrial applications such as acid-etching of certain metals and/or protonation of certain micro/nanometer materials without requiring the use of conventional acid chemicals such as nitric and sulfuric acids.

International Patent Application Publication No. WO2017/007762 A1 discloses a set of methods on creating electrostatically localized excess protons to be utilized as a clean “green chemistry” technology for industrial applications and, more importantly, as a special energy-renewing technology process to isothermally utilize environmental heat through electrostatically localized protons at a liquid-membrane interface for generation of local protonic motive force (equivalent to Gibbs free energy) to do useful work such as driving ATP synthesis. The discovery of this isothermal protonic environmental-heat-utilization energy-renewing process without being constrained by the Second Law of Thermodynamics may have seminal scientific and practical implications for energy and environmental sustainability on Earth. Further development and extension from this fundamental science and engineering breakthrough to the other fields such as the electron-based systems for energy renewal is highly desirable.

### SUMMARY OF THE INVENTION

The present invention revisits the systems of localized excess protons with new updates including protonic wires and, more importantly, discloses a series of methods on the creation and use of asymmetric function-gated isothermal electron power generator systems for isothermal electricity production by isothermally utilizing environmental heat energy which is also known as the latent (existing hidden) heat energy from the environment without requiring the use of conventional energy resources such as a high temperature gradient. A special energy-recycling and renewing technology is provided with the associated methods and systems to extract environmental heat energy including molecular and/or electron thermal motion energy for generating local protonic motive force (equivalent to Gibbs free energy) and more significantly for producing isothermal electricity to do useful work, which may have seminal scientific and practical implications for energy and environmental sustainability on Earth.

The present invention first describes a series of innovative methods that creates localized excess protons at a water-substrate or water-membrane interface that may be employed in combination of protonic wires for industrial process applications. According to one of the various embodiments, an open-circuit water electrolysis process uses a pair of anode and cathode electrodes in a special excess proton production and proton-utilization system, which can treat a series of substrate plate/film materials by forming and using an excess protons-substrate-hydroxyl anions capacitor-like system. The technology enables protonation and/or proton-driven oxidation of plate/film materials in a pure water environment in conjunction with a water-based protonic wire as a protonic scanner and/or writing tool. The present invention represents a remarkable clean “green chemistry” technology that does not require the use of any conventional acid chemicals including nitric and sulfuric acids for the said industrial applications and, more importantly, as a special tool to utilize latent heat energy from the environment for generation of local proton motive force (equivalent to Gibbs free energy) to do useful work such as driving ATP synthesis.

Creating and using excess protons-substrate-hydroxyl anions capacitor-like systems has been demonstrated through an experimental study. According to this experimental study, excess protons do not stay in a bulk water liquid phase in the anode chamber. Instead, they electrostatically localize at the water-membrane interface at the anode chamber and attract the excess hydroxyl anions of the cathode chamber water to the other side of the substrate film. The effective concentration of localized protons at the water-membrane interface can be well above 0.1 mM, making them potent enough to enable protonation of synthetic substrate materials such as (poly) aniline. The use of localized excess protons as a micro/nanometer tool can also perform proton-etching of certain substrate materials such as aluminum, iron, and copper to create various desirable proton-etching patterns on a substrate membrane, film, or a substrate plate.

Since the excess-proton treatment such as the protonation of synthetic substrate materials such as (poly)aniline or proton-etching of micro/nanometer materials can be operated in a pure water environment with a neutral bulk-phase pH, when the so-treated substrate is taken out of the pure water chamber system, it could immediately emerge as a clean quality product (any residual pure water can be readily dried off) without requiring any additional washing/cleaning step that a conventional acid-treatment process would require. Therefore, the method disclosed in this invention represents a remarkably clean “green chemistry” technology.

The application of localized excess protons with a liquid membrane chamber system provides a special energy-recycling and renewing technology process function to extract environmental heat from ambient temperature environment including the molecular thermal motion energy for generating local protonic motive force (equivalent to Gibbs free energy) to do useful work such as driving ATP synthesis.

According to one of the various embodiments, the liquid membrane chamber system is a multi-chamber excess proton production and utilization system comprising: a) Multiple membranes are placed in between an anode chamber and a cathode chamber, forming multiple induction chambers among multiple membranes; b) Chamber wall is made of water- and proton-impermeable, chemically-inert and electrically insulating materials; c) Proton users comprising ATP synthase are embedded with each of the multiple membranes.

According to another of the various embodiments, the special energy-recycling and renewing technology process has a special feature that employs multiple membranes, each with a relatively smaller membrane potential, in a multi-chamber system that can be employed with use of a relatively small electrolysis voltage for generating excess protons to extract environmental heat molecular thermal motion energy to create a total pmf value much larger than the input electrolysis voltage.

According to another of the various embodiments, the special energy-recycling and renewing technology process to extract environmental heat energy associated with localized protons for generating local protonic motive force (equivalent to Gibbs free energy) comprising the following steps and features: a) Through use of the “open-circuit” water-electrolysis process, excess protons are generated in anode liquid chamber while excess hydroxyl anions are created in cathode liquid chamber; b) The generated excess protons electrostatically localize themselves primarily along the water-membrane interface at the positive (anodic) interface (PI) site while the excess anions electrostatically localize themselves primarily along the water-membrane interface at the negative (cathodic) interface (NI) site; c) The excess protons at PI site in conjunction with the excess anions at NI site electrostatically induce the formation of induced anions at the induced negative interface (INI) site(s) and the induced protons at the induced positive interface (IPI) site(s) in the induction liquid chambers; d) The formation of the electrostatically localized protons at the water-membrane interface constitutes a type of “negative entropy” event resulting in the formation of multiple “localized protons-membrane-anions” capacitor-like structures; e) The formation of multiple “localized protons-membrane-anions” capacitor-like structures results in the formation of membrane potential across each of the membranes; f) In addition to the generation of membrane potential, significant amount of “bonus” local proton motive force is also created from the “entropy effect” of the localized protons since their thermal molecular motion energy can drive nanometer scale molecular machines such as F₀F₁-ATP synthase embedded in the membrane.

According to another of the various embodiments, the special energy-recycling and renewing technology process has a preferred practice to place the proton-generating anode electrode well into the bulk phase liquid and to keep the mouths of proton users being located rightly within the localized excess protons layer along the membrane surface, for the best effect to utilize the environmental heat associated with the molecular thermal motion energy of localized protons to perform useful work such as driving the synthesis of ATP, enhancing the protonation of certain synthetic polymer materials, and driving the proton-etching of certain substrate metal plates.

According to exemplary embodiments, the utilization of environmental heat with localized protons to recycle/utilize the fully dissipated waste heat energy, which conventionally is thought to be totally unusable, generates local pmf to do useful work. This provides an innovative method to renew the totally “dead” heat energy in ambient temperature environment that according to the Second Law of Thermodynamics would be completely unusable. That is, the “dead” latent heat energy can now be reborn to create new Gibbs free energy in the form of local pmf in accordance with the present invention. Therefore, it fundamentally represents a special energy-recycle and energy-renew-related technology.

According to one of the exemplary embodiments, a water-based protonic wire comprises a proton-conductive water line filled in a protonic insulating tube and/or a channel, which may be used in making protonic circuits. The water-based protonic wires and protonic circuits may be employed in combination of localized excess protons for certain industrial processes and/or for certain biomedical science and technology applications. For example, the micro/nanometer-scale water-based protonic wires and artificial protonic circuits may be used to interact with certain human and/or animal tissue cells such as neurons for certain biomedical diagnosis and/or surgery treatments.

The present invention further discloses an energy renewal method for generating isothermal electricity with making and using a special asymmetric function-gated isothermal electricity power generator system comprising at least one pair of a low work function thermal electron emitter and a high work function electron collector across a barrier space installed in a container (such as a vacuum tube, bottle or chamber) with electric conductor support to enable a series of energy recycle process functions with isothermal utilization of environmental heat energy for at least one of: a) utilization of environmental heat energy for energy recycling and renewing of fully dissipated waste heat energy from the environment to generate electricity with an output voltage and electric current to do useful work; b) providing a novel cooling function for a new type of freezer/refrigerator without requiring any of the conventional refrigeration mechanisms of compressor, condenser, evaporator and/or radiator by isothermally extracting environmental heat energy from inside the freezer/refrigerator while generating isothermal electricity; and c) combinations thereof.

According to one of the exemplary embodiments, the present invention teaches the making and using of an asymmetric function-gated isothermal electron-based power generator system that has a low work function (0.7 eV) Ag—O—Cs emitter and a high work function Cu metal (4.56 eV) collector installed in a chamber-like vacuum tube comprising: a Ag—O—Cs film coated on the dome-shaped top end inner surface of the chamber-like vacuum tube to serve as the emitter; a vacuum space allowing thermally emitted electrons to fly through ballistically between the emitter and collector; a Cu film coated on the inversed-dome-shaped bottom end inner surface of the chamber-like vacuum tube to serve as the collector; a first electricity outlet (such as an electric conductive wire and/or lead) connected with the emitter; and a second electricity outlet connected with the collector.

According to one of the exemplary embodiments, the present invention teaches the making and using of an integrated isothermal electricity generator system that has a narrow inter electrode space gap size for each of three pairs of emitters and collectors installed in a vacuum tube chamber set up vertically comprising: a low work function film coated on the first electric conductor plate bottom surface to serve as the first emitter; a first narrow space allowing thermally emitted electrons to flow through ballistically between the first pair of emitter and collector; a high work function film coated on the second electric conductor top surface to serve as the first collector; a low work function film coated on the second electric conductor bottom surface to serve as the second emitter; a second narrow space allowing thermally emitted electrons to flow through ballistically between the second pair of emitter and collector; a high work function film coated on the third electric conductor top surface to sever as a collector; a low work function film coated on the third electric conductor bottom surface to serve as the third emitter; a third narrow space allowing thermally emitted electrons to flow through ballistically between the third pair of emitter and collector; a high work function film coated on the fourth electric conductor top surface to serve as the terminal collector, a first electricity outlet (wire) and an Earth ground that are connected with the first electric conductor plate; and a second electric outlet (wire) that is connected with the fourth electric conductor.

According to one of the exemplary embodiments, the effect of an asymmetric function-gated isothermal electricity production is additive. Pluralities (n) of asymmetrically function-gated isothermal electricity generator systems may be employed in parallel and/or in series. When a plurality (n) of the asymmetric function-gated isothermal electricity generator systems are used in parallel, the total steady-state electrical current (Iₛₜ₍ₜₒₜₐₗ₎) is the summation of the steady-state electrical current (Iₛₜ₍ᵢ₎) from each of the asymmetrically function-gated isothermal electricity generator systems while the total steady-state output voltage (Vₛₜ₍ₜₒₜₐₗ₎) remains the same. Conversely, when a plurality (n) of the asymmetric function-gated isothermal electricity generator systems operate in series, the total steady-state output voltage (Vₛₜ₍ₜₒₜₐₗ₎) is the summation of the steady-state output voltages (Vₛₜ₍ᵢ₎) from each of the asymmetrically function-gated isothermal electricity generator systems while the total steady-state electrical current (Iₛₜ₍ₜₒₜₐₗ₎) remains the same.

According to one of the exemplary embodiments, the present invention teaches the making and using of an integrated isothermal electricity generator system that employs three pairs of exceptionally low work function Ag—O—Cs (0.5 eV) emitters and high work function Au metal (5.10 eV) collectors working in series comprising: a Ag—O—Cs film coated on the dome-shaped top end inner surface of the vacuum tube chamber to serve as the first emitter that has an electricity outlet; a first vacuum space allowing thermally emitted electrons to flow through ballistically across the first pair of emitter and collector; a Au film coated on the first middle electric conductor top surface to serve as the first collector; a Ag—O—Cs film coated on the first middle electric conductor bottom surface to serve as the second emitter; a second vacuum space allowing thermally emitted electrons to flow through ballistically across the second pair of emitter and collector; a Au film coated on the second middle electric conductor top surface to serve as the second collector; a Ag—O—Cs film coated on the second middle electric conductor bottom surface as the third emitter; a third vacuum space allowing thermally emitted electrons to flow through ballistically across the third pair of emitter and collector; and an Au film coated on the inversed-dome-shaped bottom end inner surface of the vacuum tube chamber to serve as the terminal collector connected with an electricity outlet.

According to another one of the exemplary embodiments, the present invention teaches the making and using of an asymmetric function-gated isothermal electricity generator system that has a pair of an exceptionally low work function Ag—O—Cs (0.5 eV) emitter and a high work function graphene (4.60 eV) collector is employed to provide cooling for a new type of novel freezer/refrigerator by isothermally extracting environmental heat energy from inside the freezer/refrigerator while generating isothermal electricity.

### BRIEF DESCRIPTION OF THE DRAWINGS

FIG. 1a presents an embodiment that produces excess protons and hydroxyl anions through an “open-circuit” water electrolysis process and results in an “excess protons-substrate-hydroxyl anions” capacitor-like system for industrial applications.

FIG. 1b presents an embodiment showing that the excess proton monolayer at the water-membrane interface is extended from a secondary proton layer of the “electric double layer” that covers the anode surface when electrolysis voltage is applied.

FIG. 1c presents an embodiment showing the likely distribution of excess protons and excess hydroxyl anions in the two water chambers separated by a membrane when electrolysis voltage is turned off.

FIG. 2 presents a three-chamber system that produces excess protons and hydroxyl anions through an “open-circuit” water electrolysis process and results in two “excess protons-substrate-hydroxyl anions” capacitor-like structures for industrial applications.

FIG. 3 presents a four-chamber system that produces excess protons and hydroxyl anions through an “open-circuit” water electrolysis process and results in three “excess protons-substrate-hydroxyl anions” capacitor-like structures for industrial applications.

FIG. 4 presents a multi-chamber system that produces excess protons and hydroxyl anions forming multiple “excess protons-membrane-hydroxyl anions” capacitor-like structures for extraction of environmental heat molecular motion energy to generate additional protonic motive force (equivalent to Gibbs free energy) to do useful work such as driving ATP synthesis.

FIG. 5 presents: (a) A top view photograph showing the ElectroPrep apparatus. Pieces of proton-sensitive aluminum (Al) films were applied on the water surface and in the middle (bulk phase) of both the anode and cathode water chambers. Nylon strings were used to anchor the pieces of proton-sensitive films that were suspended in the middle of both the anode and cathode water chambers. (b) polytetrafluoroethylene, e.g., Teflon (Tf), center chamber assembly with a Tf-Al-Tf membrane. (c) polytetrafluoroethylene, e.g., Teflon, center chamber assembly with a proton-sensing Al-Tf-Al membrane.

FIG. 6 presents the observations with proton-sensing Al films after 10 hours of cathode water Al-Tf-Al water anode experiment with water electrolysis (200 V). N_{I}: Proton-sensing film at the N side of Teflon membrane detected no proton activity. P_{I}: Proton-sensing film at the P side of Teflon membrane detected dramatic activity of localized protons (dark grey color). N_{B}: Proton-sensing film suspended inside the water of the cathode chamber. N_{S}: Proton-sensing film floating on the water surface of cathode chamber. P_{S}: Proton-sensing film floating on the water surface of anode chamber. P_{B}: Proton-sensing film suspended inside the bulk water phase of the anode chamber.

FIG. 7 presents the electric current of water electrolysis measured as a function of time with 200 V during 10 hours experimental run. The electric current curve marked with “Al-Tf-Al expt” shows average of three experiments with the “cathode water Al-Tf-Al water anode” system. The electric current curve marked with “Tf-Al-Tf expt” shows average of three experiments with the “cathode water Tf-Al-Tf water anode” set up; and its initial part within the first 2000 seconds is plotted in an expanded scale showing the integration for the area under the curve (Inset).

FIG. 8a presents a photograph showing the Teflon center chamber with the Al-Tf-Al membrane (at the P_{I} site) seen through the anode chamber water after the 10-hour experiment with generation of excess protons through water electrolysis. Formation of gas bubbles and significant localized proton activity was noticed on the aluminum membrane surface at the P_{I} site.

FIG. 8b presents a photograph showing the ElectroPrep electrolysis system after 10-hour water electrolysis. Notice, the proton-sensing Al film held in the bulk water phase (P_{B}) near the middle of the anode chamber (right) showed no excess proton activity as it still retaining pristine during the entire 10-hour experiment. The bulk water phase pH was measured by inserting the probe into the anode (right side) and cathode (left side) water chambers.

FIG. 9 presents the experimental evidence in detection of protons with proton-sensing films at the P′, P, N and N′ sites in the cathode water-Teflon chamber water-anode water system.

FIG. 10 presents a photograph of the cathode water-Teflon chamber water-anode water system with a piece of proton-sensing Al film material inserted into the anode chamber water body (at the right side).

FIG. 11 presents the experimental evidence in detection of electrostatically localized protons with proton-sensing Al films at the P and P′ sides in a cathode water-Teflon induction chamber sodium bicarbonate solution-anode water system with the presence of 10 mM (top row), 100 mM (bottom row), and 400 mM (middle bottom row) of sodium bicarbonate water solution in the center Teflon induction chamber.

FIG. 12 presents the calculated total, local and classic pmf values of Bacillus pseuodofirmus OF4 as a function of the external (liquid culture medium) pH compared to the minimum value (116 mV) required to synthesize ATP and to the maximum value (redox potential energy limit: 228 mV) that could be supported thermodynamically by the redox-driven proton pump system as allowed by the Second Law of Thermodynamics.

FIG. 13 presents an asymmetric function-gated isothermal electron power generator system 1000 comprising an asymmetric electron-gating function across a membrane-like barrier space that separates two electric conductors.

FIG. 14a presents a basic unit of an asymmetric function-gated isothermal electron power generator system 1100 comprising a barrier space such as a vacuum space that separates a pair of electric conductors: one of them has a low work function film to act as a thermal electron emitter and the other has a high work function plate surface to serve as an electron collector.

FIG. 14b illustrates certain characteristics in the asymmetric function-gated isothermal electricity generator system 1100 such as the excess holes (positive charges) left at the emitter will also electrostatically spread to the surface, and likewise so do the excess electrons at the collector under the “open circuit” condition.

FIG. 14c illustrates a preferred practice to ground the emitter with an Earth ground at the electricity outlet 1106 terminal of the asymmetric function-gated isothermal electricity generator system 1100.

FIG. 15 presents the energy diagrams of the asymmetric function-gated isothermal electron power generator system 1100.

FIG. 16a presents an example for a pair of silver (Ag) and molybdenum (Mo) electrodes installed in a vacuum tube as part of a fabrication process to create an asymmetric function-gated isothermal electricity generator system.

FIG. 16b presents an example of a prototype isothermal electricity generating system using a low work function Ag—O—Cs film coated on the silver electrode surface to serve as a thermal electron emitter.

FIG. 17a presents examples of the isothermal electricity current density (A/cm²) as a function of operating temperature T at various output voltage V(c) from 0.00 to 3.86 V, as calculated using Eq. 12 for a pair of low work function (0.70 eV) emitter and high work function (4.56 eV) collector; in which the emitter was grounded.

FIG. 17b presents examples of the isothermal electricity current density curves as a function of output voltage V(c) from 0.00 to 3.86 V at an operating temperature of 273, 293, 298, or 303 K for a pair of low work function (0.70 eV) emitter and high work function (4.56 eV) collector; in which the emitter was grounded.

FIG. 17c presents examples of the isothermal electricity current density (A/cm²) curves at an output voltage V(c) of 3.00 V as a function of operating environmental temperature T for a series of emitters with a low work function of 0.4, 0.5, 0.6, 0.7, 0.8, 0.9, 1.0, 1.1, or 1.2 eV; each of these emitters is grounded and paired with a high work function (4.56 eV) collector.

FIG. 18a presents examples of the isothermal electricity current density (A/cm²) curves as a function of output voltage V(c) from 0.00 to 5.31 V at an operating environmental temperature of 273, 293, 298, and 303 K for a pair of low work function (0.6 eV) emitter and high work function (5.91 eV) collector; in which the emitter was grounded.

FIG. 18b presents examples of the isothermal electricity current density (A/cm²) as a function of operating environmental temperature T for a series of emitters with low work function values including 0.4, 0.5, 0.6, 0.7, 0.8, 0.9, 1.0, 1.1, 1.2, 1.3, 1.4, 1.5, 1.6, 1.8, 2.0, or 2.2 eV; each of these emitters is grounded and paired with a high work function (5.91 eV) collector.

FIG. 18c presents examples of the isothermal electricity current density (A/cm²) at an output voltage V(c) of 4.00 V as a function of operating environmental temperature T for a series of emitters with low work function values including 0.4, 0.5, 0.6, 0.7, 0.8, 0.9, 1.0, 1.1, 1.2, 1.3, 1.4, 1.5, 1.6, 1.8, or 2.0 eV; each of these emitters is grounded and paired with a high work function (5.91 eV) collector.

FIG. 18d presents examples of the isothermal electricity current density (A/cm²) at an output voltage V(c) of 5.00 V as a function of operating environmental temperature T for a series of emitters with low work function values including 0.4, 0.5, 0.6, 0.7, 0.8, or 0.9 eV; each of these emitters is grounded and paired with a high work function (5.91 eV) collector.

FIG. 19a presents examples of the isothermal electricity current density (A/cm²) curves as a function of output voltage V(c) volts from 0.00 to 4.10 V at an operating environmental temperature of 273, 293, 298, or 303 K for a pair of emitter work function (0.50 eV) and collector work function (4.60 eV), with the emitter grounded.

FIG. 19b presents examples of the isothermal electricity current density (A/cm²) curves as a function of output voltage V(c) volts from 0.00 to 4.10 V at freezing/refrigerating temperature of 253, 263, 273, or 277 K for a pair of emitter work function (0.50 eV) and collector work function (4.60 eV), with the emitter grounded.

FIG. 19c presents examples of the isothermal electricity current density (A/cm²) as a function of operating environmental temperature T for a series of emitters with low work function values including 0.4, 0.5, 0.6, 0.7, 0.8, 0.9, 1.0, 1.1, 1.2, 1.3, 1.4, 1.5, 1.6, 1.8, 2.0, 2.2, 2.4, 2.6, 2.8, 3.0, or 3.5 eV; each of these emitters is grounded and paired with a high work function (4.60 eV) collector.

FIG. 20 presents an example of an integrated isothermal electricity generator system 1300 that comprises multiple (e.g., three) pairs of emitters and collectors working in series.

FIG. 21a presents an example of a prototype for an isothermal electricity generator system 1400A that has a pair of emitter (work function 0.7 eV) and collector (work function 4.36 eV) installed in a container such as a vacuum tube chamber.

FIG. 21b presents an example of a prototype for an isothermal electricity generator system 1400B that has two pairs of emitters (work function 0.7 eV) and collectors (work function 4.36 eV) installed in a vacuum tube chamber.

FIG. 21c presents an example of a prototype for an integrated isothermal electricity generator system 1400C that comprises three pairs of emitters (work function 0.7 eV) and collectors (work function 4.36 eV) installed in a vacuum tube chamber.

FIG. 22 presents an example of an integrated isothermal electricity generator system 1500 that has a narrow inter electrode space gap size for each of three pairs of low work function emitters and high work function collectors installed in a vacuum tube chamber set up vertically.

FIG. 23 presents an example of an integrated isothermal electricity generator system 1600 that has three pairs of low work function emitters and high work function collectors installed in a vacuum tube chamber set up vertically to utilize the gravity to help pull the emitted electrons from an emitter down to a collector.

FIG. 24a presents an example of an isothermal electricity generator system 1700A that has a pair of low work function Ag—O—Cs (0.6 eV) emitter and high work function protonated polyaniline (4.42 eV) collector installed in a chamber-like vacuum tube container.

FIG. 24b presents an example of an integrated isothermal electricity generator system 1700B that has two pairs of low work function Ag—O—Cs (0.6 eV) emitters and high work function of protonated polyaniline (4.42 eV) collectors working in series as installed in a chamber-like vacuum tube container.

FIG. 24c presents an example of an integrated isothermal electricity generator system 1700C that has three pairs of low work function Ag—O—Cs (0.6 eV) emitters and high work function protonated polyaniline (4.42 eV) collectors operating in series as installed in a vacuum tube container.

FIG. 25a presents another example of an isothermal electricity generator system 1800A that has a pair of low work function Ag—O—Cs (0.7 eV) emitter and high work function Cu metal (4.56 eV) collector installed in a chamber-like vacuum tube container.

FIG. 25b presents another example of an integrated isothermal electricity generator system 1800B that has two pairs of low work function Ag—O—Cs (0.7 eV) emitters and high work function of Cu metal (4.56 eV) collectors operating in series as installed in a chamber-like vacuum tube container.

FIG. 25c presents another example of an integrated isothermal electricity generator system 1800C that has three pairs of low work function Ag—O—Cs (0.7 eV) emitters and high work function Cu metal (4.56 eV) collectors operating in series as installed in a vacuum tube container.

FIG. 26 presents an example of an integrated isothermal electricity generator system 1900 that employs three pairs of exceptionally low work function Ag—O—Cs (0.5 eV) emitters and high work function Au metal (5.10 eV) collectors operating in series as installed in a vacuum tube container.

FIG. 27 presents an example of an integrated isothermal electricity generator system 2000 that employs three pairs of low work function doped-graphene (1.01 eV) emitters and high work function graphite (4.60 eV) collectors operating in series as installed in a vacuum tube container.

FIG. 28 presents an example of an integrated isothermal electricity generator system 2100 that has three pairs of low work function doped-graphene (1.01 eV) emitters and high work function graphene (4.60 eV) collector operating in series as installed in a vacuum tube container.

FIG. 29a presents photographs for a pair of parallel aluminum plate-supported silver (Ag) and copper (Cu) electrode plates (size: 40 mm×46 mm) held together with electric-insulating plastic spacers (washers), screws and nuts at the four corners for each of the two electrode plates to make a pair of Ag—O—Cs type emitter (CsOAg) and Cu collector with or without oxygen plasma treatment.

FIG. 29b presents photographs for a pair of parallel aluminum plate-supported silver (Ag) and copper (Cu) collector electrode plates (size: 40 mm×46 mm) held together with electric-insulating plastic spacers (washers), heat-shrink plastic tube-insulated metal screws and nuts at the corners of the electrode plates. The silver (Ag) plate and copper (Cu) collector plate were connected by soldering with a red insulator coated copper wire and a blue insulator coated copper wire, respectively. The silver (Ag) electrode plate surface was coated with a thin molecular layer of cesium oxide (Cs₂O) through painting with a dilute cesium oxide solution followed by drying to form a type of Ag—O—Cs emitter (CsOAg) with or without oxygen plasma treatment.

FIG. 30 presents a photograph of the parts for a prototype CsOAg—Cu electrobottle that comprise a pair of parallel aluminum plate-supported CsOAg (silver (Ag), coated with Cs₂O) and copper (Cu) collector plates installed with the red and blue insulator coated copper wires passing through a screw bottle cap. Two blue plastic air tubes were installed through two additional holes in the screw bottle cap. Electric-insulating and air-tight Kafuter 704 RTV silicone gel (white) was used to seal the joints for the wires and tubes passing through the bottle cap.

FIG. 31a presents a photograph showing four prototype CsOAg—Cu electrobottles that were fabricated using crew bottle caps. Each electrobottle comprises a pair of parallel aluminum plate-supported silver CsOAg (a type of Ag—O—Cs emitter) and copper (Cu) collector electrode surfaces installed with red and blue insulator coated wires passing through a screw bottle cap. After installation and sealing with electric-insulating and air-tight Kafuter 704 RTV silicone gel (white), air was removed from each of the electro-bottles using a vacuum pump through the blue plastic tubes with the bottle cap.

FIG. 31b presents a photograph of 17 prototype CsOAg—Cu electro-bottles that were made using non-screw bottle caps and sealed with electric-insulating and air-tight Kafuter 704 RTV silicone gel (white) material.

FIG. 32a presents a photograph showing a prototype CsOAg—Cu electrobottle that was placed into a Faraday box for isothermal electricity production testing by connecting its red and blue insulator coated copper wires (passing the non-screw bottle cap) with Keithley 6514 electrometer system's Model 237-ALG-2 low noise cable-alligator clips.

FIG. 32b presents a photograph of a Faraday box made of heavy-duty aluminum foils containing a prototype CsOAg—Cu electrobottle inside for isothermal electricity production testing with a Keithley 6514 system electrometer.

FIG. 33a presents a photograph of a prototype CsOAg—Cu electrobottle placed inside a Faraday box and tested in normal polarity (Keithley 6514 red alligator connector to CsOAg emitter plate and black alligator connector to Cu collector plate), showing an electric current reading of “11.888 pA·CZ”.

FIG. 33b presents a photograph of a prototype CsOAg—Cu electrobottle placed inside a Faraday box and tested in reverse polarity (Keithley 6514 black alligator connector to CsOAg emitter plate and red alligator connector to Cu collector plate), showing an electric current reading of “−11.030 pA·CZ”.

FIG. 34a presents a photograph of a prototype CsOAg—Cu electrobottle placed inside a Faraday box and tested in normal polarity (Keithley 6514 red alligator connector to CsOAg emitter plate and black alligator connector to Cu collector plate), showing an electric voltage reading of “0.10051 V·CZ”.

FIG. 34b presents a photograph of a prototype CsOAg—Cu electrobottle placed inside a Faraday box and tested with an electric shorting wire between the terminals (outlets) of CsOAg emitter and Cu collector, showing an electric voltage reading of “−0.00001 V·CZ”.

FIG. 34c presents a photograph of a prototype CsOAg—Cu electrobottle placed inside a Faraday box and tested in reverse polarity (Keithley 6514 black alligator connector to CsOAg emitter and red alligator connector to Cu collector, showing an electric voltage reading of “−0.11329 V·CZ”.

FIG. 35 presents a photograph of two prototype CsOAg—Cu electrobottles connected in parallel in normal polarity (Keithley 6514 red alligator connector to CsOAg emitter plates and black alligator connector to Cu collector plates) inside a Faraday box, showing an electric current reading of “22.230 pA·CZ”.

FIG. 36 presents a photograph of three prototype CsOAg—Cu electrobottles connected in parallel with their normal polarity (Keithley 6514 red alligator connector to CsOAg emitter plates and black alligator connector to Cu collector plates) inside a Faraday box, showing an electric current reading of “26.166 pA·CZ”.

### DETAILED DESCRIPTION

The present invention revisits the systems of localized excess protons and discloses a series of methods on the creation of asymmetric function-gated isothermal electron power generator systems for isothermal electricity production by isothermally utilizing latent (existing hidden) heat energy from the environment without requiring the use of conventional energy resources such as a high temperature gradient.

Accordingly, a special energy-recycling and renewing technology is disclosed with the associated methods to extract environmental heat energy including molecular and/or electron thermal motion energy for generating local proton motive force (equivalent to Gibbs free energy) and more significantly for producing isothermal electricity to do useful work, which may have seminal scientific and practical implications for energy and environmental sustainability on Earth. In particular, the present invention discloses an energy renewal method for generating isothermal electricity with a special asymmetric function-gated isothermal electricity power generator system comprising at least one pair of a low work function thermal electron emitter and a high work function electron collector across a barrier space installed in a container such as a bottle with electric conductor support to enable a series of energy recycle process functions with utilization of environmental heat energy isothermally for at least one of: a) utilization of environmental heat energy for energy recycling and renewing of fully dissipated waste heat energy from the environment to generate electricity with an output voltage and electric current to do useful work; b) providing a novel cooling function for a new type of freezer/refrigerator without requiring any of the conventional refrigeration mechanisms of compressor, condenser, evaporator and/or radiator by isothermally extracting latent energy from inside the freezer/refrigerator while generating isothermal electricity; and c) combinations thereof. The various aspects of the present invention are described in further details starting from the proton-based systems and then to the asymmetric function-gated isothermal electron power generator systems hereinbelow.

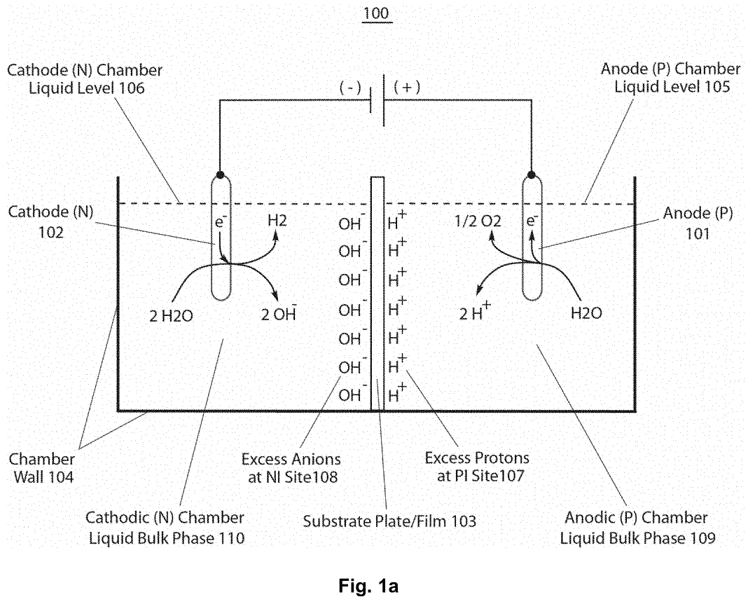

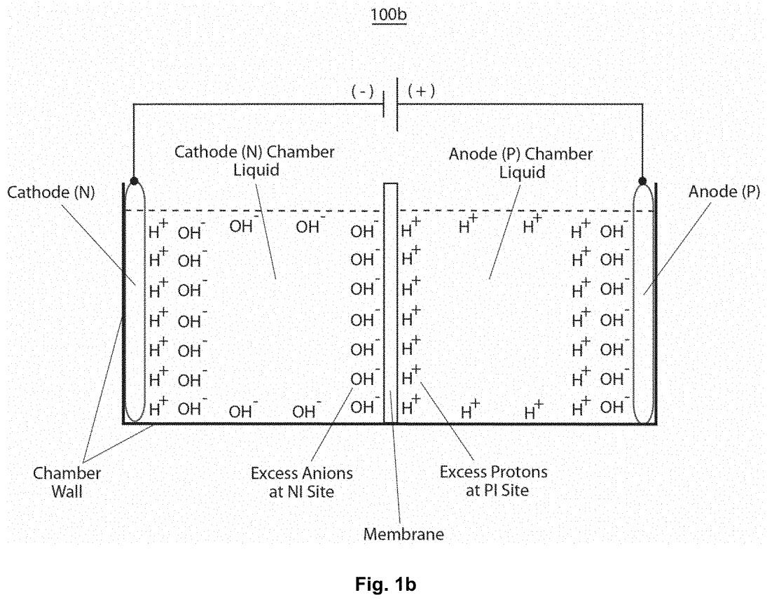

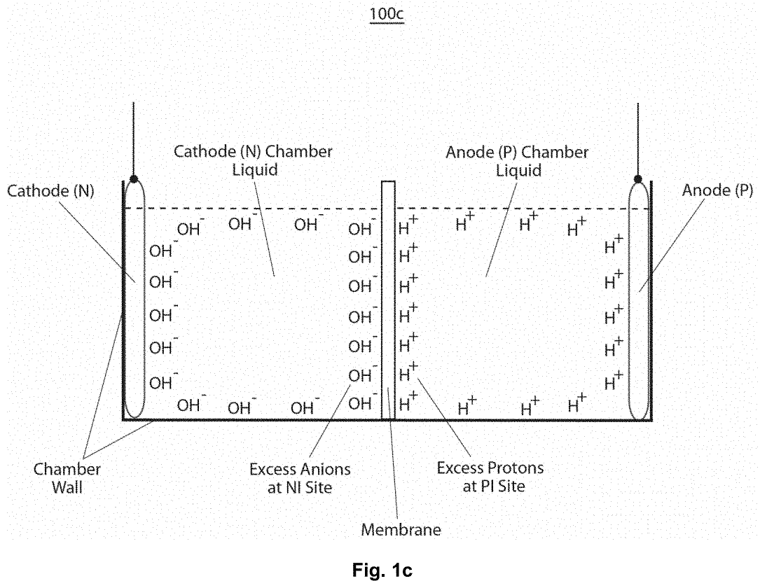

Referring to FIG. 1a, in one embodiment, an excess proton production system 100 is illustrated. The excess proton production system includes a substrate plate/film 103 that is placed in between an anode chamber and a cathode chamber. The chamber wall 104 is made of water- and proton-impermeable and chemically-inert materials such as Teflon, plastic material, and glass, which are unreactive even if under high power voltage. The substrate plate/film (membrane) 103 joins with the chamber wall 104 using a water-tight seal, resulting in the two separate chambers: the anode water chamber and the cathode water chamber.

According to one of the various embodiments, both the anode and cathode chambers are filled with pure water. The anode (N) chamber liquid level 105 is set preferably at the same level as the cathode (N) chamber liquid level 106. Both the anode (P) 101 and cathode (N) 102 are typically made of stable electrode materials such as metallic platinum, palladium, gold, copper, certain stainless steels, graphite, micro/nanometer carbon fiber materials and combinations thereof. The anode and cathode are placed into the anodic (P) liquid bulk phase 109 and the cathodic (N) liquid bulk phase 110, respectively. The excess protons and excess hydroxyl anions are generated through the use of “open-circuit” water-electrolysis by applying a direct current (DC) voltage across the anode (P) 101 and cathode (N) 102 electrodes (FIG. 1a) in the two water bodies separated by the substrate plate/film 103. In accordance with one of the various embodiments, the excess protons produced by the “open-circuit” water-electrolysis process localize at the water-substrate interface (PI Site 107) along the surface of the plate/film (or membrane) where they attract the excess hydroxyl anions at the other side (NI Site 108) of the substrate plate, forming an “excess protons-plate-excess anions” capacitor-like structure.

According to one of the various embodiments, the direct current (DC) electric voltage applied across the anode and cathode electrodes is selected from the group consisting of 1.23 V, 1.5 V, 2 V, 3 V, 4 V, 5 V, 6 V, 7 V, 8 V, 10 V, 11 V, 12 V, 13 V, 14 V, 15 V, 17 V, 18 V, 19 V, 20 V, 21 V, 22 V, 23 V, 24 V, 25 V, 26 V, 27 V, 28 V, 29 V, 30 V, 31 V, 32 V, 35 V, 36 V, 40 V, 50 V, 60 V, 70 V, 80 V, 90 V, 100 V, 150 V, 200 V, 250V, 300 V, 400 V, 500 V, 600 V, 700 V, 800 V, 900 V, 1000V, 1200 V, 1500 V, 2000 V, 2500 V, 3000 V, 4000V, 5000 V, 6000 V, 8000 V, 10,000 V, 12,000 V, 15,000 V, 20,000 V, 25,000V, 30,000 V and/or within a range bounded by any two of these values. When necessary to work with a voltage above 36 V, certain electric safety protocol must be strictly followed to prevent any electric shocks and accidents.

The effective concentration of the localized excess protons at the water-substrate interface can be at a value selected from the group consisting of 0.1 mM, 1 mM, 2 mM, 3 mM, 5 mM, 10 mM, 20 mM, 30 mM, 40 mM, 50 mM, 60 mM, 70 mM, 80 mM, 90 mM 100 mM, 120 mM, 150 mM, 200 mM, 300 mM, 500 mM, 1 M, 2 M, 3 M, 5M, 10 M and/or within a range bounded by any two of these values, which are often orders of magnitude higher than that indicated by the bulk water liquid phase pH measurements. Therefore, according to one of the various embodiments, this type of localized excess protons may be utilized to protonate certain special materials such as (poly)aniline and/or to treat certain synthetic materials and metal surfaces such as aluminum, iron and copper by “acid etching” or oxidation by protons.

According to one of the various embodiments, the substrate plate/film (or membrane) is selected from the group consisting of protonatable materials such as (poly)aniline, and certain metal surfaces such as aluminum, iron, copper, and combinations thereof.

According to one of the various embodiments, the protonatable materials such as (poly)aniline can be protonated at the PI site 107 (FIG. 1a) as shown in the following protonation reaction:

(—R—NH—)ₙ+n H⁺=(—R—NH₂⁺—)ₙ [3]

Whereas certain polymer substrate such as a protonated (poly)aniline film may be deprotonated at the NI site 108 (FIG. 1a) according to the following de-protonation process reaction:

(—R—NH₂⁺—)ₙ+n HO⁻=(—R—NH—)ₙ+n H₂O [4]

Therefore, use of this invention can create a special polymer film with an asymmetric proton distribution across the film material that may confer certain special functions such as diodic properties.

According to one of the various embodiments, certain metal surfaces such as aluminum, iron, and copper can be etched or oxidized by the excess protons at the PI site 107 (FIG. 1a) through the following proton-etching process reaction:

Al+2H⁺=Al⁺⁺+H₂ [5]

This proton-etching process differs from the conventional metal electroetching process that involves the use of a solution of an electrolyte (salt) rather than pure water. In the conventional metal electroetching process, the metal piece to be etched is connected to the positive pole of a source of direct electric current. A piece of the same metal is connected to the negative pole of the direct current source and is called the cathode. In order to reduce unwanted electro-chemical effects, the anode and the cathode conventionally should be of the same metal. Similarly, the cation of the electrolyte should be of the same metal as well. When the current source is turned on, the metal of the anode is dissolved and converted into the same cation as in the electrolyte and at the same time an equal amount of the cation in the solution is converted into metal and deposited on the cathode.

In contrast, the proton-etching process does not require any electrolyte (salt) since it uses pure water. Furthermore, the metal piece (substrate 103) to be etched is not directly connected with the anode. Consequently, the metal of the anode 101 is not dissolved and there is no metal deposition at the cathode 102.

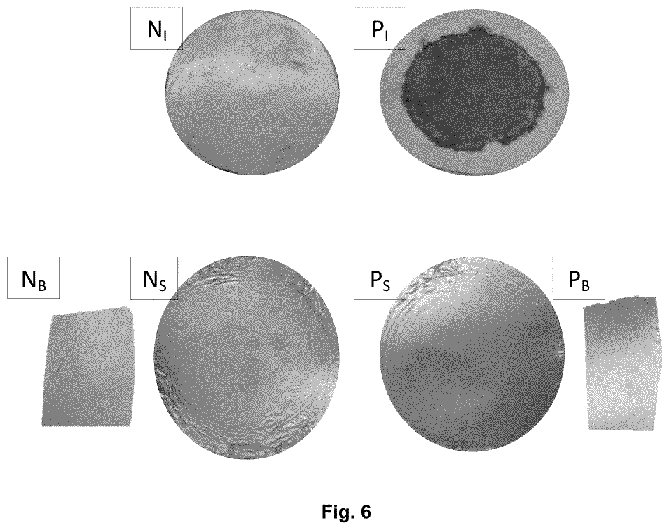

According to one of the various embodiments, this proton-etching process may be employed as a micro/nanometer fabrication tool with an acid-resistant material “resist” as mask coating material just like PMMA does in the current e-beam lithographic technique. Here, the etching action will be exerted by a layer of excess protons localized at the water-substrate interface according to the proton electrostatic localization theory. With the use of protonic “resist” masks, only part of the substrate surface that is not protected by a protonic “resist” mask will be etched. In this way, many proton-etching patterns such as the word “ODU” and/or any other patterns like a round disk pattern (FIG. 6P_{I}) may be created on a substrate. One of the advantages is that this method uses just water with DC electrodes operated with nearly an open-circuit water electrolysis process (with minimal electricity energy consumption), and no additional chemicals such as salts, nitric acid or chloric acid are needed here.

Since the protonic treatment (such as the protonation of synthetic substrate materials such as (poly)aniline or proton-etching of micro/nanometer materials) can be operated in a pure water environment with neutral bulk pH, when the so-treated substrate is taken out of the chamber, it may immediately emerge as a quality product with the cleanness of pure water without requiring any additional washing/cleaning step that a conventional acid-treatment process would require. Thus, the method disclosed here in accordance with one of the various embodiments of the invention represents a remarkably clean “green chemistry” technology.

In addition, as illustrated in FIG. 1b, the present invention in one of the various embodiments has created and demonstrated the formation of a localized excess protons layer at the water-membrane interface in an anode water-membrane-water cathode system, where excess protons were generated by water electrolysis in an anode electrode chamber while excess hydroxyl anions were created in a cathode chamber. When a positive voltage is applied to the anode electrode in water, it first attracts the hydroxyl anions to anode electrode surface and then some of counter ions (protons) distribute themselves near the anions layer, forming a typical “electric double layer” on the anode surface (FIG. 1b, right side). When a significant number of excess protons are produced by water electrolysis (in mimicking a biological proton production process such as the respiratory electron-transport-coupled proton pumping system and the photosynthetic water-splitting process) in the anode chamber, the excess protons electrostatically distribute themselves at the water-surface (including the membrane surface) interface around the water body (including a part of the “electric double layer” at the anode surface). From here, it can be seen that the excess proton layer formed at the water-membrane interface is apparently a type of special extension from the secondary (proton) layer of the “electric double layer” at the anode. The excess proton layer at the water-membrane interface electrostatically attracts the excess hydroxyl anions in the cathode chamber at the other side of the member, forming an excess anions-membrane-excess proton capacitor-like structure.

Since the membrane is an insulator layer (not an electrode), the excess proton layer at the water-membrane interface is likely to be a special monolayer (with a thickness probably of about 1 nm), but definitely not an “electric double layer”. This novel feature of being an excess proton monolayer at the membrane-interface is also consistent with the fundamental understanding of the “electric double layer” theory since the excess proton layer created and demonstrated here can be understood as a kind of special extension from the second (proton) layer of the anode's “electric double layer” (FIG. 1b, right side) around the proton-conductive water body.

When the electrolysis voltage is turned off, the electrical polarization at both anode and cathode disappears and so does the “electric double layer”, leaving only the excess proton layer around the anode chamber water body and the similarly formed excess hydroxyl (anions) layer around the cathode chamber water body as illustrated in FIG. 1c. The excess anions-membrane-excess proton capacitor (shown in the middle of FIG. 1c) may represent a proof-of-principle mimic for an energized biological membrane such as a mitochondrial membrane system at its energized resting state.

The excess protons created and demonstrated experimentally here have special features. Unlike a charge-balanced (1,1) electrolyte, excess protons do not have counter ions since their counter ions, the excess anions, are on the other side of the membrane as shown in FIG. 1c. Therefore, the common “electric double layer” models (McLaughlin, 1989 Annual Review of Biophysics and Biophysical Chemistry, 18:113-136) including the Gouy-Chapman theory and Debye shielding length concept may not necessarily be used as an accurate description for the excess proton layer that has now been experimentally demonstrated here in accordance with one of the various embodiments of the present invention.

The Debye shielding length concept is commonly used to estimate the thickness of an electric double layer. It however may not be able to accurately estimate the thickness of this special excess proton monolayer demonstrated in the invention. Since both the “electric double layer” models (including the Gouy-Chapman theory) and the Debye shielding length concept are based on charge-balanced electrolytes with cations and anions being together in the same water body, they may not be applicable to the special excess protons that do not have counter ions in their associated water body since their counter ions (excess hydroxyl anions) are completely in a separated water body on the other side of the membrane as illustrated in FIG. 1c.

Furthermore, the excess protons created and demonstrated experimentally here are fundamentally different from the protons that are attracted to the biological membrane surface by the membrane's fixed surface charges such as the negatively-charged phosphate groups of a typical biological membrane that may attract protons and other cations to its surface forming an electric double layer along the membrane negatively charged surface as expected by the Gouy-Chapman theory. That type of membrane surface charge-associated electric double layer (associated with the “surface potentials”) always exists even when the protonic motive force (pmf) is completely zero. Therefore, the membrane surface potentials-attracted protons do not contribute to the protonic motive force that drives the flow of protons across the membrane for ATP synthesis as pointed out also in bioenergetics textbooks (Nicholls & Ferguson 2013 Bioenergetics, 27-51, Academic Press). In contrast, the excess protons can electrostatically localize themselves to the water-membrane interface without requiring any membrane surface charges, which are fundamentally different from those charge-balanced protons attracted by the membrane surface potentials. The concept of excess protons, however, is not to be confused with the commonly known charge-balanced protons in water and biological systems.

The creation of an excess protons layer has recently been experimentally demonstrated at a water-membrane interface in an anode water-membrane-water cathode system using a charge-neutral and inert membrane such as a Teflon membrane in mimicking the biological systems (Saeed and Lee 2015 Bioenergetics 4: 127. doi:10.4172/2167-7662.1000127). In fact, it is this type of free excess protons that have the dynamic properties to be coupled to ATP synthase that are relevant to the protonic motive force in biological systems.

Therefore, the excess protons layer demonstrated through the experiments represents an advance having scientific and technological implications. For example, the excess protons layer may be employed as a special tool to enable the extraction of environmental heat molecular thermal motion energy to create additional protonic motive force (equivalent to Gibbs free energy) to do useful work as described herein.

According to one of the preferred embodiments, it is a preferred practice to use well-degassed liquid water that does not contain too much dissolved gases for the creation of excess protons layer at a membrane-water interface. For example, during the winter season when the laboratory temperature (typically about 22° C.) is significantly higher than the outside water supplying sources, the Millipore (filtered) water made from such a cold air-saturated water source often contains too much dissolved air gases that may slowly release the excess gases due to gas solubility change in response to temperature changes, forming numerous tiny gas bubbles on the surfaces of water chambers including the Al-Tf-Al membrane surface as was observed in one of the experiments. These tiny gas bubbles can sometimes become so problematic that they could negatively affect the formation and detection of localized protons on the Al-Tf-Al membrane surface because the gas bubbles apparently reside at the water-membrane interface and form an air-gap barrier between the membrane and the liquid water phase. For example, to eliminate this problem for improved reproducibility of the experiments, a special effort was made on the laboratory water source: the Millipore water was degassed by boiling the water through autoclave and then cooled down to room temperature before the experimental use.

Degassing of liquid water can be quickly accomplished also by use of a vacuum pump in conjunction with sonication of the liquid water. With the degassed liquid water, generation of an excess protons layer at a membrane-water interface has been experimentally demonstrated with high reproducibility. Alternatively, degassing can be accomplished by letting the liquid water to fully equilibrate with the laboratory temperature and air conditions for more than 10 days, during which liquid water can naturally (slowly) release the excess dissolved gases towards equilibration. Use of fully equilibrated liquid water which no longer generates any gas bubbles on membrane surface also produced good reproducible results.

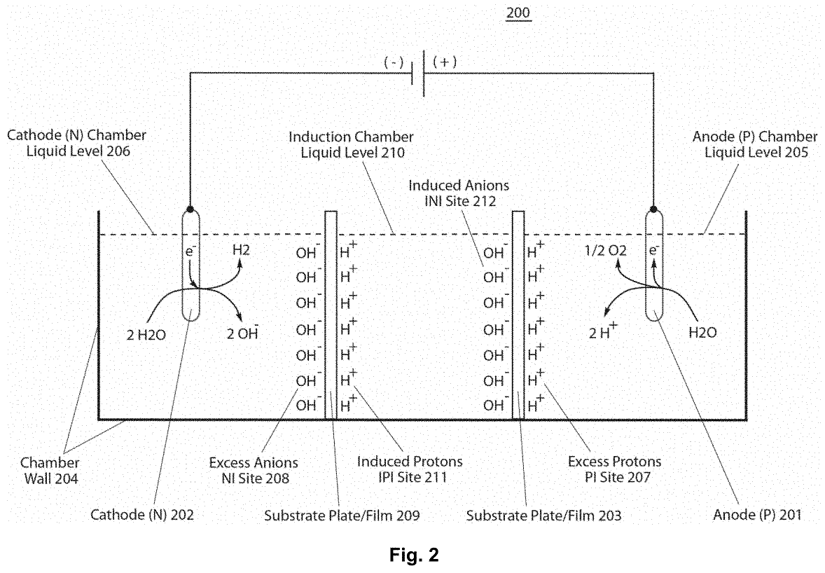

Referring to FIG. 2, an excess proton production system 200 with three chambers is illustrated. The excess proton production system includes a substrate plate/film 203 placed in between an anode chamber 205 and an induction chamber 210 at the middle and another substrate plate/film 209 placed in between the induction chamber 210 and a cathode chamber 206. The chamber wall 204 is made of water- and proton-impermeable and chemically-inert materials such as Teflon, plastic material, and glass, which are unreactive even if under high power voltage. The substrate plate/films (membranes) 203 and 206 joins with the chamber wall 204 using water-tight seal, resulting in the three separate chambers: the anode water chamber 205, the induction chamber 210, and the cathode water chamber 206. That is, the induction chamber 210 is formed in between the two substrate plate/films 203 and 209 which serve as its two end walls in conjunction with the wall 204 as its bottom and side walls.

According to one of the various embodiments, all the three chambers (from the left to the right: the cathode chamber, the induction chamber, and the anode chamber) are filled with pure water as shown in FIG. 2. The anode (N) chamber liquid level 205 is preferably set at the same level as the cathode (N) chamber liquid level 206 and at the induction chamber liquid level 210. Both the anode (P) 201 and cathode (N) 202 are typically made of stable electrode materials such as metallic platinum, palladium, gold, copper, stainless steel, graphite, and/or micro/nanometer carbon fibers. The excess protons and excess hydroxyl anions are generated in the anode and cathode water bodies through the use of “open-circuit” water-electrolysis by applying a direct current (DC) voltage across the anode (P) 201 and cathode (N) 202 electrodes. In accordance of the present invention, the excess protons produced by the “open-circuit” water-electrolysis process typically localize at the water-substrate interface (PI Site 207) along the surface of the plate/film (or membrane) where they electrostatically induce hydroxyl anions at the other side (INI Site 212) of the substrate plate, forming an “excess protons-plate-excess anions” capacitor-like structure. Similarly, the excess hydroxyl anions produced by the “open-circuit” water-electrolysis process localize at the water-substrate interface (NI Site 208) along the surface of the plate/film (or membrane) where they induce protons at the other side (IPI Site 211) of the substrate plate, forming another “excess protons-plate-excess anions” capacitor-like structure.

According to one of the various embodiments, the excess proton production system 200 (FIG. 2) can be operated in a manner similar to that of the excess proton production system 100 (FIG. 1) except that it can simultaneously treat two substrate plate/films 203 and 209.

Furthermore, according to one of the various embodiments, when necessary, certain chemicals such as sodium bicarbonate and potassium bicarbonate may be added into the induction chamber 210 to modulate (reduce) the effective concentration of the induced protons at the IPI site 211 by Na⁺ (or K⁺) cation exchange with the localized protons at the IPI site 211 to achieve more desirable results. In this way, the anode (P) chamber 205 and the cathode (N) chamber 206 can still work with pure water for production of excess protons and hydroxyl anions through the “open-circuit” water electrolysis process without the presence of any added chemicals that may interfere with the process.

The effective concentration of the electrostatically localized protons at the equilibrium of cation exchange can be calculated as:

$$[ {H}_{L}^{+} ] = \frac{{[ {H}_{L}^{+} ]}^{0}}{{\prod }_{i = 1}^{n} \{ {K}_{Pi} ( \frac{[ {M}_{pB}^{i +} ]}{[ {H}_{pB}^{+} ]} ) + 1 \}} \qquad [ 6 ]$$

where [H_{L}⁺]⁰ is the ideal effective concentration of localized protons without cation exchange. Here, K_{Pi} is the equilibrium constant for non-proton cations (Mⁱ⁺) to exchange for the localized protons at the water-membrane interface; [M_{pB}ⁱ⁺] is the concentration of the non-proton cations in the induction chamber liquid medium; and [H_{pB}⁺] is the proton concentration in the bulk phase of the induction chamber liquid medium.

Since protons have the smallest atomistic diameter and can exist as part of the water molecules, they can electrostatically distribute themselves to the water-membrane interface much more favorably than any other cations, such as Na⁺, Mg⁺⁺ or K⁺. Therefore, the equilibrium constant for protons to electrostatically occupy the cation sites at the water-membrane interface (in any possible competition with any other cations) is expected to be much larger than one. Certain cation exchange experimental studies (Lee et al., 2010 Environmental Science & Technology, 44(20):7970-7974; Skjemstad et al., 2008 Communications in Soil Science and Plant Analysis, 39(5-6):926-937) have recently indicated that the equilibrium constant for protons to exchange with other cations for cation binding sites can be on the order of 4.7×10⁺⁶. Conversely, the equilibrium constant K_{Pi} for non-proton cations to delocalize the localized protons from the membrane-water interface may be in the order of around 2.1×10⁻⁷. Use of the cation exchange equilibrium constant K_{Pi} can calculate the effective concentration of the localized excess protons using Equation 6 when non-proton cations are present, which is a parameter that may be helpful also to certain practitioners in accordance of the present invention.

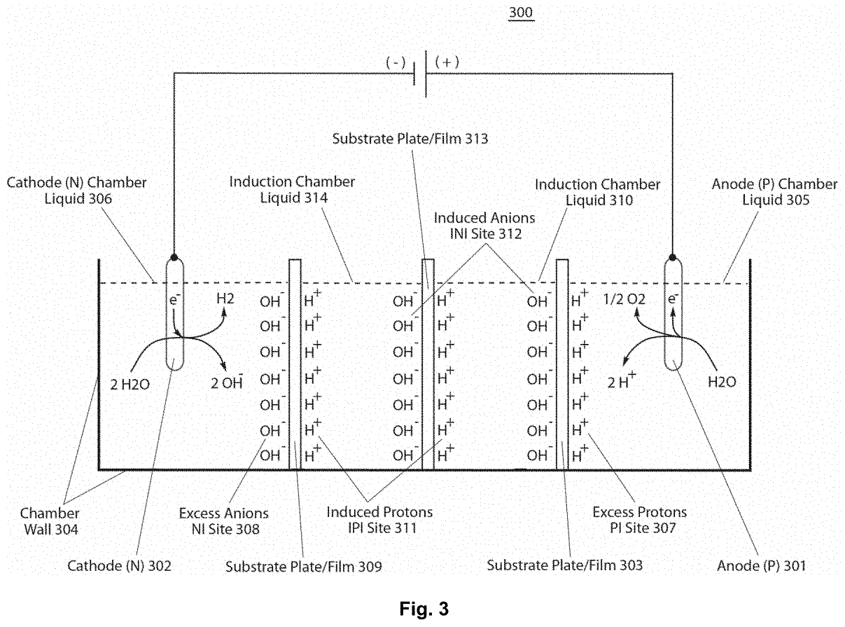

Referring to FIG. 3, a four-chamber excess proton production and utilization system 300 is illustrated. The four-chamber system 300 includes three substrate plate/films 303, 309, and 313 that are placed in between an anode chamber 305 and a cathode chamber 305, forming additional two induction chambers 310 and 314 among the three substrate plate/films 303, 309, and 313. The chamber wall 304 is made of water-proton-impermeable and chemically-inert materials such as Teflon, plastic material, and glass, which are unreactive even if under high power voltage.

According to one of the various embodiments, all the four chambers (from the left to the right: the cathode chamber, the induction chambers 310 and 314, and the anode chamber) are filled with pure water as shown in FIG. 3. The anode (N) chamber liquid level 305 is set preferably at the same level as the cathode (N) chamber liquid level 306 and also at the same level as the induction chamber liquid level 314 and 310. Both the anode (P) 301 and cathode (N) 302 are typically made of stable electrode materials such as metallic platinum, palladium, gold, copper, certain stainless steel, graphite, micro/nanometer carbon fibers, and/or combination thereof. The excess protons and excess hydroxyl anions are generated in the anode and cathode water bodies through the use of “open-circuit” water-electrolysis by applying a direct current (DC) voltage across the anode (P) 301 and cathode (N) 302 electrodes (FIG. 3). The excess protons produced by the “open-circuit” water-electrolysis process localize at the water-substrate interface (PI Site 307) along the surface of the plate/film (or membrane) where they induce hydroxyl anions at the other side (INI Site 312) of the substrate plate, forming an “excess protons-plate-excess anions” capacitor-like structure. Similarly, the excess hydroxyl anions produced by the “open-circuit” water-electrolysis process localize at the water-substrate interface (NI Site 308) along the surface of the plate/film (or membrane) where they induce protons at the other side (IPI Site 311) of the substrate plate. As a result, two additional “excess protons-plate-excess anions” capacitor-like structures are formed.

According to one of the various embodiments, the excess proton production and utilization system 300 (FIG. 3) can be operated in a manner similar to that of the system 100 (FIG. 1) except that it can simultaneously treat three substrate plate/films 303, 209 and 313. Furthermore, certain chemicals such as sodium bicarbonate and potassium bicarbonate may be added into the induction chamber liquid 310 and 314 to modulate (reduce) the effective concentration of the induced protons at the IPI site(s) 311 by Na⁺ (or K⁺) cation exchange with the localized protons at the IPI site(s) 311 when such a modulation adjustment may become desirable in certain special applications.

According to one of the various embodiments, many more induction chambers can be used in between the anode chamber and the cathode chamber to simultaneously treat many substrate plate/films in a single system like the system 300 (FIG. 3). The number of induction chambers that can be used in between an anode chamber and a cathode chamber in a single excess protons and hydroxyl anions production-utilization system is selected from the group consisting of 0, 1, 2, 3, 4, 5, 6, 7, 8, 10, 11, 12, 13, 14, 15, 17, 18, 19, 20, 21, 22, 23, 24, 25, 26, 27, 28, 29, 30, 40, 50, 60, 70, 90, 100 and more. Consequently, the number of substrate plate/films that can be simultaneously treated in a single excess protons and hydroxyl anions production and utilization system is selected from the group consisting of 1, 2, 3, 4, 5, 6, 7, 8, 10, 11, 12, 13, 14, 15, 17, 18, 19, 20, 21, 22, 23, 24, 25, 26, 27, 28, 29, 30, 31, 41, 51, 61, 71, 81, 91, 101, and more. These selections may be used in part and/or in any combinations depending on the value of water electrolysis voltage and the effective concentration of localized excess protons that may be required for a given processing treatment application.

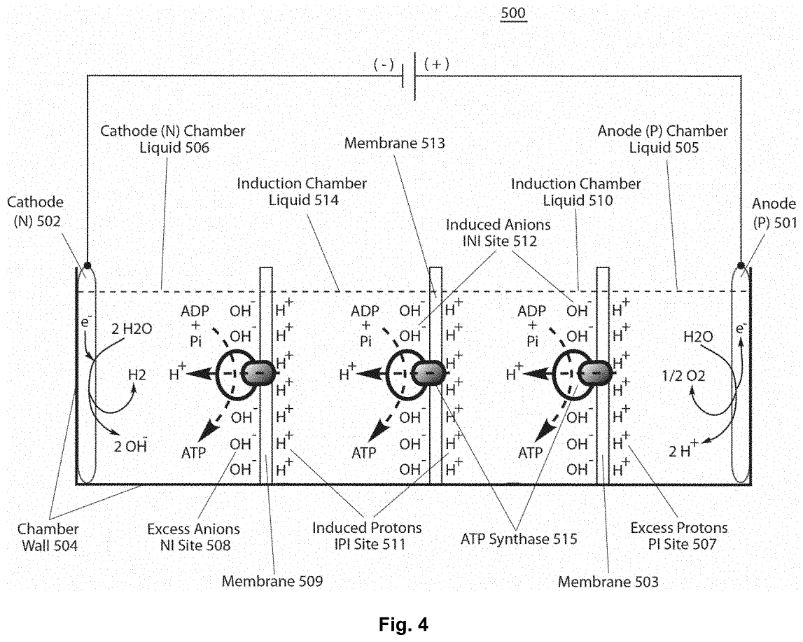

Referring to FIG. 4, a multi-chamber excess proton production and utilization system 500 is illustrated. The multi-chamber system 500 comprises: three (more or multiple) membranes 503, 509, and 513 that are placed in between an anode chamber 505 and a cathode chamber 506, forming additional two (more or multiple) induction chambers 510 and 514 among the three (more or multiple) membranes 503, 509, and 513. The chamber wall 504 is made of water- and proton-impermeable and chemically-inert materials such as Teflon, plastic material, and glass, which are unreactive even if under high power voltage. There are proton users such as ATP synthase 515 that are embedded with each of the three (more or multiple) membranes 503, 509, and 513 that mimic a biological membrane.

According to one of the various embodiments, the “excess protons-membrane-hydroxyl anions” capacitor-like structures may be employed to enable novel utilization of low-grade heat energy from the ambient temperature environment such as the environmental heat energy associated with the molecular thermal motion of the localized protons to perform useful work such as driving the synthesis of ATP (FIG. 4). Typically, this special energy technology process includes the following steps and features: a) through use of the “open-circuit” water-electrolysis process, excess protons are generated in anode liquid chamber 505 while excess hydroxyl anions are created in cathode liquid chamber 506; b) the generated excess protons electrostatically localize themselves primarily along the water-membrane interface at the PI site 507 while the excess anions electrostatically localize themselves primarily along the water-membrane interface at the NI site 508; c) the excess protons at PI site 507 in conjunction with the excess anions at NI site 508 electrostatically induce the formation of the induced anions at INI site(s) 512 and the induced protons at IPI site(s) 511 in the induction liquid chambers 510 and 514; d) The formation of the electrostatically localized protons layer constitutes a type of “negative entropy” event during the formation of multiple “localized protons-membrane-anions” capacitor-like structures; e) the formation of multiple “localized protons-membrane-anions” capacitor-like structures results in the formation of membrane potential across each of the membranes; f) In addition to the generation of membrane potential, significant amount of “bonus” local proton motive force (useful Gibbs free energy) is created also from the “entropy effect” of the localized protons since their thermal motions (latent heat) possibly including their Brownian motion can drive nanometer-scale molecular machines such as F₀F₁-ATP synthase embedded in the membrane; g) Utilization of the total protonic motive force (the membrane potential and the local proton motive force) from the localized protons at PI site and IPI site(s) to do work as the protons flow across each of the membranes through the membrane-embedded ATP synthase 515 in driving ATP synthesis from ADP and Pi (inorganic phosphate); and h) the molecular hydrogen (H2) and oxygen (O2) gas products are collected at the cathode and the anode, respectively.

According to one of the various embodiments, it is a preferred practice to place the proton-generating anode electrode well into the bulk phase liquid and to keep the mouths of proton users such as ATP synthase 515 being located rightly within the localized excess protons layer along the membrane surface as illustrated in FIG. 4, for the best effect to utilize environmental heat associated with the thermal motion energy of localized protons to perform useful work such as driving the synthesis of ATP in accordance with the present invention.

The well-established scientific knowledge that protonic motive force (pmf) is equivalent to Gibbs free energy (ΔG=−n. F. pmf, where n is proton charge and F is Faraday constant) that can be employed to do useful work as in the example of driving ATP synthesis is one of the fundamentals in the invention. It is known that ATP represents a form of chemical energy that can be used not only in living organisms but also in certain industrial biochemical engineering processes for making certain biomolecules such as nucleic acids and other compounds of importance including certain pharmaceutical-related products.

According to one of the various embodiments, the total protonic motive force (pmf) across a biological membrane and/or a bio-inspired synthetic membrane taking into account the surface localized protons can be expressed as

$$pmf = Δ ψ + \frac{2.3 RT}{F} {log}_{10} ( \{ [ {H}_{L}^{+} ] + [ {H}_{pB}^{+} ] \} / [ {H}_{nB}^{+} ] ) \qquad [ 7 ]$$

Here Δψ is the electrical potential difference across the membrane, R is the gas constant, T is the absolute temperature, F is Faraday's constant, [H_{L}⁺] is the concentration of surface localized protons, [H_{pB}⁺] is the proton concentration in the periplasmic bulk aqueous phase (equivalent to the anodic chamber liquid of FIG. 1c), and [H_{nB}⁺] is the proton concentration in the cytoplasmic bulk phase (equivalent to the cathodic chamber liquid in FIG. 1c).

This pmf expression may be rewritten to isolate the environmental heat thermal molecular motion energy contribution due to the localized protons as follows:

$$pmf = Δ ψ + \frac{2.3 RT}{F} {log}_{10} ( [ {H}_{pB}^{+} ] / [ {H}_{nB}^{+} ] ) + \frac{2.3 RT}{F} {log}_{10} ( 1 + [ {H}_{L}^{+} ] / [ {H}_{pB}^{+} ] ) \qquad [ 8 ]$$

The first two terms of Eq. 8 comprise the “classic” expression for the protonic motive force (pmf) used in textbooks (Nicholls and Ferguson 2013, Bioenergetics (Fourth Edition), Academic Press: Boston. p. 27-51; Skulachev, Bogachev, and Kasparinsky 2012, Principles of Bioenergetics, Springer: Berlin Heidelberg) and the third term is the local pmf component from the localized protons that may be employed as a special tool to extract thermal molecular motion energy (environmental heat) to create useful Gibbs free energy to do work according to one of the various embodiments.

For certain industrial applications, the bulk phase liquid pH (i.e., the bulk liquid phase proton concentrations) can be set to be the same. For example, a liquid medium such as pure water (pH 7.0), air-equilibrated water (pH 5.8) or a pH-buffered reaction medium can be used at the same pH for each of the all liquid chambers as shown in the example of FIG. 4. According to one of the various embodiments, the creation of excess protons does not significantly alter the bulk liquid phase proton concentration in any of the liquid chambers since excess protons do not stay in the bulk liquid phase and they electrostatically localize primarily at the water-membrane interface associated with the dominant capacitance there. This prediction has now been verified experimentally by the measurements of the bulk liquid phase pH and by the detection of the localized protons detection with proton-sensing Al films. Therefore, under this special condition, the second term of Eq. 8 can be practically treated as zero and the total pmf value may be calculated practically by using of the first term (membrane potential) and the third term (local pmf).

For some special industrial applications, certain salt solutions and/or buffer solutions may be employed in any of the liquid chambers (as shown in FIG. 4) to modulate the total pmf values. The effective concentration of the electrostatically localized protons at the equilibrium of cation exchange can be calculated according to Eq. 6, which may then be used in calculating the pmf value with Eq. 8.

Table 2 lists the exemplary pmf values calculated using Eqs. 6-8 across a mimicked biological membrane with a specific membrane capacitance per unit surface area (C/S) of 13.2 mf/m² and a reasonable thinness of the localized proton layer (l) of 1 nm with an exemplary physiological liquid medium. The exemplary physiological liquid medium comprises the following cations: 300 mM Na⁺, 3.584 mM K⁺, 0.1 mM Mg⁺⁺, 0.4557 mM Ca⁺⁺, 38.08 μM Zn⁺⁺, 25.17 μM Fe⁺⁺, 5.557 μM Mn⁺⁺, 1.602 μM Cu⁺⁺, 0.859 μM Co⁺⁺, and 0.971 μM NH₄⁺. The equilibrium constants K_{Pi} of cation exchange with localized protons used in this calculation were estimated from preliminary experimental data: 7.41×10⁻⁸ and 2.48×10⁻⁸ for Na⁺ and K⁺. The average of these two (4.95×10⁻⁸) was used to estimate for K_{Pi} of the other monovalent cation NH₄⁺. The K_{Pi} value of 2.1×10⁻⁷ for divalent cation Mg⁺⁺ was calculated from the experimental data of cation exchange studies (Lee et al. 2010 Environmental Science & Technology, 44(20): 7970-7974; Skjemstad et al., 2008 Communications in Soil Science and Plant Analysis, 39(5-6): 926-937) and was used for the other divalent cations here as well.

The results listed in Table 2 demonstrate that the “local” pmf (equivalent to Gibbs free energy) extracted isothermally from the environmental heat with localized protons as calculated from the third term of Eq. 8 is a very significant component of the total pmf. With a membrane potential of 50 mV and liquid bulk phase pH 7, the “local” pmf extracted from the environmental heat with localized protons is 280 mV, which represents nearly 85% of the total pmf (330 mV). Similarly, with a membrane potential of 25 mV and liquid bulk phase pH 7, the “local” pmf extracted from environmental heat with the localized protons is 263 mV, which represents as much as 91% of the total pmf (288 mV) and is more than sufficient to drive ATP synthesis that requires a minimal pmf of 116 mV. Therefore, these results demonstrate that the innovative application of localized excess protons in accordance with the invention may provide a special novel energy technology process function to isothermally extract environmental heat including the molecular thermal motion energy associated with localized protons at ambient environmental temperature for generating local protonic motive force (equivalent to Gibbs free energy) to do useful work such as driving ATP synthesis.

TABLE 2
Calculated pmf values across a mimicked biological membrane with a specific
membrane capacitance per unit surface area (C/S) of 13.2 mf /m² and a reasonable thinness
of the localized proton layer (l) of 1 nm under a simulated physiological salt solution using
Eqs. 6-8 at the temperature T = 298K. The “local” pmf is the last term in Eq. 8 due to the
localized protons, while the first two terms of Eq. 8 give the “classic” pmf.

| | | | | Exchange | | Local | Classic | Total |
|---|---|---|---|---|---|---|---|---|
| | | Δψ | [H_{L}⁺]⁰ | reduction | [H_{L}⁺] | pmf | pmf | pmf |
| pH_{pB} | pH_{nB} | (mV) | (molar) | factor | (molar) | (mV) | (mV) | (mV) |
| 8.2 | 8.2 | 25 | 3.42 × 10⁻³ | 4.683 | 7.30 × 10⁻⁴ | 299 | 25 | 324 |
| 8.2 | 8.2 | 50 | 6.84 × 10⁻³ | 4.683 | 1.46 × 10⁻³ | 317 | 50 | 367 |
| 8.2 | 8.2 | 100 | 1.37 × 10⁻² | 4.683 | 2.92 × 10⁻³ | 335 | 100 | 435 |
| 8.2 | 8.2 | 150 | 2.05 × 10⁻² | 4.683 | 4.38 × 10⁻³ | 345 | 150 | 495 |
| 8.2 | 8.2 | 200 | 2.74 × 10⁻² | 4.683 | 5.48 × 10⁻³ | 352 | 200 | 552 |
| 7.0 | 7.0 | 200 | 2.74 × 10⁻² | 1.225 | 2.23 × 10⁻² | 316 | 200 | 516 |
| 7.0 | 7.0 | 150 | 2.05 × 10⁻² | 1.225 | 1.68 × 10⁻² | 309 | 150 | 459 |
| 7.0 | 7.0 | 100 | 1.37 × 10⁻² | 1.225 | 1.12 × 10⁻² | 298 | 100 | 398 |
| 7.0 | 7.0 | 50 | 6.84 × 10⁻³ | 1.225 | 5.58 × 10⁻³ | 280 | 50 | 330 |
| 7.0 | 7.0 | 25 | 3.42 × 10⁻³ | 1.225 | 2.79 × 10⁻³ | 263 | 25 | 288 |
| 5.8 | 5.8 | 25 | 3.42 × 10⁻³ | 1.014 | 3.37 × 10⁻³ | 197 | 25 | 222 |
| 5.8 | 5.8 | 50 | 6.84 × 10⁻³ | 1.014 | 6.73 × 10⁻³ | 214 | 50 | 264 |
| 5.8 | 5.8 | 100 | 1.37 × 10⁻² | 1.014 | 1.35 × 10⁻² | 232 | 100 | 332 |
| 5.8 | 5.8 | 150 | 2.05 × 10⁻² | 1.014 | 2.02 × 10⁻² | 243 | 150 | 393 |
| 5.8 | 5.8 | 200 | 2.74 × 10⁻² | 1.014 | 2.70 × 10⁻² | 250 | 200 | 450 |

The results shown in Table 2 (the “local” pmf of 263 mV extracted isothermally from environmental heat with localized protons with a membrane potential of 25 mV) can also help to elucidate the mystery of how a hyperthermophilic archaeon (Thermococcus onnurineus NA1) could grow by the anaerobic oxidation of formate to CO2 and H2, which has very little free energy change at its physiological conditions (ΔG⁰=−2.6 kJ/mol) (Lim et al., 2014 Proceedings of the National Academy of Sciences, USA 111(31):11497-11502). If this free energy (ΔG⁰=−2.6 kJ/mol) is utilized to drive formation of an electrochemical proton gradient across the membrane, it could possibly form a membrane potential of about 25 mV, which, if based on the delocalized proton view of Peter Mitchell's Chemiosmotic Theory, would translate to a classic pmf of only 25 mV that would not be sufficient to drive ATP synthesis to support cell growth. On the other hand, based on the data presented in Table 2 of the invention, a membrane potential of 25 mV may translate to a total pmf of 288 mV with a local pmf (263 mV) generated from environmental heat molecular motion energy of the localized protons, which is sufficient to drive ATP synthesis to support cell growth (possibly also involving a Na⁺/H⁺ antiporter in the cell). Therefore, that difficult bioenergetics question associated with Thermococcus onnurineus NA1 may now be answered satisfactorily by the special energy-transduction mechanism of localized protons in extracting environmental heat molecular motion energy to generate a local pmf as much as 263 mV as disclosed herein.

The data in Table 2 also show that at a membrane potential of 200 mV with the same pH neutral liquid media, the “local” pmf extracted from environmental heat is 316 mV which is 61% of the total pmf (516 mV). This result indicates that at a high membrane potential (200 mV), its effect on enhancing “local” pmf can become limited. Therefore, according to one of the various embodiments, it is a preferred practice to employ a relatively smaller membrane potential as long as it can still electrostatically hold the excess protons at the liquid membrane interface to isothermally extract the environmental heat energy to generate the “local” pmf, yielding a better ratio of local pmf to total pmf.

According to one of the various embodiments, the special energy technology process for generating useful Gibbs free energy from utilization of molecular thermal motion energy associated with localized protons has a special feature that its local protonic motive force (pmf) generated from its isothermal utilization of environmental heat energy may be calculated according to the following formula:

$$Local pmf = \frac{2.3 RT}{F} {log}_{10} ( 1 + [ {H}_{L}^{+} ] / [ {H}_{pB}^{+} ] ) \qquad [ 9 ]$$

Where R is the gas constant, T is the absolute temperature, F is Faraday's constant, [H_{L}⁺] is the concentration of surface localized protons, and [H_{pB}⁺] is the proton concentration in the anode bulk aqueous phase.

With this Equation [9], it is now, for the first time, clearly expressed that the local pmf is a logarithmic function of the ratio of localized proton concentration [H_{L}⁺] at the liquid-membrane interface to the delocalized proton concentration [H_{pB}⁺] in the liquid bulk phase at the same side of the membrane (but not to the delocalized proton concentration [H_{nB}⁺] in the cathodic chamber liquid at the other side of the membrane). It is the electrostatic proton localization that brings the excess protons to the water-membrane interface that enables the isothermal utilization of molecular thermal motion energy from the ambient temperature environment to create protonic motive force without being constrained by the Second Law of Thermodynamics. Therefore, this also represents a breakthrough in the fundamental understanding of energy transduction and energy renewal and utilization, which may have seminal scientific and practical implications for energy and environmental sustainability on Earth.

Furthermore, from Eq. 9 in conjunction with Eq. 6, it is understood that when a significant amount of cations such as Na⁺ occupy the localized proton layer by cation exchange as in the case with high salt concentrations, it may form a localized sodium ion (Na⁺) concentration [Na_{L}⁺] while reducing the concentration of localized protons [H_{L}⁺]. Consequently, in the presence of high sodium ion (Na⁺) concentration [Na_{pB}⁺] in liquid bulk phase, certain amounts of local pmf may be converted to local sodium motive force (smf) through cation exchange with the localized protons. The value of local smf may be calculated as:

$$Local smf = \frac{2.3 RT}{F} {log}_{10} ( 1 + [ {Na}_{L}^{+} ] / [ {Na}_{pB}^{+} ] ) \qquad [ 10 ]$$

Therefore, according to one of the various embodiments, application of excess protons in the presence of high sodium cation (Na⁺) concentration [Na_{pB}⁺] in liquid bulk phase may be used to generate local smf also from the special utilization of latent heat energy from the ambient temperature environment to do useful work such as driving an Al Ao-ATP Synthase for ATP Synthesis (McMillan et al., 2011 Journal of Biological Chemistry, 286(46):39882-39892). Therefore, exemplary embodiments may be extended to other localizable cations and other species such as Na⁺, K⁺, Li⁺, Rb⁺, Cs⁺, Co⁺⁺, Ni⁺⁺, Zn⁺⁺, Cu⁺⁺, Fe⁺⁺, Mn⁺⁺, Ca⁺⁺, and/or Mg⁺⁺ for various industrial applications including the special extraction of environmental heat molecular thermal motion energy for energy technology applications.

According to one of the various embodiments, it is a preferred practice to employ multiple membranes, of which each is with a relatively smaller membrane potential, in a multi-chamber system such as that illustrated in FIG. 4 that can be employed with use of a relatively small electrolysis voltage for generating excess protons to extract environmental heat molecular thermal motion energy to create a total pmf value much larger than the input electrolysis voltage. The extracted molecular thermal motion energy in the form of pmf from the environmental heat of ambient temperature environment may be utilized to drive nanometer machines such as ATP synthase, proton-driven molecular transport systems and enzymes to perform useful work.

According to one of the various embodiments, depending on a given specific application and its associated temperature conditions, liquid media compositions, and the properties of proton users and membrane material such as its thickness, proton capacitance and other physical chemistry properties, the number of membranes that may be used per multi-chamber system as illustrated in FIG. 4 for the purpose of extracting environmental heat to create local pmf may be selected from the group consisting of 1, 2, 3, 4, 5, 6, 7, 8, 8, 9, 10, 20, 30, 40, 50, 60, 70, 80, 90, 100, 200, 300, 500, 1000, 2000, 5000, 10,000, 100,000, 1,000,000, more and/or within a range bounded by any two of these values.

According to one of the various embodiments, depending on a given specific application and its associated temperature conditions, liquid media compositions, and the properties of proton users and the membrane material such as its thickness, proton capacitance and other physical chemistry properties, the membrane potential for the purpose of extracting environmental heat to create local pmf may be selected from the group consisting of 0.1 mV, 0.5 mV, 1 mV, 5 mV, 10 mV, 15 mV, 20 mV, 25 mV, 30 mV, 40 mV, 50 mV, 60 mV, 70 mV, 80 mV, 90 mV, 100 mV, 110 mV, 120 mV, 130 mV, 140 mV, 150 mV, 200 mV, 250 mV, 300 mV, 500 mV, 1000 mV, 2 V, 5V, 10 V, 20 V, 50 V, 100 V, 200 V, 300 V, 500V, 1000V, and/or within a range bounded by any two of these values.

According to one of the various embodiments, depending on a given specific application and its associated temperature conditions, liquid media compositions, and the properties of proton users and the membrane material such as its thickness, proton capacitance and other physical chemistry properties, the said special energy renewal technology process to isothermally extract environmental heat molecular thermal motion energy associated with localized protons for generating local protonic motive force (equivalent to Gibbs free energy) may be operated in a wide range of temperatures including ambient temperatures, elevated temperatures, and/or low temperatures.

The results listed in Table 2 showed that the “local” pmf extracted from the environmental heat with localized protons at neutral pH or slightly alkaline bulk liquid can be somewhat bigger than that at acidic conditions at the same membrane potential and liquid media ionic strength. For example, at the membrane potential of 100 mV, the amounts of “local” pmf with liquid bulk phase pH 8.2 and 7.0 are 335 and 298 mV, both are bigger than that (232 mV) with liquid bulk phase pH 5.8. Therefore, according to one of the various embodiments, it is a preferred practice to employ neutral or slightly alkaline bulk liquid pH to better generate the “local” pmf (Gibbs free energy).

As shown in Table 2, the liquid media with pH 5.8, 7.0, and 8.2 practically all work very well with the excess protons-based energy technology to generate “local” pmf in utilizing the environmental heat energy which is conventionally thought as impossible to be used from ambient temperature environment. Depending on a given specific application and its associated temperature conditions, liquid media compositions including the ionic strength, the properties of proton users, the membrane material such as its thickness, proton capacitance and other physical chemistry properties, the pH of liquid media may however be from selected the group consisting of pH 1, 2, 3, 4, 5, 6, 6.5, 7.0, 7.5, 8.0, 8.5, 9.0, 9.5, 10, 10.5, 11, 11.5, 12, 13, 14 and/or within a range bounded by any two of these values in accordance with one of the various embodiments of the present invention.

Meanwhile, the data also indicate that at liquid bulk phase pH 8.2, the exchange reduction factor (4.683) gets significantly bigger than those (1.225 and 1.014) with bulk liquid pH 7.0 and 5.8, which could negatively impact the localized proton concentration. Therefore, according to one of the various embodiments, it is also a preferred practice to employ pure deionized water or low salt liquid media to more effectively generate “local” pmf (equivalent to Gibbs free energy) from environmental heat molecular motion energy, although high salt solution can also be employed when the liquid pH is not high such as above pH 12.

One of the key fundamental features in the invention is the utilization of environmental heat with localized protons to recycle/utilize the fully dissipated waste heat energy in the environment, which conventionally is thought to be totally unusable, to generate local pmf to do useful work. This essentially provides a high innovative method to renew the totally “dead” latent heat energy in ambient temperature environment that according to the Second Law of Thermodynamics would be completely unusable. That is, the “dead” latent heat energy can now be reborn to create new Gibbs free energy in the form of local pmf in accordance with the invention. Therefore, it fundamentally represents a special energy-recycle and energy-renew technology.

Furthermore, it is the effective localized protons concentrations and their associated local pmf (Gibbs free energy) that fundamentally also enables the protonic industrial applications of treating substrate materials including the protonation of certain synthetic polymer films and proton-etching of substrate metal plates. Therefore, the useful work that can be done with local pmf (Gibbs free energy) includes the local pmf-driven protonation of certain protonatable synthetic polymer films and the proton-driven oxidation of certain substrate metal atoms for the industrial applications, in addition to the well-known pmf utilization for driving synthesis of ATP useful not only in living organisms but also in certain industrial biochemical engineering applications.

Therefore, exemplary embodiments provide a series of comprehensive methods for creating effective localized excess protons concentrations with a special excess proton production and utilization system including the use of an open-circuit water electrolysis process with a pair of anode and cathode electrodes in a special liquid membrane chamber system forming and using excess protons associated with an excess protons-membrane-anions capacitor-like system to enable a series of special energy recycle-related technology process functions with utilization of environmental heat energy for various special industrial applications including: a) utilization of environmental heat molecular thermal motion energy for energy recycling and renewing of the fully dissipated waste heat energy in ambient temperature environment, which conventionally is thought to be totally unusable, to generate local protonic motive force equivalent to Gibbs free energy to do useful work; b) treatment comprising protonation and proton-etching of a substrate material plate/film by forming and utilizing excess protons associated with an excess protons-membrane-hydroxyl anions capacitor-like system; and c) production and conversion of local pmf to the other ion motive force (equivalent to Gibbs free energy) for a series of other cation species for utilization of environmental heat molecular thermal motion energy and other industrial applications selected from the group consisting of Na⁺, K⁺, Li⁺, Rb⁺, Cs⁺, Co⁺⁺, Ni⁺⁺, Zn⁺⁺, Cu⁺⁺, Fe⁺⁺, Mn⁺⁺, Ca⁺⁺, Mg⁺⁺, and combinations thereof.

The present invention further discloses a method to make a water-based protonic wire comprising a proton-conductive water line filled into a protonic-insulating tube and/or a channel that may be employed in conjunction with the industrial and/or biomedical applications associated with the features of excess protons described above. According to one of the various embodiments, it is a preferred practice to use degassed liquid water to fill the tube and/or channel without forming any gas bubbles in the water line within the tube or channel. The tube and/or channel wall for a water-based protonic wire is made preferably from certain protonic-insulator materials that are impermeable to both water and protons. Depending on a given specific application and operating conditions, the cross section size or diameter of a water-based protonic wire within a tube and/or channel is selected from the group consisting of 1 nm, 2 nm, 3 nm, 4 nm, 5 nm, 6 nm, 8 nm, 10 nm, 15 nm, 20 nm, 30 nm, 40 nm, 50 nm, 100 nm, 150 nm, 200 nm, 300 nm, 400 nm, 500 nm, 600 nm, 800 nm, 1000 nm, 1500 nm, 2000 nm, 2500 nm, 3000 nm, 4000 nm, 5000 nm, 6000 nm, 8000 nm, 9000 nm, 10 μm, 11 μm, 12 μm, 13 μm, 15 μm, 16 μm, 18 μm, 20 μm, 25 μm, 30 μm, 50 μm, 100 μm, 150 μm, 200 μm, 300 μm, 400 μm, 500 μm, 600 μm, 800 μm, 1000 μm, 1500 μm, 2000 μm, 2500 μm, 3000 μm, 3500 μm, 4000 μm, 5000 μm, 6000 μm, 8000 μm, 10 mm, 11 mm, 12 mm, 13 mm 14 mm, 15 mm, 16 mm, 18 mm, 20 mm, 30 mm, 50 mm, 100 mm and/or within a range bounded by any two of these values in accordance with one of the various embodiments of the present invention. Accordingly, the length of a proton-conductive water wire is selected from the group consisting of 4 nm, 5 nm, 6 nm, 8 nm, 10 nm, 15 nm, 20 nm, 30 nm, 40 nm, 50 nm, 100 nm, 150 nm, 200 nm, 300 nm, 400 nm, 500 nm, 600 nm, 800 nm, 1000 nm, 1500 nm, 2000 nm, 2500 nm, 3000 nm, 4000 nm, 5000 nm, 6000 nm, 8000 nm, 9000 nm, 10 μm, 11 μm, 12 μm, 13 μm, 15 μm, 16 μm, 18 μm, 20 μm, 25 μm, 30 μm, 50 μm, 100 μm, 150 μm, 200 μm, 300 μm, 400 μm, 500 μm, 600 μm, 800 μm, 1000 μm, 1500 μm, 2000 μm, 2500 μm, 3000 μm, 3500 μm, 4000 μm, 5000 μm, 6000 μm, 8000 μm, 10 mm, 11 mm, 12 mm, 13 mm 14 mm, 15 mm, 16 mm, 18 mm, 20 mm, 30 mm, 50 mm, 100 mm, 200 mm, 300 mm 500 mm, 600 mm, 800 mm, 1000 mm, 1500 mm, 2000 mm, 3000 mm, 4000 mm, 5000 mm, 6000 mm, 8000 mm, 10 m, 15 m, 20 m, 30 m, 40 m, 50 m, 100 m, 200 m, 300 m, 500 m, 1000 m, and/or within a range bounded by any two of these values in consideration of given specific applications and operating conditions.

According to one of the various embodiments, the tube and/or channel wall for a proton-conductive water wire can be made from varieties of protonic-insulating materials that are selected from the group consisting of lipid bilayer, protonic-insulating membrane, myelin (a layer of a fatty insulating substance), myelin sheath, myelinated axons, certain polypeptide proton channels, silicon tubing material, plastic tubing materials, Teflon material, carbon fibers composite materials, vinyl ester, epoxy, polyester resin, thermoplastic materials, graphene, graphite, cellulose nanofiber/epoxy resin nanocomposites, protonic insulating plastics, protonic insulating ceramics, protonic insulating glass, fiberglass-reinforced plastic materials, borosilicate glass, Pyrex glass, fiberglass, sol-gel, silicone rubber, quartz mineral, diamond material, glass-ceramic, transparent ceramics, clear plastics, such as Acrylic (polymethyl methacrylate), Butyrate (cellulose acetate butyrate), Lexan (polycarbonate), and PETG (glycol modified polyethylene terephthalate), polypropylene, polyethylene (or polyethene) and polyethylene HD, protonic-insulating paint, colorless glass, clear transparent plastics containing certain anti-reflection materials, clear glass containing certain anti-reflection materials, stainless steels, metal alloys, and combinations thereof depending on a given specific application and operating conditions.

According to one of the various embodiments, a water-based protonic wire may be employed with a source of excess protons to deliver excess protons for certain industrial applications including pointed protonation and/or lithographic proton etching. Conceivably, a protonic scanner and/or writing tool may be constructed with the use of a protonic wire in combination with a source of excess protons such as those illustrated in FIGS. 1-5.

According to one of the various embodiments, the water-based protonic wires may be used in building protonic circuits that may have significant practical implications in biomedical science and technology. How does the human memory process really work? Currently, there is no definitive answer to this important scientific question. The computer memory operating process is based on electronic circuits. There are no such electronic circuits in the biological systems. Based on the fundamental understanding of water as a protonic conductor associated with the invention, the human memory process may likely operate through a type of water-based protonic circuits in addition to the known ion channels/transporters and neurotransmitters. For example, the propagation of action potential along an axon or nerve fiber (which is a long, slender projection of a nerve cell, or neuron that typically conducts electrical impulses known as action potentials) is likely by protonic conduction through the liquid water along the cell including the elongated neuron cell such as a myelinated axon as a protonic wire. The function of the axon is known to transmit information to different neurons, muscles, and glands. In this case, the cell membrane and myelin may act as a thin protonic insulator barrier around the cytosol liquid that can act as a protonic conductor. As the protons driven by action potential from one end to the other end of a neuron cell such as a myelinated axon through the cytosol (axoplasm), it may induce the formation of a transit membrane potential across the membrane at certain exposed membrane region (such as the unmyelinated region of an axon at the nodes of Ranvier, also known as myelin-sheath gaps, along a myelinated axon where the axolemma is exposed to the extracellular space) or at the other end of the cell such as an axon terminal and/or synaptic terminal, which in turn may induce protonic conduction and/or other cellular activities in the next cell and so on. This somewhat similar to the activity of induced protons propagating the membrane potential from the induction chamber 310 at the right side to the induction chamber 314 at the left side as illustrated in FIG. 3. Likewise, an action membrane potential change can propagate through protonic conduction in the reverse direction such as from the induction chamber 314 at the left side to the induction chamber 310 at the right side as well.

Since the propagation of action potential through protonic conduction can be much faster than that of a diffusion-based system, it may help better explain the propagation of action potential signals in the heart pace-making tissue that cannot be explained by a diffusion-based slow mechanism. This again indicates that our human body operates likely with a type of water-based protonic circuits. The water-based protonic wires and artificial protonic circuits may be employed in biomedical science and technology. For example, the micro/nanometer-scale water-based protonic wires and artificial protonic circuits may be used to interact with certain human and/or animal tissue cells such as neurons and axons for certain biomedical diagnosis and/or surgery treatments.

According to one of the various embodiments, the water-based protonic wires and protonic circuits may be employed in combination of localized excess protons using local protonic motive force (equivalent to Gibbs free energy) from isothermal utilization of environmental heat energy to do useful work for certain industrial processes and/or for certain biomedical science and technology applications.

### EXAMPLES

The following examples to illustrate embodiments of how the compositions and methods described herein are made and evaluated, and are intended to be purely exemplary and are not intended to limit the scope of the invention.

### Example 1: Localized Excess Protons Demonstrated with a Proton-Sensing Film

The excess proton production and utilization system 100 (as illustrated in FIG. 1) has recently been experimentally demonstrated using an “open-circuit” water electrolysis process and resulted in the formation of an “excess protons-substrate-hydroxyl anions” capacitor-like system. During the open-circuit electrolysis of pure water, excess protons were produced in the anode chamber while excess hydroxyl anions were generated in the cathode chamber.

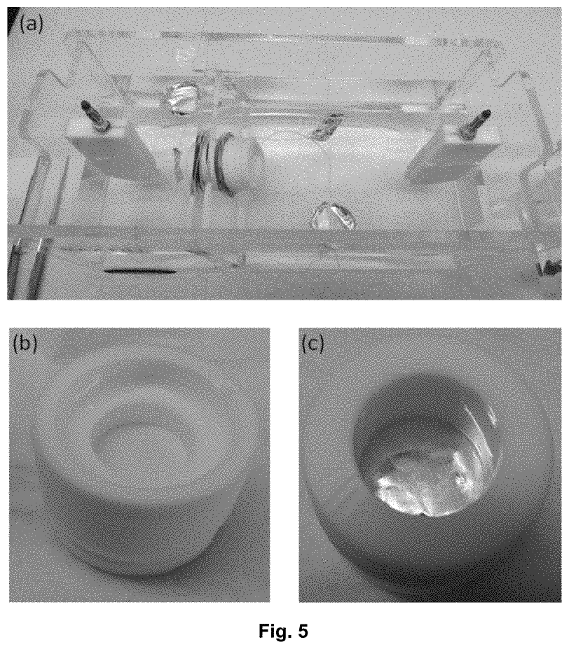

It is known that aluminum surface can begin to be corroded by protons when the effective proton concentration is above 0.1 mM (equivalent to a pH value of below 4) (Pourbaix 1974 Corrosion Science, 14(1): 25-82). This property was therefore employed as a proton-sensing mechanism in combination with the bulk phase pH electrode measurement to determine the distribution of excess protons in the water-membrane-water system (FIG. 1). In the first set of experiments (performed in triplicate), small pieces of aluminum film were employed as a protonic sensor at a number of locations in both of the water chambers to serve as an indicator for the excess protons. As illustrated in FIGS. 1 and 5, a Teflon membrane (Tf) was sandwiched in between two pieces of aluminum film (Al), forming a proton-sensing Al-Tf-Al membrane system that separate the two water bodies: the cathode water body on the left and the anode water body on the right.

The result of the “cathode water Al-Tf-Al water anode” experiment showed that only the proton-sensing Al film placed at the P_{I} site facing the anode liquid showed proton-associated corrosion (see the dark brownish grey on the exposed part of the proton-sensing film in FIG. 6). The proton-sensing film placed in the bulk liquid phase (P_{B}) of the anode chamber (or floated on the top surface (P_{S}) of the anode water body) showed no proton-associated corrosion activity since the proton-sensing film placed in the bulk liquid phase (P_{B}) remained pristine during the entire experiment (FIG. 5a). This is a significant observation since it indicates that excess protons are indeed localized primarily along the water-membrane interface at the P_{I} site, but not in the bulk liquid phase (P_{B}). Also as expected, all pieces of proton-sensing film placed at the N_{I}, N_{B}, and N_{S} sites of the cathode liquid showed no proton-associated corrosion activity as well.

According to the Mitchellian proton delocalized view, the excess protons in a water body would behave like a solute such as a sugar molecule which can stay anywhere in the liquid including its bulk liquid phase. Certain commonly heard arguments in favor of the Mitchellian proton delocalized view even as of today seem still believe that the excess protons would behave like solutes that could delocalize into the bulk liquid phase somehow by “proton solvation” or “electro diffusion”. If that delocalized view is true, it would predict that all the proton-sensing films in the anode water chamber including the one placed in the bulk liquid (P_{B}) should be able to detect the excess protons. The observation that the proton sensor placed into the anode chamber bulk water phase (P_{B}) could not detect any excess protons while the proton sensor placed at the P_{I} site showed dramatic proton-associated aluminum corrosion activity clearly rejects the Mitchellian proton delocalized view. The result clearly demonstrated the formation of localized excess protons at the water-substrate (proton-sensing Al film) interface as outlined in the invention.

### Example 2: Characterization of Excess Protons with Bulk-Phase pH Measurements

During a 10-hour experiment with 200V-driven water electrolysis, it was noticed, as expected, that the formation of small gas bubbles at both the anode and cathode platinum electrodes. This observation is consistent with the well-known water electrolysis process in which water is electrolytically oxidized to molecular oxygen (gas) producing excess protons in the anode water compartment while protons are reduced to molecular hydrogen (gas) leaving excess hydroxyl anions in the cathode water compartment. If the Mitchellian proton delocalized view is true, it would predict that the production of excess protons in the anode water compartment would result in a lower pH value for the bulk water body while the generation of excess hydroxyl anions in the cathode water body would result in a higher pH in its bulk water body. That is, if the proton delocalized view is true, it would predict a significant bulk-phase pH difference (ΔpH) between the anode and the cathode water bodies. The experimental result with the bulk-phase pH measurements demonstrated that the Mitchellian proton delocalized view is not true. As shown in Table 3, after the 10-hour experiment with the water Al-Tf-Al (membrane) water system, the measured pH value in the anode bulk water body (5.76±0.09) remained essentially the same as that of the cathode bulk water phase (5.78±0.14). These bulk water phase pH values averaged from 3 replication experiments (each replication experiment with at least 6 readings of pH measurement in each chamber water, n=3×6=18) were statistically also the same as those (5.78±0.04 and 5.76±0.02) in the control experiments in absence of the water electrolysis process. This is a significant experimental observation since it confirmed that the excess protons indeed do not stay in the bulk water phase and thus cannot be measured by a pH electrode in the bulk liquid phase.

TABLE 3
Averaged pH values measured in bulk water phase before and after 10 hours
experiment with cathode water membrane water anode systems.

| Experiments | | pH of Cathode Water | pH of Anode Water |
|---|---|---|---|
| With (Al-Tf-Al) | Before | 6.89 ± 0.03 | 6.89 ± 0.03 |
| 200 V | After | 5.78 ± 0.14 | 5.76 ± 0.09 |
| With (Tf-Al-Tf) | Before | 6.71 ± 0.10 | 6.71 ± 0.10 |
| 200 V | After | 5.81 ± 0.04 | 5.76 ± 0.03 |
| With (Al-Tf-Al) | Before | 6.89 ± 0.03 | 6.89 ± 0.03 |
| control (0 V) | After | 5.68 ± 0.06 | 5.78 ± 0.02 |
| With (Al-Tf-Al) | Before | 6.71 ± 0.10 | 6.71 ± 0.10 |
| control (0 V) | After | 5.76 ± 0.02 | 5.78 ± 0.04 |

This observation can also explain why in certain bioenergetic system such as thylakoids where ATP synthesis through photophosphorylation sometimes can occur without measurable ΔpH across the thylakoid membrane between the two bulk aqueous phases (Vinkler, Avron, Boyer 1978 FEBS Letters 96(1): 129-134). As shown in the experimental study, although the bulk-phase pH difference (ΔpH) between the anode chamber water and the cathode chamber water is zero, the excess protons were localized at the water-membrane interface as demonstrated by the dramatic proton activity on the proton-sensing film placed at the P_{I} site (FIG. 6). This indicated that the concentration of localized excess protons was much higher than 0.1 mM (equivalent to a local pH value of well below 4).

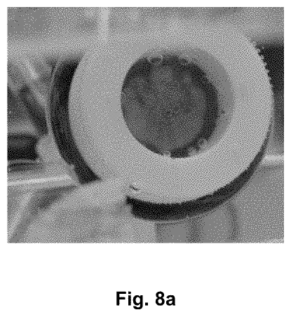

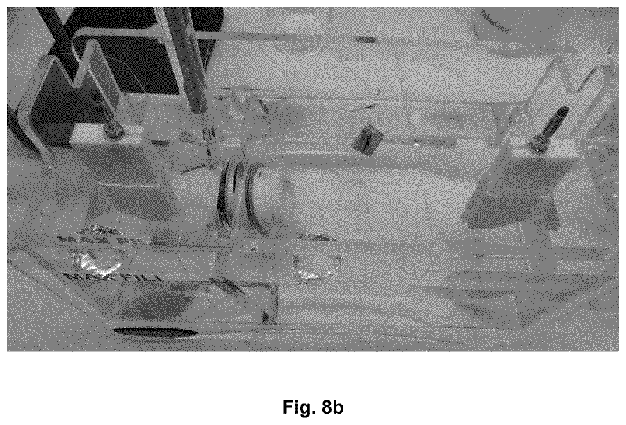

Furthermore, the measured pH value of 5.76±0.09 in the anode bulk water phase was also consistent with the observation that the piece of proton-sensing film placed in the anode bulk water phase (P_{B}) showed no sign of proton-associated corrosion (oxidation by the excess protons) activity (FIG. 6 P_{B} and FIG. 8b) while the proton-sensing film placed at P_{I} site had dramatic proton-associated corrosion (FIG. 6P₁). This indicated that the generated excess protons are indeed localized primarily at the water-membrane interface at the P_{I} site resulting in a proton surface density that is high enough (equivalent to a pH value well below 4) to cause the aluminum corrosion there.

The pH measurements also showed that the freshly deionized water had an average pH value of 6.89±0.03 before being used in the experiments (Table 3). Since the experiments were conducted in the laboratory room air, the gradual dissolution of atmospheric CO₂ into the deionized water during a 10-hour experiment period resulted in water pH change from 6.89±0.03 to 5.68±0.06, which was observed in the control experiment with the same “cathode water Al-Tf-Al water anode” setup except without turning on the electrolysis voltage (0 V). Therefore, this bulk water pH change had little to do with the 200V-driven water electrolysis process. The same magnitude of bulk water pH change before and after the experiment was observed for the deionized water in both the anode and cathode chambers, which also supports the understanding that this bulk water pH change from the beginning to the end of the experiment was due to the gradual dissolution of atmospheric CO₂ into the deionized water during the 10-hour experiment period. There was no difference between the bulk-phase pH of anode chamber water (pH 5.76±0.09) and that of the cathode chamber water (5.78±0.14) at the end of the experiment. This result also points to the same underline understanding that the excess protons do not behave like typical solute molecules. Excess protons do not stay in the water bulk phase; they localize at the water-membrane interface at the P_{I} site so that they cannot be detected by the bulk-phase pH measurement.

A further set of experiments with the setup of “cathode water Tf-Al-Tf water anode” was also conducted in triplicate. In this set of experiments, we chose to use the Tf-Al-Tf membrane system instead of the Al-Tf-Al membrane system. Since the Teflon membrane is chemically inert to protons, the use of the Tf-Al-Tf membrane system eliminated the consumption of excess protons by the aluminum corrosion process at the P_{I} site that was demonstrated above. In this set of the experiments, no bulk-phase pH difference (ΔpH) between the anode and cathode water bodies was observed as well. As shown in Table 3, after run for 10 hours at 200V with the “cathode water Tf-Al-Tf water anode” system, the measured pH value in the anode bulk water phase (5.76±0.03) was essentially the same as that of the cathode bulk water phase (5.81±0.04). This experimental observation again indicated that the excess protons do not stay in the bulk water phase and thus cannot be measured by the bulk liquid phase pH measurement. Since liquid water is an effective proton conductor as discussed above, the excess protons produced in the anode water compartment electrostatically localize to the water-membrane interface at the P_{I} site, where they also attract the excess hydroxyl anions of the cathode water body at the other side of the Tf-Al-Tf membrane, forming an “excess hydroxyl anions Tf-Al-Tf excess protons” capacitor-like structure as illustrated in FIG. 1.

### Example 3: Production of Excess Protons Assessed with Water Electrolysis Electric Current Measurements

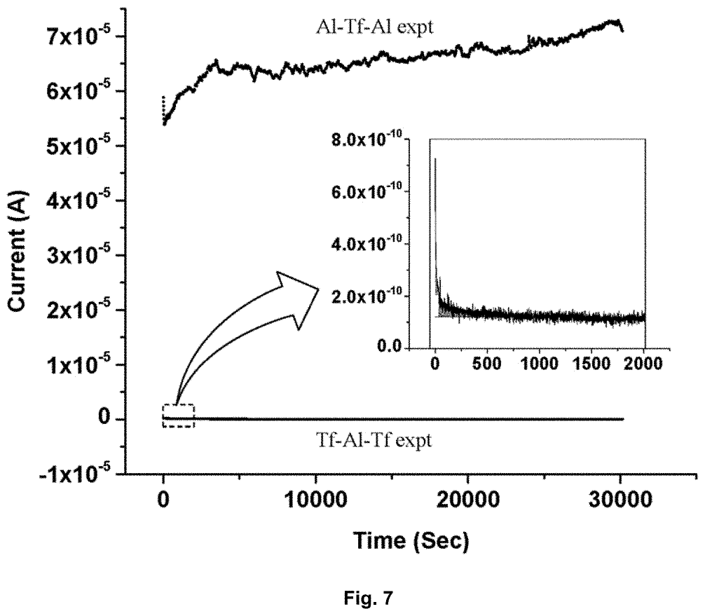

The proton-charging-up process in this “excess hydroxyl anions Tf-Al-Tf excess protons” capacitor system was monitored by measuring the electric current of the 200V-driven water electrolysis process as a function of time during the entire 10-hour experimental run. The data in the inset of FIG. 7 showed that the electric current of the water electrolysis process decreased with time as expected. That is, when the excess protons were generated in the anode water compartment (while the excess hydroxyl anions were generated in the cathode water compartment), this “excess hydroxyl anions Tf-Al-Tf excess protons” capacitor is being charged up by localization of the excess protons at the P_{I} site and the excess hydroxyl anions at N_{I} site (FIG. 7). According to the analysis, this process reached thermodynamic equilibrium after 1500 seconds (shown in the inset of FIG. 7) under this experimental condition where the curve of the water electrolysis current quickly became flat indicating the completion of the water electrolysis-coupled protonic-charging-up process.

By calculating the area under the water-electrolysis current curve above the flat baseline as shown in the inset of FIG. 7, the amount of excess protons loaded onto the “excess hydroxyl anions Tf-Al-Tf excess protons” capacitor was estimated to be about 2.98×10⁻¹³ moles (Table 4). The area of the Teflon membrane surface exposed to the anode water at the P_{I} site was measured to be 2.55 cm². If that amount of excess protons were loaded at the P_{I} site onto the Teflon membrane surface exposed to the anode water, the maximal localized excess proton density per unit area was estimated to be 1.19 nanomoles H⁺/m². Although the exact thickness of the localized excess proton layer at the P_{I} site is yet to be determined, studies indicated that the effective thickness for this type of the electrostatically localized excess proton layer may be about 1±0.5 nm. If that is the case, then the localized excess proton density of 1.19 nanomoles H⁺/m² would translate to a localized excess proton concentration of 1.19 mM H⁺ (equivalent to a localized pH value of 2.92) at the P_{I} site, which can explain why they can be detected by the protonic-sensing Al film there.

TABLE 4
Calculation of localized proton density per unit area in the
“cathode water Tf-Al-Tf water anode” experiment.

| | Area under the | Moles of | Localized | |
|---|---|---|---|---|
| | current vs. | excess | proton density | pH at P_{I} |
| | time curve | protons | per unit area | of the |
| | (Coulombs) | H⁺ (mol) | (mole H⁺/m²) | Tf-Al-Tf |
| Trial 1 | 3.03 × 10⁻⁸ | 3.14 × 10⁻¹³ | 1.25 × 10⁻⁹ | 2.90 |
| Trial 2 | 2.25 × 10⁻⁸ | 2.33 × 10⁻¹³ | 9.33 × 10⁻¹⁰ | 3.03 |
| Trial 3 | 3.35 × 10⁻⁸ | 3.47 × 10⁻¹³ | 1.38 × 10⁻⁹ | 2.85 |
| Average | 2.88 × 10⁻⁸ | 2.98 × 10⁻¹³ | 1.19 × 10⁻⁹ | 2.92 ± 0.09 |

The water electrolysis current in the “cathode water Al-Tf-Al water anode” experiment was also monitored. As shown in FIG. 7, after 5000 seconds, the water electrolysis electric current at the steady state of this experiment reached around 6.5×10⁻⁵ A, which was much bigger than that (below 1×10⁻¹⁰ A) of the “cathode water Tf-Al-Tf water anode” experiment. This large water electrolysis electric current can be attributed to the consumption of excess protons by the protonic-sensing Al film at the P_{I} site. As the protonic-sensing film at the P_{I} site consumes the excess protons, more excess protons can then be produced at the anode electrode, resulting in a significant water-electrolysis electric current. The high concentration of the electrostatically localized excess protons at the P_{I} site thermodynamically drives the aluminum corrosion reaction in which aluminum atoms are oxidized by protons resulting in evolution of molecular hydrogen gas. During the experiment, we indeed noticed the formation of gas bubbles on the aluminum membrane surface at the P_{I} site (FIG. 8a), which is consistent with the understanding of the localized excess protons-driven aluminum corrosion process [Eq. 5] mentioned above.

By calculating the area under the water-electrolysis current curve from the “cathode water Al-Tf-Al water anode” experiment and subtracting that of the “cathode water Tf-Al-Tf water anode” experiment, the amount of excess protons that were generated by the anode and consumed by the protonic-sensing film at the P_{I} site was able to be calculated. As shown in Table 4, during the 10-hr “cathode water Al-Tf-Al water anode” experiment, a total of 2.11×10⁻⁵ moles of excess protons were generated by the anode platinum electrode. These excess protons were apparently translocated to the protonic sensing Al film surface at the P_{I} site and consumed there by the corrosion reaction which gives the dark brownish grey on the exposed part of the protonic sensing Al film as shown in FIGS. 6 (P_{I}) and 8a. The amount of protons consumed per unit area was calculated to be 8.29×10⁻⁶ moles per cm².

### Example 4: Experimental Demonstration with the Three-Chamber System

Recently, in the Lee laboratory at Δψ Dominion University (ODU), excess protons and excess hydroxyl anions were generated utilizing a three-chamber system (comprising a cathode chamber, a Teflon sample (induction) chamber, and an anode chamber) through application of a special “open-circuit” water-electrolysis process, which is similar to the 200 system (FIG. 2). A Teflon sample chamber was sealed at both ends by two pieces of proton-sensing films placed along with an impermeable (Teflon) membrane in between. This Teflon chamber was filled with liquid water, and was then tightly fit through a specific hole in the wall that separates the anode and cathode chambers so that one of the sample chamber ends is in contact with cathode bulk liquid while the other end in contact with anode bulk liquid.

Based on the experimental observations, when excess protons were generated in the anode water body while excess hydroxyl anions were generated in the cathode chamber water through the “open-circuit” water-electrolysis process that was carried out for 20 hours, the excess protons in the anode water were localized at the water-membrane interface along the Teflon membrane surface forming a positive (P) side. The localized protons of the P side attracted the hydroxyl anions of the middle sample chamber water to the water-membrane interface at the other side of the Teflon membrane, forming an induced negatively charged hydroxyl anions layer (N′) also shown at the INI site 212 in FIG. 2. In addition, the excess hydroxyl anions in the cathode water were localized at the water-membrane interface along the Teflon membrane surface forming a negative (N) side also shown as the excess anions layer at NI site 208 in FIG. 2. The localized hydroxyl anions of the N side attract the protons of the middle sample (induction) chamber water to the Teflon membrane's P′ side (induced protons layer at IPI site 211 in FIG. 2) facing the induction chamber water.

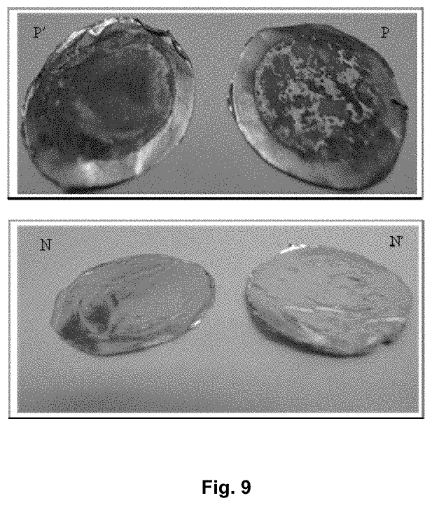

The experimental result that supports this understanding is shown in FIG. 9. The protonic-sensing Al film placed at the P or P′ side of Teflon membrane detected the localized proton activity so that its color became dark brownish grey (FIG. 9, top row); while the proton-sensing Al film placed at the N or N′ side of Teflon membrane detected no significant proton activity so that its color remains unchanged (bottom row of FIG. 9).

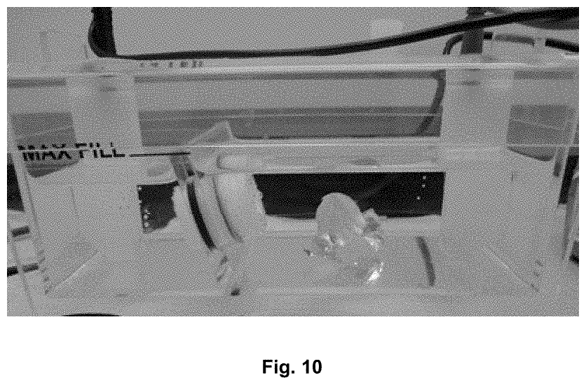

To see if the excess protons in the anode water could stay inside the anode water body, a piece of protonic-sensing Al film material was inserted into the anode chamber water body as shown in FIG. 10. The protonic-sensing film material inserted into the anode chamber water detected no significant proton activity (the pristine film color remained the same) during the entire 20 hour experiment (FIG. 10); whereas the localized protons were detected by the color change of the proton-sensing film placed at the P side surface at the right end of the Teflon sample (induction) chamber facing the anode water. These experimental results indicated: (i) The excess protons generated in the anode water did not stay inside the anode water body; and (ii) The localization pattern of the excess protons and hydroxyl anions along the two sides of the Teflon membrane is similar to that illustrated in FIGS. 1 and 2.

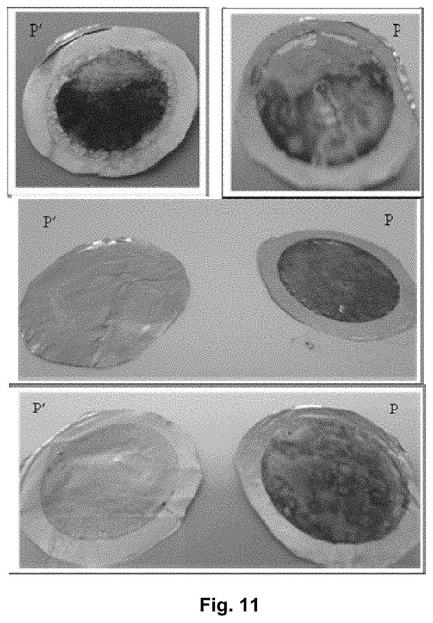

A further experiment was performed by introducing certain salt (sodium bicarbonate) into the Teflon sample chamber (FIG. 10) to test the effect of sodium cations of the salt solution on the localized protons at the induced P′ side in the sample chamber, in comparison with the unperturbed P side facing the anode water. The experimental results (FIG. 11) showed that the addition of 10 mM sodium bicarbonate had no significant effect on the localized protons at the P′ side facing the sodium bicarbonate solution. The use of 100 mM sodium bicarbonate (in the sample chamber) led to the reduction of localized protons at the P′ side by about 50%, which was monitored by the color change of the protonic-sensing film at the P′ side, in comparison with that of the protonic-sensing film placed at the P side (FIG. 11, bottom row). It required the use of 400 mM or higher concentration of sodium bicarbonate solution in Teflon sample (induction) chamber to remove the localized protons at the P′ side to a level that could not be detected by the proton-sensing film (FIG. 11, middle row).

Based on this experimental observation, the exchange equilibrium constant of sodium (Na⁺) cations with the localized protons was estimated to be less than 10⁻⁷. That is, the electrostatically localized protons at the water-surface interface is quite stable, in that it would require more than 10⁺⁷ times more Na⁺ cations than the protons in the liquid phase to delocalize the protons from the water-membrane interface at the P′ site. This gives confidence that the proton-electrostatic localization hypothesis is a correct and robust concept, which is employed in the invention.

### Example 5: Application of Localized Excess Protons for Utilizing Environmental Heat Energy to Generate “Bonus” Protonic Motive Force

In this example, 1.5 V of electrolytic voltage is applied across the anode and the cathode in a multi-chamber system similar to the one illustrated in FIG. 4 that produces excess protons and hydroxyl anions forming multiple “excess protons-membrane-hydroxyl anions” capacitor-like structures for extraction of environmental heat to generate additional protonic motive force (equivalent to useful Gibbs free energy) to do work such as driving ATP synthesis. In this multi-chamber system, there are 15 membranes that separate 16 liquid chambers. Each chamber contains the pH 7.0 liquid media as listed in Table 2. At the equilibrium with the excess-proton-producing water electrolysis process driven by the 1.5 V across the anode and the cathode, the membrane potential across each of the 15 membranes is 100 mV, which, if based on the delocalized proton view of Peter Mitchell's Chemiosmotic Theory, would translate to a classic pmf of only 100 mV that would not be sufficient to drive ATP synthesis to support cell growth. On the other hand, according to the data of Table 2 of the invention, with a membrane potential of 100 mV and liquid media pH 7.0, each membrane has a total pmf of 398 mV (298 mV of it is from the local pmf) that is sufficient to drive proton users such as ATP synthase for synthesis of ATP from ADP and Pi. The total pmf of the 15 membranes is 5.97 V, of which 4.47 V is from the local pmf that is extracted from the environmental heat with the localized protons at the membrane surfaces and the remainder 1.5 V is the total membrane potential. That is, the use of a 1.5 V water electrolysis process through this special system generates a total pmf of 5.97 V. In this example, 74.9% of the total pmf (5.97 V) is generated from the environmental heat (thermal motion energy) by the activity of localized protons in accordance with one of the various embodiments of the present invention.

### Example 6: Application of Localized Excess Protons for Utilizing Environmental Heat Energy to Generate More “Bonus” Protonic Motive Force

In this example, 1.5 V of electrolytic voltage is applied across the anode and the cathode in a multi-chamber system similar to the one illustrated in FIG. 4 that produces excess protons and hydroxyl anions forming multiple “excess protons-membrane-hydroxyl anions” capacitor-like structures for extraction of environmental heat to generate additional protonic motive force (equivalent to Gibbs free energy) to do useful work such as driving ATP synthesis. In this multi-chamber system, there are 30 membranes that separate 31 liquid chambers: each chamber contains the pH 7.0 liquid media as listed in Table 2. At the equilibrium with the excess-proton-producing water electrolysis process driven by the 1.5 V across the anode and the cathode, the membrane potential across each of the 30 membranes is 50 mV, which, if based on Mitchell's delocalized proton view, would translate to a classic pmf of only 50 mV that would not be sufficient to drive ATP synthesis to support cell growth. On the other hand, according to the data of Table 2 of the present invention, with a membrane potential of 50 mV and liquid media pH 7.0, each membrane has a total pmf of 330 mV (280 mV of it is from the local pmf) that is sufficient to drive proton users such as ATP synthase for synthesis of ATP from ADP and Pi. The total pmf of the 30 membranes is 9.9 V, of which 8.4 V is from the local pmf that is extracted from the environmental heat energy with localized protons at the membrane surfaces, and the remainder 1.5 V is the total membrane potential. That is, a 1.5 V input through this special system generates a total pmf of 9.9 V. In this case, 84.8% of the total pmf (9.9 V) is generated from environmental heat (thermal motion energy) by the activity of localized protons. Note, the total pmf (9.9 V) generated in this example is significantly higher than that in Example 5 that uses 15 membranes (each with 100 mV of membrane potential). This result demonstrates that the use of more (30) membranes (each with 50 mV of membrane potential) can indeed generate more local pmf (8.4 V) from environmental heat than that (4.47 V) of Example 5 using 15 membranes (each with 100 mV of membrane potential) even though the same 1.5 V of electrolytic voltage is used in both Examples 5 and 6, which is consistent with the predicted feature from the present invention.

### Example 7: Application of Localized Excess Protons for Utilizing Environmental Heat Energy to Generate Much More “Bonus” Protonic Motive Force

In this example, 1.5 V of electrolytic voltage is applied across the anode and the cathode in a multi-chamber system similar to the one illustrated in FIG. 4. In this multi-chamber system, there are 60 membranes that separate 61 liquid chambers: each chamber contains the pH 7.0 liquid media as listed in Table 2. At the equilibrium with the excess-proton-producing water electrolysis process driven by the 1.5 V across the anode and the cathode, the membrane potential across each of the 60 membranes is 25 mV, which, if based on Mitchell's delocalized proton view, would translate to a classic pmf of only 25 mV that would not be sufficient to drive ATP synthesis to support cell growth. On the other hand, according to the data of Table 2 of the present invention, with a membrane potential of 25 mV and liquid media pH 7.0, each membrane has a total pmf of 288 mV (263 mV of it is from the local pmf) that is sufficient to drive proton users such as ATP synthase for synthesis of ATP from ADP and Pi. The total pmf of the 60 membranes is 17.28 V, of which 15.78 V is from the local pmf that is extracted from environmental heat energy with localized protons and the remainder 1.5 V is the total membrane potential. That is, the use of a 1.5 V water electrolysis energy input generates a total pmf of 17.28 V. In this case, 91.3% of the total pmf (17.28 V) is generated from environmental heat (thermal molecular motion energy) extracted by the activity of localized protons. Note, this total pmf (17.28 V) is much higher than that in Example 6 that uses 30 membranes (each with 50 mV of membrane potential). This again demonstrates that the application of more membranes (60, each with 25 mV of membrane potential here) in accordance with one of the various embodiments can indeed extract much more environmental heat energy by localized protons to generate much more local pmf (15.78 V) than that of Examples 5 and 6 with the same 1.5 V electrolysis voltage input.

### Example 8: Application of Localized Excess Protons for Utilizing Environmental Heat Energy to Generate Additional Proton Motive Force—Biological Implications

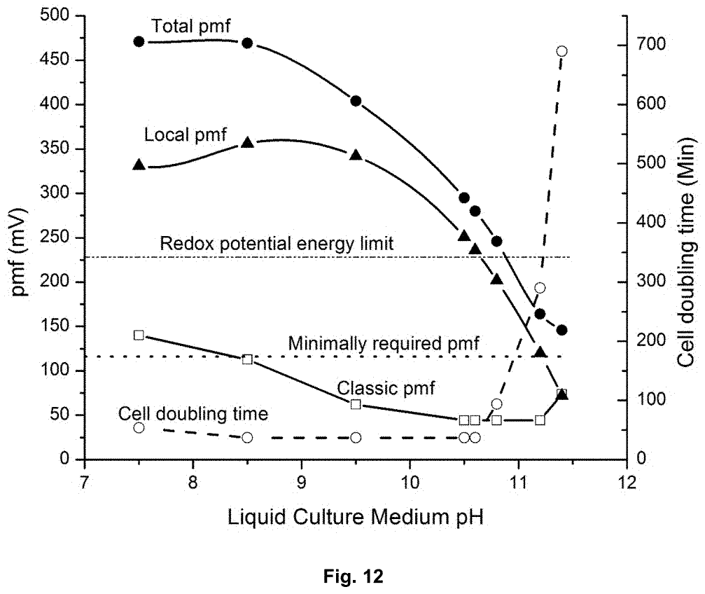

Table 5 shows pmf values calculated from Eqs. 6-8 based on the well-established experimental data of Bacillus pseuodofirmus OF4 (alkalophilic bacteria) under its culture medium pH, cytoplasmic pH, and transmembrane potential conditions. The calculated pmf as a function of the culture medium pH is displayed in FIG. 12, in comparison to the “classic” pmf contribution. From these calculated results, it is apparent that the local pmf contributed by the surface localized protons dominates the overall strength of the total protonic motive force.

As shown in FIG. 12, the total pmf values including the local pmf contribution from the localized protons are well above the minimally required value of at least 116 mV, while the classical pmf is significantly below this minimum requirement for at all liquid culture pH values above 8.5. The minimum pmf value is what is needed to overcome the known phosphorylation potential (−497 mV) for ATP synthesis through the ATP synthase with a proton-to-ATP ratio of 13/3 (497 mV/4.33=116 mV). The proton-to-ATP ratio of 13/3 used in this calculation for the minimally required value of 116 mV is consistent with the known structure of Bacillus pseuodofirmus F₀F₁-ATP synthase, which has 3 catalytic sites for ATP synthesis driven by a flow of 13 protons per revolution through the 13 c-subunits in its nanometer-scale molecular turbine ring.

The calculated total pmf values as listed in Table 5 are in a range from 468 mV to 161 mV, which are all above the minimally required value of 116 mV. Especially, when the culture medium pH in a range from 7.5 up to 10.8, the calculated total pmf value are in a range from 468 mV to 260 mV, which is well above the minimally required 116 mV. This result can explain why the Bacillus pseuodofirmus OF4 culture can keep such an excellent growth rate (doubling times less than 100 min) in this culture pH range from 7.5 to 10.8. Furthermore, the decrease in pmf when the liquid culture pH is raised beyond 10.8, due to decreased contribution from the localized protons, matches well with the dramatic increase in the measured growth doubling times (decreased growth rate).

Theoretically, when the total pmf is reduced to around 116 mV, the bioenergetic system would reach equilibrium and the molecular turbine of F₀F₁-ATP synthase would stop running and the culture growth could completely stop. When the total pmf values is reduced to a value somewhat closer the minimally required value of 116 mV, such as 179 mV and 161 mV as calculated at the culture pH 11.2 and 11.4, the growth rate would grammatically decrease. This understanding, for the first time, provides an excellent bioenergetics explanation in correlating with the dramatic reduction of growth rate observed at culture pH 11.2 and 11.4 (Table 5 and FIG. 12).

The successful elucidation of the decades-longstanding energetic conundrum of alkalophilic bacteria Bacillus pseuodofirmus OF4 as to how they are able to synthesize ATP as demonstrated again in this Example 8, also indicated that the local pmf values calculated through Eqs. 3 and 6-8 using the parameters reported above are indeed about right.

TABLE 5
Bacillus pseuodofirmus OF4 measured properties (pH_{pB}, pH_{nB}, Δψ) and calculated
quantities using Eqs. 6-8. The cation concentrations and proton exchange equilibrium constants
are from Table 1 and the temperature T = 298K. The “local” pmf is the last term in Eq. 8 due to
the localized protons, while the first two terms of Eq. 8 give the “classic” pmf.

| | | | | Exchange | | Local | Classic | Total |
|---|---|---|---|---|---|---|---|---|
| | | Δψ | [H_{L}⁺]⁰ | reduction | [H_{L}⁺] | pmf | pmf | pmf |
| pH_{pB} | pH_{nB} | (mV) | (molar) | factor | (molar) | (mV) | (mV) | (mV) |
| 7.5 | 7.5 | 140 | 1.92 × 10⁻² | 1.71 | 1.12 × 10⁻² | 328 | 140 | 468 |
| 8.5 | 7.7 | 160 | 2.19 × 10⁻² | 8.60 | 2.54 × 10⁻³ | 349 | 113 | 462 |
| 9.5 | 7.5 | 180 | 2.46 × 10⁻² | 133 | 1.85 × 10⁻⁴ | 341 | 62 | 403 |
| 10.5 | 8.2 | 180 | 2.46 × 10⁻² | 2.77 × 10⁴ | 8.88 × 10⁻⁷ | 263 | 44 | 307 |
| 10.6 | 8.3 | 180 | 2.46 × 10⁻² | 6.06 × 10⁴ | 4.06 × 10⁻⁷ | 249 | 44 | 293 |
| 10.8 | 8.5 | 180 | 2.46 × 10⁻² | 3.39 × 10⁵ | 7.27 × 10⁻⁸ | 216 | 44 | 260 |
| 11.2 | 8.9 | 180 | 2.46 × 10⁻² | 2.01 × 10⁷ | 1.23 × 10⁻⁹ | 135 | 44 | 179 |
| 11.4 | 9.6 | 180 | 2.46 × 10⁻² | 2.13 × 10⁸ | 1.16 × 10⁻¹⁰ | 87 | 74 | 161 |

### Example 9: Application of Localized Excess Protons for Utilizing Environmental Heat to Generate Additional Protonic Motive Force Revealing a Special Biological Energy Function

As noted, the pmf values predicted by Eq. 8 for Bacillus pseuodofirmus OF4 were all larger than the minimum value required for ATP synthesis; however, the pmf values for the culture at pH 7.5, 8.5, 9.5, and 10.5 of 468 mV, 462 mV, 403 mV, and 307 mV, respectively, are all significantly larger than the maximum value of 228 mV that would be allowed by the First and the Second Laws of Thermodynamics (see FIG. 12). Since the additional “bonus” pmf is somehow from an isothermal utilization of environmental heat energy, it perfectly obeys the First Law (Conservation of Energy and Mass). The implication of the “bonus” local pmf values listed in Table 5 (and Table 2) is on the Second Law of Thermodynamics, which states the impossibility of utilizing or extracting the dissipated environmental heat energy from ambient temperature environment to do useful work.

The maximum pmf value allowed by the conventional Thermodynamics for the entire respiratory redox-driven proton-pumping system such as the one in Bacillus pseuodofirmus OF4 is only 228 mV as presented in FIG. 12 as a redox Gibbs free energy limit. This number can be calculated from the redox potential difference between the electron donor NADH to the terminal electron acceptor O₂ in this system which is known to be about 1140 mV (Nicholls and Ferguson 2013 Bioenergetics, 27-51, Academic Press) and from the number of protons that are translocated across the membrane for each pair of electrons from NADH to pass through the respiratory chain to O₂, it drives the translocation of 10 protons across the membrane from the cytoplasm to the culture medium outside the cell. That is, it couples the translocation of 5 protons per electron across the membrane. Therefore, the thermodynamically predicted maximum pmf that could be generated is about 228 mV per proton (1140 mV/5 protons) under the standard conditions (pH 7.0).

The classic Mitchellian pmf values calculated from the first two terms of Eq. 8 as listed in Table 5 and presented in FIG. 12 are far below this 228 mV limit. When the bacteria culture medium pH was around 10.5, the classic Mitchellian pmf value got as low as 44 mV which clearly could not explain the observed excellent cell growth rate. Apparently, it is the local pmf which can now be calculated through the third term of Eq. 8 from the localized proton concentration at the water-membrane interface that contributes more than 200 mV of “bonus” pmf in supporting the observed excellent cell growth rate. At culture medium pH 10.5, the local pmf is 263 mV which represents as much as 85% of the total pmf (307 mV) while the classic Mitchellian pmf (44 mV) represents only 15% of the true total pmf.

The total pmf (307 mV) is significantly higher than the conventionally predicted pmf upper limit of 228 mV for the redox-driven proton-pumping system, which is also known as the thermodynamics limit. Therefore, if the observed pmf value in the oxidative-respiratory phosphorylation system such as the one in Bacillus pseuodofirmus OF4 is truly exceeds this limit, it could indicate that something special in the biological system might not necessarily have to obey the Second Law of thermodynamics.

### Example 10: Application of Localized Excess Protons for Extraction of Environmental Heat Energy Revealing a Special Anti-Second-Law Energy Function

Regarding whether a total pmf value much higher than the thermodynamics limit of 228 mV would imply that electrostatically localized protons do not exist at the cell membrane surface, or that they are not taken into account properly by Eqs. 6-8, it is now believed that the work done by the localized protons in producing ATP is not constrained by the Second Law of Thermodynamics for the following reasons.

First, the localized protons are not entirely free to move; they are electrostatically held at the membrane surface. Consequently, their thermal (Brownian) motion will cause some to enter the opening of the ATP synthase and be used to produce ATP. Secondly, the localized protons must not be directly coupled to the redox proton pumps. If they were, they would be constrained by the Second Law and they would also disrupt the respiratory process. A natural explanation of why this does not occur is that the exit points for the translocated protons must be outside of the surface layer of the electrostatically localized protons. Furthermore, to effectively make use of the localized proton thermal motion, the proton entry point for ATP synthase must be inside the localized proton surface layer. In this scenario, the redox-driven proton pump activity interacts with the proton activity in the bulk liquid phases but not with that of the localized proton layer at the liquid-membrane interface. Only the transmembrane electric potential difference and the bulk-phase proton activity at the two sides of the membrane interact and equilibrate with the proton-pumping respiratory chain activity which is driven by 228 mV per proton and follows the Second Law. The localized proton thermal motion provides additional free energy that may be utilized by the ATP synthase.

Regarding the determination of the structures of the redox complexes in sufficient detail to confirm, or disprove, these conjectures, the structures of bacterial respiratory membrane protein complexes are not well known yet. However, they are believed to be very similar to those in mitochondria, which have been more extensively studied. Indeed, the known structures of the mitochondrial respiratory protein complexes, as determined by cryo-electron microscopy and other molecular structural studies (Dudkina et al., 2010 Biochimica Et Biophysica Acta-Bioenergetics, 1797(6-7): 664-670), fit well with the fundamental understanding and principle associated with the invention. Every one of the mitochondrial respiratory redox-driven proton-pumping protein complexes I, III and IV are indeed protruded away from the membrane surface by about 1-3 nm into the bulk liquid, while the end (protonic mouth) of the ATP synthase (complex V) is located indeed rightly at the membrane surface within the localized proton layer as predicted by one of the various embodiments in the invention.

Therefore, the electrostatically localized protons in combination with asymmetric structural features of the biological membrane especially in regarding to the positions of the proton pump outlets and the mouth of the localized proton users such as that of the ATP synthase (complex V) with respect to the localized proton layer along the p-side of the membrane may constitute this special function, which is not necessarily constrained by the Second Law of Thermodynamics. It is the electrostatic proton localization with the effect of water as a proton conductor that enables the formation of localized excess proton layer at water-membrane interface over the mouths of the pmf users including the F₀F₁-ATP synthase. The formation of a localized excess proton layer at water-membrane interface apparently results in some kind of “negative entropy effect” that bring the excess protons to the mouths of the pmf users where the protons can utilize their molecular thermal motions (environmental heat energy) possibly including their Brownian motion to push through the doors of F₀F₁-ATP synthase in driving ATP synthesis.

In order to avoid the situation of localized excess protons pushing the “wrong doors” such as the proton exit sites of the respiratory electron-transport-coupled proton pumps, the billion-year natural evolution process apparently has already solved this potential problem by protruding all the proton pump exits of the respiratory protein complexes I, III and IV a few nanometers away from the membrane surface into the bulk liquid phase while keeping the mouth of the ATP synthase (complex V) rightly at the membrane surface for the best benefit of utilizing the localized excess protons there. In this way, the localized excess protons at the water-membrane interface along the membrane surface can perfectly go through the mouth of ATP synthase (complex V) and they will not be able to touch the “doors” of the redox-driven proton-pumping respiratory protein complexes I, III and IV that are protruded into the bulk liquid phase well out of the localized excess layer as we can now start to understand.

The benefit for such an apparently Anti-Second-Law biological function is significant. The application of Eq. 9 has now, for the first time, been able to calculate the “local pmf” as listed in Table 5 and plotted in FIG. 12, which represents the amount of pmf (equivalent to Gibbs free energy) extracted isothermally by this Anti-Second-Law biological function from the dissipated ambient-temperature heat energy of the bacteria culture medium environment. The pmf (useful free energy) extracted from the environmental heat energy may represent as much as 85% of the total pmf (307 mV) for the Bacillus pseuodofirmus growing at pH 10.5, which beautifully explains the observed excellent cell growth rate that Peter Mitchell's chemiosmotic theory completely fails to explain.

From this example, it is now also clear that the creation of localized excess protons contributes to conferring this special Anti-Second-Law energy technology function that enables the utilization of dissipated environmental heat from the ambient temperature environment to generate additional protonic motive force (equivalent to Gibbs free energy) that can be employed to do useful work. Furthermore, the asymmetric features of the membrane, especially with regarding to the geometric position of proton producers with their outlets extended well into the bulk phase liquid while the mouths of proton users being rightly within the localized excess protons layer along the membrane surface, is also beneficial to effectively employing the localized excess protons to serve as the key part of the special Anti-Second-Law energy technology function. This conclusion is also consistent with the fundamental understanding and the spirit demonstrated through the invention. For example, as mentioned above, it is a preferred practice to place the proton-generating anode electrode well into the bulk phase liquid as illustrated in FIGS. 1-4 to produce more desirable results in accordance with the various embodiments of the invention.

### Example 11: Experimental Demonstration of Water-Based Protonic Wires

In this example, a number of water-based protonic wires with a series of lengths in a range from 50 to 350 cm were experimentally demonstrated by measuring their protonic DC conductivity. In the experiments, two chambers (each equipped with a platinum electrode) were each filled with 600 ml of ultrapure de-ionized water (MilliQ, Millipore Corporation, USA) at room temperature 22.5° C. The conventional electric conductivity of the ultrapure deionized water was measured with an AC conductivity meter integrated within the Millipore synergy water system and was determined to be 0.055 μS cm⁻¹ (resistivity 18.2 MΩ·cm at 22.5° C.). The two water chambers were positioned 30 cm apart and bridged by a silicon tube with an inside diameter of 3 mm that was filled with a continuous pure water column to serve as a protonic wire. A number of water-based protonic wires (in silicon tubes) with a series of tube lengths (50, 100, 150, 200, 275, 350 cm) were each tested separately. For each water tube, one of its two end openings was immersed in the anode chamber water and the other immersed in the cathode chamber water. Each experiment was performed under Direct Current (DC) by sweeping voltage across the anode and cathode platinum electrodes using digital multimeter system (Keithley instruments series 2400S-903-01 Rev E). Different voltages were applied, starting with low non water-electrolyzing potential 0.2 V, and ending with high water-electrolyzing potential 210V. In all experiments, the resulting electric current (I) and resistance (R) were measured using the same digital electrometer integrated—via GPIB cable—with KickStart (version 1.8.0) software. By using this experimental setup, the DC protonic conductivity of the water wires was successfully measured. The DC protonic conductivity of the water-based protonic wires under the experimental conditions was determined to be 1.206×10⁻⁶ S/cm, which is 22 times more than the conventional electric conductivity of water (0.055×10⁻⁶ S/cm). This experimental result demonstrated the functional property of water-based protonic wires and provided further evidence that excess protons in liquid water behave like electrons in metallic conductor, which again supports the invention.

Methods for Energy Renewal with Isothermal Electricity Production

Through the work associated with localized excess protons disclosed above, it was revealed that environmental heat, also known as latent (existing hidden) heat energy, can be isothermally utilized through electrostatically localized protons at a liquid-membrane interface to do useful work in driving the synthesis of ATP without being constrained by the second law of thermodynamics as shown in FIG. 4. This type of proton associated isothermal environmental heat utilization process apparently occurs in many proton-coupling bioenergetics systems such as the alkalophilic bacteria and the animal mitochondria. The case of the alkalophilic bacteria bioenergetics (FIG. 12) probably represents just a tip of an iceberg to the non-second-law component of the world that had not been fully recognized before. It is now quite clear that the life on Earth likely comprises a mixture of both the second-law and the anti-second-law processes that apparently have been going on naturally for billions of years. For example, some biological processes such as the metabolic process of glycolysis appear to follow the second law of thermodynamics very well; On the other hand, the membrane potential (A associated local protonic motive force as expressed in the local pmf equation (Eq. 9) disclosed above clearly represents an anti-second-law energy-renewal mechanism. This breakthrough fundamental understanding may have game-changing practical implications on new energy technology development for sustainable development on Earth. As inspired by the fundamental understanding of the proton-based energy-renewing processes described above, the present invention further discloses an electron-based energy renewal method to isothermally utilize environmental heat energy with thermal electrons for electricity generation hereinbelow.

The present invention here is directed to an energy renewal method for generating isothermal electricity with a special asymmetric function-gated isothermal electricity power generator system comprising at least one pair of a low work function thermal electron emitter and a high work function electron collector across a barrier space installed typically in a container such as a vacuum chamber or bottle with electric conductor support to enable a series of energy recycle process functions with utilization of environmental heat energy isothermally for at least one of: a) utilization of environmental heat energy for energy recycling and renewing of fully dissipated waste heat energy from the environment to generate electricity with an output voltage and electric current to do useful work; b) providing a novel refrigeration cooling function without requiring any of the conventional refrigeration mechanisms of compressor, condenser, evaporator and/or radiator by isothermally extracting environmental heat energy from inside the cold box (the heat source) while generating isothermal electricity; and c) combinations thereof.

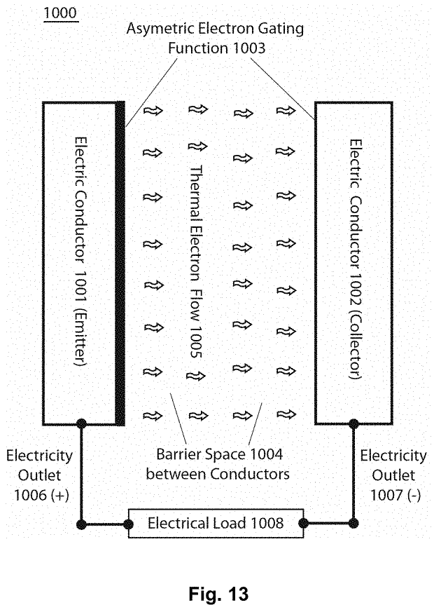

According to one of the various embodiments, this electron-based energy renewal method teaches how to isothermally extract environmental heat energy to generate electricity by teaching the making and using of an asymmetric function-gated isothermal electron-based power generator such as the asymmetric electron-gated system 1000 illustrated in FIG. 13. The system 1000 (FIG. 13) comprises an asymmetric electron-gating function 1003 across a membrane-like barrier space 1004 that separates two electric conductors 1001 and 1002 acting as a pair of a thermal electron emitter and an electron collector, two electrically conducting leads 1006 and 1007 connected with each of these electrodes 1001 and 1002 as the two power outlet terminals that may be connected with an electrical load 1008. The barrier space 1004 is preferably a special electric insulator which contains no electric conduction materials (does not conduct electrons through any molecular orbital-associated conduction bands) but allows the thermally emitted electrons to fly through ballistically across the emitter and collector.

Therefore, according to one of the various embodiments, the barrier space 1004 comprises a vacuum space that has no electric conductive materials and/or molecules with molecular orbital-associated electric conduction bands but allows the thermally emitted electrons to fly and/or flow through ballistically. The asymmetric electron-gating function 1003 effectively allows freely emitted thermal electrons 1005 to ballistically fly predominantly from the electric conductor (emitter) 1001 through the barrier space 1004 to the electric conductor (collector) 1002 although the two electric conductors 1001 and 1002 are under the same temperature and pressure conditions. Since the barrier space 1004 is an electrical insulating space without the conventional conductor-based electrical conduction but has a unique property that allows thermal electrons to fly through ballistically, it prevents the excess thermal electrons captured by the collector 1002 from conducting back to the emitter except the minimal back emission from the collector that may be controlled by the asymmetric electron-gating function 1003. As a result, the excess thermal electrons captured by the collector 1002 may accumulate, thermally equilibrates and electrostatically distribute themselves mostly to the collector 1002 electrode surface. Similarly, the excess positive charges (“holes”) left in the emitter may also accumulate and electrostatically distribute themselves mostly to the emitter 1001 electrode surface. This results in the creation of an electric voltage potential difference across the barrier space 1004 between the emitter electrode 1001 and the collector electrode 1102, in a manner that is analogous to the creation of a membrane potential Δψ in proton-coupling bioenergetics systems as expressed in Eq. 2b.

Note, in the cases of localized excess protons, when a protonic load circuit such as an ATP synthase protonic channel/load is provided, the excess protons typically flow through the ATP synthase protonic channel across the membrane to perform work in driving ATP synthesis as illustrated in FIG. 4. Analogously, when an external electric load circuit is connected between the emitter and the collector, the excess electrons in the collector can flow through the external load circuit back to the emitter. Consequently, in this case, the excess electrons in the collector electrode will pass through an external circuit comprising an electrically conducting lead as an electric outlet 1007 (−) and an electrical load 1008 connected with another wire as electric outlet 1007 (+) back to the emitter 1001 (FIG. 13). By doing so, a portion of the environmental heat energy (thermal motion energy) associated with the thermal electrons is utilized to perform work through use of the electrical load 1008 in this example.

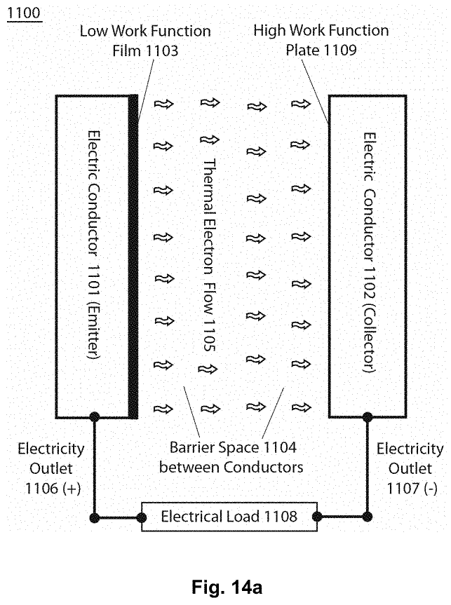

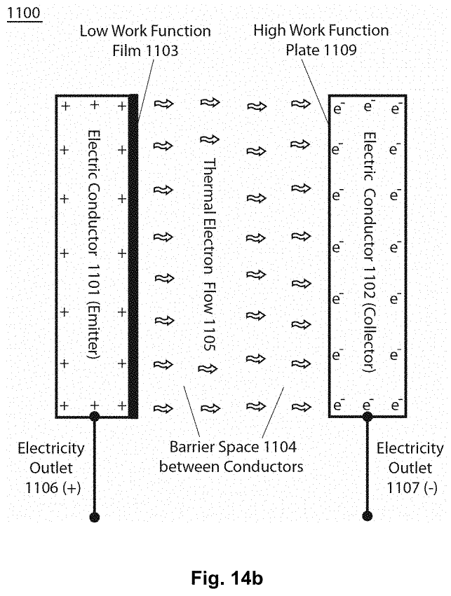

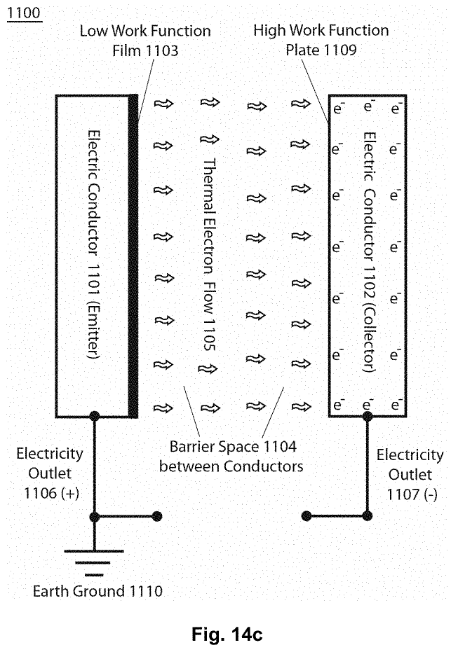

According to one of the various embodiments as shown in FIG. 14, the asymmetric electron-gating function comprise a pair of a low work function film 1103 formed on the surface of electric conductor 1101 to serve as the emitter, a high work function plate 1109 as part of electric conductor 1102 to serve as the collector, a barrier space 1104 that separates the emitter and the collector, two electrically conducting leads 1106 and 1107 that are connected with each of these electrodes 1101 and 1102 to serve as the two power terminals that may be connected with an electrical load 1108.

FIG. 14a illustrates a basic unit of an asymmetric function-gated isothermal electron power generator system 1100 comprising a barrier space 1104 such as a vacuum space that separates a pair of electric conductors 1101 and 1102: one of them has a low work function film 1103 surface and the other has a high work function plate 1109 surface. The film 1103 is made of a low work function material such as Ag—O—Cs that has a work function as low as about 0.7 eV to serve as the emitter. The barrier space 1104 is a special electric insulator space such as vacuum space that does not conduct electricity by the regular electric conduction but allow free thermal electrons 1105 to fly or flow through ballistically. Use of such barrier space 1104 and low work function film 1103 enable significant amounts of the ambient temperature thermal electrons to emit from the film surface into the barrier space 1104 and fly ballistically towards the collector that is a high work function plate 1109 such as a copper plate which has a work function as high as about 4.65 eV. At ambient temperature around 298 K, such a high work function plate 1109 practically has nearly zero emission of thermal electrons from its surface whereas it can accept the thermal electrons flying through the barrier space from the emitter 1101. After the thermal electrons 1105 from the emitter 1101 flowing ballistically across the barrier space arrive at the collector 1102, they as excess electrons will electrostatically repel each other and spread around the electric conductor 1102 (collector) surface in a way quite similar to the behavior of the excess protons in a proton conductive water body illustrated in FIG. 1c. Similarly, the excess holes (positive charges) left at the emitter will also electrostatically spread around the electrode 1101 (emitter) surface as illustrated in FIG. 14b. As a result, this creates a voltage difference between the emitter 1101 and the collector 1102. Use of this voltage difference through the terminals of electricity outlets 1107 (−) and 1106 (+) can drive an electric current through the load resistance 1108 to do electric work as shown in FIG. 14a. This conductive flow of electrons through the external load wire, better known as electricity, will continue as the excess electrons flow conductively through the external circuit back to the emitter where they will get re-emitted again for the next cycle and so on after gaining thermal motion energy from the environmental heat of the surrounding environment. This explains how the system 1100 can isothermally generate electricity by utilizing latent (existing hidden) heat from the environment.

As mentioned above, this phenomenon (FIG. 14b) is fundamentally quite similar or analogous to that of the excess protons in a water body separated by a membrane barrier with excess hydroxyl anions at the other side of the membrane as illustrated in FIG. 1 and experimentally demonstrated in FIGS. 5-11. According to the membrane potential equation (Eq. 2b) disclosed above, it is the excess proton population density resulted from the accumulation of excess protons that builds the membrane potential Δψ in proton-coupling bioenergetics systems. Analogously, it is the excess electron population density [e_{L}⁻]⁰ accumulation at the collector electrode surface resulted from the activity of the asymmetric function-gated isothermal electron-based power generator system across the emitter and the collector that builds the output voltage Vₒᵤₜₚᵤₜ, which is defined as the electrical voltage potential difference between the emitter electrode and the collector electrode for isothermal electricity production. Consequently, according to one of the various embodiments, the isothermal electricity output voltage Vₒᵤₜₚᵤₜ under the “open circuit” conditions, can be expressed as a function of the ideal effective concentration of the localized excess electrons [e_{L}⁻]⁰ at the collector electrode surface using the following equation:

$${V}_{output} = \frac{F \cdot d \cdot l \cdot {[ {e}_{L}^{-} ]}^{0}}{κ \cdot {ɛ}_{0}} \qquad [ 11 a ]$$

Where F is the Faraday constant; d is the barrier space thickness that is the distance between the emitter and the collector; κ is the barrier space dielectric constant; ε₀ is the electric permittivity; and l is the localized excess electron layer thickness.

This equation (Eq. 11a) mathematically explains how the accumulation of excess electron population density [e_{L}⁻]⁰ as a result from the capturing of thermally emitted electrons from the emitter by the collector can build the isothermal electricity output voltage Vₒᵤₜₚᵤₜ Consequently, the excess electrons in the collector electrode with such an output voltage Vₒᵤₜₚᵤₜ can drive an electric current through an external circuit, which comprises an electric outlet 1107 (−) wire connected with an electrical load 1108 that is connected with another electric wire as electric outlet 1106 (+) back to the emitter 1101 as shown in FIG. 14a. By doing so, a portion of the environmental heat energy (thermal motion energy) associated with the thermal electrons is utilized to perform work through use of an electrical load 1108 in this example.

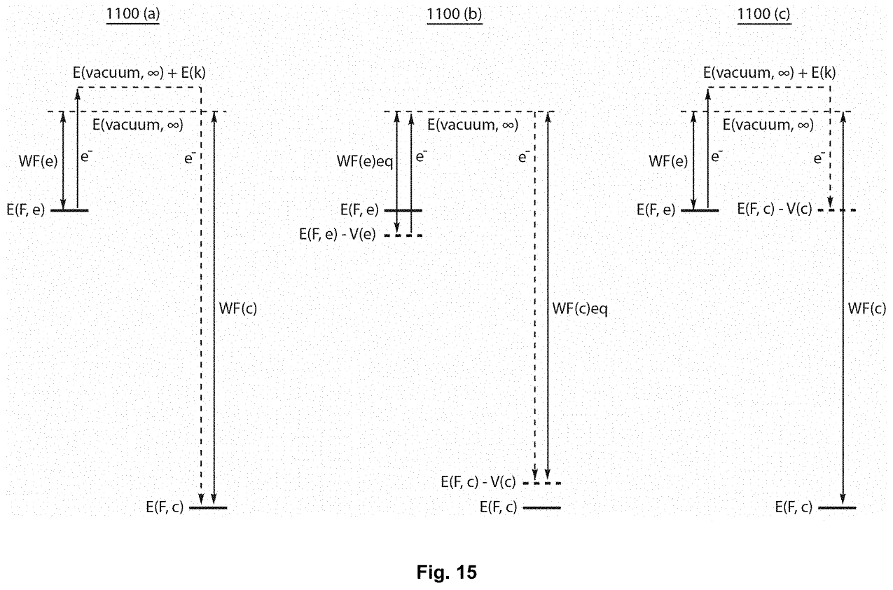

FIG. 15 presents the energy diagrams of the asymmetric function-gated isothermal electron power generator system 1100. As shown in FIG. 15a (left), the work function (WF(e)) of the emitter 1101 (FIG. 14a) is the energy level difference between the Fermi energy level (E(F, e)) of the emitter and the vacuum energy level (E(vacuum, ∞) of a free electron that is considered “infinitely” (∞) far away from the emitter and collector surfaces; while the work function (WF(c)) of the collector 1102 is the difference between the collector's Fermi energy level (E(F, c)) and the vacuum energy level (E(vacuum, ∞). As mentioned before, it is a preferred practice to employ an emitter with a work function as low as possible such as about 0.7 eV so that significant amounts of the ambient temperature thermal electrons can emit from the emitter surface into the vacuum barrier space 1104 and fly ballistically with kinetic energy (E(k)) towards the collector 1109 that has a work function (WF(c)) much larger than that of the emitter (WF(e)). On the other hand, essentially no ambient-temperature thermal electrons can emit from the high work function collector surface into the vacuum barrier space 1104 since the work function of the collector (WF(c)) is so big (for example, above 2.0 eV) that the ambient-temperature thermal electrons are essentially not able to escape from the collector surface. Consequently, there are statistically many more free thermal electrons 1105 flying from the emitter 1101 into the collector 1102 than that in the opposite direction. After the emitted electrons arriving at the collector 1102, they will thermally equilibrate with the environment and electrostatically result in the creation of a voltage at the collector (V(c)) as expressed in Eq. 11a that can drive an electric current through an external electric load 1108 back to the emitter 1101. This completes a cycle of the asymmetric function-gated thermal electron power generation process and gets ready for the next cycles of thermal electron emission and collection as shown in FIG. 14a.

When the asymmetric function-gated isothermal electron power generator system 1100 is under its “open circuit” condition (such as when the electric load 1108 is removed) as shown in FIG. 14b, as mentioned before, the activity of the asymmetric function-gated thermal electron power generation process will result in the accumulation of excess electrons in the collector thus generating a negative voltage V(c) there; Meanwhile, this may also result in the accumulation of excess positive charges at the emitter thus generating a positive voltage V(e) there. The negative voltage V(c) at the collector will push up its effective Fermi level to that of E(F, c) plus the absolute value of V(c) (labeled as “E(F, c)-V(c)” in the 1100 (b) of FIG. 15); whereas the positive voltage V(e) at the emitter will push down its effective Fermi level to a lower level of (E(F, e)-V(e)) as shown in the 1100 (b) of FIG. 15 (middle). Consequently, under the “open circuit” condition, the effective work function of the emitter at the equilibrated state (WF(e)eq) is increased by the product e·V(e) of the election unit charge e and V(e) to a higher value (WF(e)+e·V(e)) while the effective work function of the collector (WF(c)eq) is decreased by the absolute value of e·V(c) to a lower (smaller) value (WF(c)+e·V(c)). The larger (higher) effective work function of the emitter (WF(e)+e·V(e)) will reduce and eventually pretty much cut off the ambient-temperature electron emission at the emitter 1101 and consequently the accumulation of positive charges at the emitter will then stop, resulting in an equilibrated value of V(e) as shown in FIG. 15b.

According to one of the various embodiments, it is a preferred practice to ground the emitter with an Earth ground 1110 at the electricity outlet 1106 (+) terminal as shown in FIG. 14c to prevent the accumulation of positive charges there. When the emitter is “Earth grounded” (V(e)=0), the effective work function of the emitter will be retained at the initial value of WF(e) even when the 1100 system is under the “open circuit” condition. In this way, the ambient-temperature electron emission at the emitter 1101 will continue until the effective Fermi level of the collector (E(F, c)-V(c)) will rise so much by the absolute value of V(c) that will match at the same level of the emitter E(F, e) with WF(e) as shown in the 1100(c) of FIG. 15 (right). At this point, the back emission flow of the ambient-temperature electrons from the collector 1102 to the emitter 1101 will cancel the flow of the ambient-temperature electrons from the emitter 1101 to the collector 1102 at an equal rate. In this case, at its equilibrium state, V(c) will equal to the difference between the collector work function WF(c) and emitter work function WF(e) over the electronic unit charge (e for electron e).

This asymmetric function-gated isothermal electron power generator system 1100 (FIG. 14) is fundamentally different from the conventional temperature gradient-driven thermionic converter reported previously by Hatsopoulos and Gyftopoulos 1973 (Thermionic Energy Conversion, Volume I: Processes and Devices, The MIT Press, Cambridge, Mass., and London, England). The conventional thermionic converter converts heat to electricity by boiling electrons from a very hot emitter surface (˜2000 K) across a small inter electrode gap (<0.5 mm) to a cooler collector surface (˜1000 K), which requires a large temperature gradient and clearly is not an isothermal operation in contrast to the isothermal electricity generation disclosed in the present inventions. Since the thermionic converter is a form of heat engine which runs by using a temperature gradient, it is believed to be limited by the Carnot efficiency, at best. In the conventional temperature gradient-driven thermionic converter reported by King et al 2004 (Sandia Report, SAND2004-0555, Unlimited Release, Sandia National Laboratory, Albuquerque, N. Mex.) and by Chou 2014 (Discovering Low Work Function Materials For Thermionic Energy Conversion, PhD Dissertation, Stanford University, California), a high work function electrode is typically used as the emitter that is heated up by a high temperature heat source while a low work function electrode is used as the collector that is cooled by a cold heat sink so that the conventional thermionic electricity generation is believed to be driven by the temperature difference between the heated emitter and the cooled collector in “following the second law of thermodynamics”.

In contrast, for an isothermal electricity generator system such as the one illustrated in FIG. 14c, it is preferred to use a special low work function conductor as the emitter electrode 1101 while the collector electrode 1102 is selected to have a higher work function predominately from the nuclear (positive) charge force. More importantly, both the emitter 1101 and the collector 1102 can be used at the same ambient temperature (isothermal conditions) without requiring the use of a significant temperature gradient between the emitter and the collector. Consequently, the isothermal electron power generator system which isothermally extracts latent heat energy from the environment for generating useful electricity perfectly follows the first law of thermodynamics but without being constrained by the second law of thermodynamics owning to the use of the special asymmetric function-gated mechanisms.

In the conventional temperature gradient-driven thermionic converter, a conducting electrode (emitter) is heated to high temperatures so that it emits electrons (Wanke et al 2017 MRS Bulletin 42: 518-524). These thermionic electrons overcome the electrode's work function and generate a thermionic emission current. It typically requires the emitter being heated by using an external energy/heat source such as focused solar irradiation, intensified chemical combustion, or nuclear decay reaction heat to a temperature as high as 2000K while the collector is cooled to below about 600K using a heat sink (Sandia Report, SAND2004-0555). Air-breathing chemical heat sources, such as common hydrocarbon burners, cannot achieve the desired thermionic temperatures (˜2000K) unless substantial air-preheat is used. That is, the thermionic converter operation is based on an exceptionally high temperature at the emitter with a large temperature difference between the two electrodes (thermionic emitter and collector). The elevated high temperatures required by the thermionic converter impose formidable technical problems concerning the structure of the fuel elements and the means of transferring heat to the converters. The Carnot efficiency here is believed to represent the ultimate efficiency limit (Khalid et al 2016 IEEE Transactions on Electron Devices 63: 2231-2241). In contrast, the asymmetric function-gated isothermal electron power generator system disclosed in the present invention does not require such an elevated high temperature and is not constrained by the Carnot efficiency, since it can generate electricity by isothermally utilizing the ambient temperature latent heat energy from the surrounding environment without requiring any of such energy-intensive heating and/or cooling energy resources.

According to one of the various embodiments in accordance with the present invention, the asymmetric electron-gating function 1003 (FIG. 13) that comprises the utilization of low work function emitter 1103 (FIG. 14a) typically coated on the surface of an electric conductor 1101, which is able to emit thermal electrons even at the ambient temperature (such as 293 K (20° C.)) and the utilization of higher work function collector 1109 on an electric conductor plate 1102 surface under the ambient temperature conditions that essentially will not emit electrons but be able to collect the thermal electrons from the emitter 1103. It is this asymmetric electron-gating function that enables the flow of thermal electrons 1105 through the vacuum barrier space 1104 from the emitter 1103 to the collector 1109 under the isothermal conditions, generating an electricity output with a voltage difference across the two outlets 1106 (+) and 1007(−) without being constrained by the second law of thermodynamics. Therefore, this asymmetric function-gated isothermal electron power generator system 1100 (FIG. 14) represents a special Anti-Second-Law energy technology function that is capable of energy renewal by extracting the latent (existing hidden) heat energy from the ambient environment through the use of thermal electrons associated with the emitter and the collector and converting it to useful energy in the form of electricity under the isothermal conditions. Fundamentally, this is somewhat similar to the Anti-Second-Law energy function disclosed previously with the systems of localized protons above.

Previous study suggested that the conventional thermionic generators could be effective, but only at temperatures above 1000K (Hishinuma et al 2001 Applied Physics Letters 78: 2572-2574). In contrast, the asymmetric function-gated isothermal electron power generator system can operate isothermally at nearly any temperatures from a freezing temperature such as 253K (−20° C.), to ambient temperatures around 293K (20° C.), to an elevated temperature as high as both above and/or below 1000K where the conventional thermionic generators still cannot effectively operate. According to one of the various embodiments in accordance with the present invention, an asymmetric function-gated isothermal electricity generator system is designed to isothermally operate at a temperature or temperature range selected from a group consisting of 193K (−80° C.), 200K (−73° C.), 210K (−63° C.), 220K (−53° C.), 230K (−43° C.), 240K (−33° C.), 250K (−23° C.), 260K (−13° C.), 270K (−3° C.), 273K (0° C.), 278K (5° C.), 283K (10° C.), 288K (15° C.), 293K (20° C.), 298K (25° C.), 303K (30° C.), 308K (35° C.), 313K (40° C.), 318K (45° C.), 323K (50° C.), 328K (55° C.), 333K (60° C.), 338K (65° C.), 343K (70° C.), 348K (75° C.), 353K (80° C.), 363K (90° C.), 373K (100° C.), 383K (110° C.), 393K (120° C.), 403K (130° C.), 413K (140° C.), 423K (150° C.), 433K (160° C.), 453K (180° C.), 473K (200° C.), 493K (220° C.), 513K (240° C.), 533K (260° C.), 553K (280° C.), 573K (300° C.), 623K (350° C.), 673K (400° C.), 723K (450° C.), 773K (500° C.), 823K (550° C.), 873K (600° C.), 923K (650° C.), 973K (700° C.), 1073K (800° C.), 1173K (900° C.), 1273K (1000° C.), 1373K (1100° C.), 1473K (1200° C.), and/or within a range bounded by any two of these values. The words “to isothermally operate” here means that both the emitter and collector are placed at the same temperature and no temperature difference between the emitter and collector is required for the asymmetric function-gated isothermal electricity generation to run in accordance with one of the various embodiments of the present invention.

According to one of the various embodiments, it is critically important to properly select a special low work function conductor to serve as the emitter with consideration of its operating environmental temperature conditions. For example, for an asymmetric function-gated thermal electron power generator system that is designed to operate at a room temperature (around 25° C.), the work function of the emitter is preferably selected to be less than 1.0 eV, more preferably less than 0.8 eV, even more preferably less than 0.7 eV or 0.6 eV, and most preferably less than 0.5 eV. For an asymmetric function-gated isothermal electron power generator system designed to isothermally operate at a higher environmental temperature such as 35° C., 40° C., 50° C., 60° C., 80° C., 100° C., 120° C., 150° C., 200° C. and/or within a range bounded by any two of these values, somewhat higher work function materials may also be selected for use as the emitters. On the other hand, when the intended isothermally operating temperature is significantly lower, such as, at 15° C., 10° C., 5° C., 0° C., −5° C., −10° C., −15° C., −20° C., −30° C., −50° C. and/or within a range bounded by any two of these values, exceptionally low work function materials should be selected for use as the emitters.

According to one of the various embodiments, depending on a given specific application and its associated temperature conditions, system compositions, and the properties of the electrode materials and barrier space such as its thickness, capacitance and other physical chemistry properties, the work function of the emitters for the purpose of extracting environmental heat to generate electricity may be selected from the group consisting of 0.2 eV, 0.3 eV, 0.4 eV, 0.5 eV, 0.6 eV, 0.7 eV, 0.8 eV, 0.9 eV, 1.0 eV, 1.1 eV, 1.2 eV, 1.3 eV, 1.4 eV, 1.5 eV, 1.6 eV, 1.7 eV, 1.8 eV, 1.9 eV, 2.0 eV, 2.1 eV, 2.2 eV, 2.4 eV, 2.6 eV, 2.8 eV, 3.0 eV and/or within a range bounded by any two of these values.

According to one of the various embodiments, the collector electrode 1102 is preferable to have a work function higher than that of its pairing emitter 1101 (FIG. 14) so that no appreciable isothermal electron emission occurs at the collector surface. Depending on a given specific application and its associated temperature conditions, system compositions, and the properties of the electrode materials and barrier space such as its thickness, capacitance and other physical chemistry properties, the work function of the collectors for the purpose of extracting environmental heat to generate isothermal electricity is selected from the group consisting of 1.0 eV, 1.1 eV, 1.2 eV, 1.3 eV, 1.4 eV, 1.5 eV, 1.6 eV, 1.7 eV, 1.8 eV, 1.9 eV, 2.0 eV, 2.1 eV, 2.2 eV, 2.4 eV, 2.6 eV, 2.8 eV, 3.0 eV, 3.2 eV, 3.4 eV, 3.6 eV, 3.8 eV, 4.0 eV, 4.2 eV, 4.4 eV, 4.6 eV, 4.8 eV, 5.0 eV, 5.5 eV, 6.0 eV, and/or within a range bounded by any two of these values.

As mentioned before, the work function represents the energy barrier for an electron at the Fermi level from escaping the solid (such as the metal conductor) to free space. The work function commonly comprises two components: a bulk component and a surface component. The dominant one is the bulk component which corresponds to the chemical potential that derives from the electronic density and density of states with relation to the nuclear (positive) charge force in the solid. The surface component (also known as the surface dipole component) originates with a redistribution of charges at the surface of a metal, which give rise to the surface dipole that is generally resulted from the “spill out” of electrons into vacuum over some small distance (Angstroms), creating negative sheet of charges outside the solid and leaving a positive sheet of uncompensated metal ions in the surface and sub-surface atomic planes. It is this double sheet of charges (surface dipoles) that create a potential step which raises the electron potential just out the surface, effectively also raising the electron vacuum energy level at the emitter electrode surface Evac (S). This surface dipole-associated component may correspond to the energy difference between the Evac (S) (the vacuum energy level at the emitter electrode surface) and the Evac (∞) in vacuum space far away from the surface. The surface dipole-associated negative charge could repel an electron away the electrode. Consequently, the electrons leaving the emitter surface could be accelerated towards the collector by this repulsive force from the emitter's surface dipole, which may be beneficial to the isothermal electricity generation. On the other hand, if the collector also has a surface dipole-associated negative charge component that could potentially impede the reception of the electrons emitted from the emitter by repelling them away from the collector surface. Therefore, according to one of the various embodiments, it is a preferred practice to use a collector electrode that has no or minimized surface dipole-associated negative charge component. Alternatively, if there is the surface dipole-associated negative charge component on the collector surface, it needs to be nearly equal to or smaller than that of the emitter surface for the isothermal electricity generator to more efficiently operate. That is, it is beneficial to use a work function that originates predominately from the nuclear (positive) charge force with no or minimal surface dipole-associated negative charge force for the collector to better collect the electrons emitted from the emitter.

It is critically important to properly select a special low work function conductor as the emitter while the collector should have a higher work function predominately from the nuclear (positive) charge force. Table 6 lists various materials with known work function (eV) values, which may be considered for selection to use in making of the emitters and/or collectors in accordance with one of the various embodiments of the present invention.

TABLE 6
Examples of various materials with known work function (eV) that may be considered
for selection to use in making of the emitters and/or the collectors according to one of the various
embodiments in the present invention.

| Work Function | | |
|---|---|---|
| (eV) | Material | Special Note |
| 0.3 − 1.0 | K—O/Si(100) | Wu et al 1999 Phys Rev B, 60: 17102-17106 |
| 0.5 − 1.2 | Ag—O—Cs | Depending on experimental operating conditions |
| 0.6 | C12A7:e− | Predicted by Rand et al 2015 IEEE Transactions on |
| | | Plasma Science, 43: 190-194 |
| 0.7 − 0.8 | K on WTe2 | Kim et al 2017 Journal of Physics-Condensed |
| | | Matter, 29, 315702 (8pp) |
| 0.9 | P-doped diamond | Koeck et al 2009 Diam. Relat. Mater. 18: 789-791 |
| <1 | Ca₂₄Al₂₈O₆₄ | Toda et al 2004 Adv. Mater. 16: 685 |
| 1.01 ± 0.05 | Cs/O doped | Yuan et al 2015 Nano Letters 15: 6475-6480 |
| | graphene | |
| 1.07 | Sri₁₋ₓBaₓVO₃ | Patent Application Pub No. US2017/0207055 |
| 1.1 | Cs₂O-coated Ag | Based on the preliminary experimental study by the |
| | plate surface | inventor (Lee, J W) |
| 1.2 | Ba-coated SiC | Lee et al 2014 Journal of Microelectromechanical |
| | | Systems 23: 1182-1187 |
| 1.35 | K—O on silicon | Morini et al 2014 Phys. Status Solidi A 211: 1334- |
| | | 1337 |
| 1.4 | O—Ba on W | Zagwijn et al 1997 Appl. Surf. Sci. 111: 35 |
| 1.4 | Cs on Pt metal | Hishinuma et al 2001 Applied Physics Letters 78: |
| | | 2572-2574 |
| 1.95 | Cs (Caesium) | |
| 2.261 | Rb (Rubidium) | |
| 2.29 | K (Potassium) | |
| 2.36 | Na (Sodium) | |
| 2.52 − 2.70 | Ba (Barium) | |
| 2.7 | Sm (Samarium) | |
| 2.9 | Li (Lithium) | |
| 3.00 | Tb (Terbium) | |
| 3.2 | Nd (Neodymium) | |
| 3.40 ± 0.06 | Al metal | Zhou et al 2012 Science 336: 327-332 |
| 3.63 − 4.9 | Zn (Zinc) | |
| 3.66 | Mg (Magnesium) | |
| 4.06 − 4.26 | Al (Aluminum) | |
| 4.08 | Cd (Cadmium) | |
| 4.1 | Mn (Manganese) | |
| 4.10 ± 0.15 | Ag(110) | Derry et al 2015 J. Vac. Sci. Technol. A 33(6): |
| | | 060801-9; dx.doi.org/10.1116/1.4934685 |
| 4.23 ± 0.13 | Al(110) | 2015 J. Vac. Sci. Technol. A 33(6): 060801-9 |
| 4.25 | Pb (Lead) | |
| 4.26 ± 0.06 | ZnO metal oxide | Zhou et al 2012 Science 336: 327-332 |
| 4.26 − 4.74 | Ag (Silver) | |
| 4.31 ± 0.18 | Al(100) | 2015 J. Vac. Sci. Technol. A 33(6): 060801-9 |
| 4.32 ± 0.06 | Al(111) | 2015 J. Vac. Sci. Technol. A 33(6): 060801-9 |
| 4.32 | Ga (Gallium) | |
| 4.32 − 5.22 | W (Tungsten) | |
| 4.33 | Ti (Titanium) | |
| 4.36 ± 0.05 | Ag(100) | 2015 J. Vac. Sci. Technol. A33(6): 060801-9 |
| 4.36 − 4.95 | Mo (Molybdenum) | |
| 4.37 ± 0.24 | Mo(111) | 2015 J. Vac. Sci. Technol. A 33(6): 060801-9 |
| 4.42 | Sn (Tin) | |
| 4.42 ± 0.14 | Polyaniline film | Abdulrazzaq et al 2015 RSC Adv. 5: 33-40 |
| 4.44 ± 0.03 | W(111) | 2015 J. Vac. Sci. Technol. A 33(6): 060801-9 |
| 4.46 ± 0.11 | Mo(100) | 2015 J. Vac. Sci. Technol. A 33(6): 060801-9 |
| 4.53 ± 0.02 | Mo(100) crystal | Surface Science 43 (1974) 275-292 |
| 4.53 ± 0.07 | Ag(111) | 2015 J. Vac. Sci. Technol. A 33(6): 060801-9 |
| 4.53 − 5.10 | Cu (Copper) | |
| 4.56 ± 0.10 | Cu(110) | 2015 J. Vac. Sci. Technol. A 33(6): 060801-9 |
| ~4.6 | Graphite | Yuan et al 2015 Nano Letters 15: 6475-6480 |
| 4.60 ± 0.06 | Ag metal | Zhou et al 2012 Science 336: 327-332 |
| 4.60 ± 0.06 | Graphene | Zhou et al 2012 Science 336: 327-332 |
| 4.60 − 4.85 | Si (Silicon) | |
| 4.60 ± 0.33 | Fe(100) | 2015 J. Vac. Sci. Technol. A 33(6): 060801-9 |
| 4.62 ± 0.06 | ITO metal oxide | Zhou et al 2012 Science 336: 327-332 |
| 4.66 | 2-dimensional | Zhou et al 2016 Nanotechnology 27 (2016) 344002 |
| | nickel | (7pp) |
| 4.67 − 4.81 | Fe (Iron) | |
| 4.68 ± 0.06 | FTO metal oxide | Zhou et al 2012 Science 336: 327-332 |
| 4.70 ± 0.06 | W(100) | 2015 J. Vac. Sci. Technol. A 33(6): 060801-9 |
| 4.71 | Ru (Ruthenium) | |
| 4.72 ± 0.13 | Ni(110) | 2015 J. Vac. Sci. Technol. A 33(6): 060801-9 |
| 4.73 ± 0.10 | Cu(100) | 2015 J. Vac. Sci. Technol. A 33(6): 060801-9 |
| 4.81 ± 0.29 | Fe(111) | 2015 J. Vac. Sci. Technol. A 33(6): 060801-9 |
| 4.84 ± 0.07 | W(211) | 2015 J. Vac. Sci. Technol. A 33(6): 060801-9 |
| 4.86 ± 0.21 | Rh(110) | 2015 J. Vac. Sci. Technol. A 33(6): 060801-9 |
| 4.90 ± 0.02 | Cu(111) | 2015 J. Vac. Sci. Technol. A 33(6): 060801-9 |
| 4.90 ± 0.06 | PEDOT:PSS | Zhou et al 2012 Science 336: 327-332 |
| 4.92 ± 0.05 | Mo(110) | 2015 J. Vac. Sci. Technol. A 33(6): 060801-9 |
| 4.98 | Rh (Rhodium) | |
| 5 | Co (Cobalt) | |
| ~5 | C (Carbon) | |
| 5.00 − 5.67 | Ir (Iridium) | |
| 5.04 − 5.35 | Ni (Nickel) | |
| 5.07 ± 0.04 | Fe(110) | 2015 J. Vac. Sci. Technol. A 33(6): 060801-9 |
| 5.07 ± 0.20 | Pd(110) | 2015 J. Vac. Sci. Technol. A 33(6): 060801-9 |
| 5.10 ± 0.10 | Au metal | Zhou et al 2012 Science 336: 327-332 |
| 5.12 − 5.93 | Pt (Platinum) | |
| 5.16 ± 0.22 | Au(110) | 2015 Vac. Sci. Technol. A 33(6): J. 060801-9 |
| 5.17 ± 0.11 | Ni(100) | 2015 J. Vac. Sci. Technol. A 33(6): 060801-9 |
| 5.22 ± 0.31 | Au(100) | 2015 J. Vac. Sci. Technol. A 33(6): 060801-9 |
| 5.22 − 5.60 | Pd (Palladium) | |
| 5.24 ± 0.07 | Ni(111) | 2015 J. Vac. Sci. Technol. A 33(6): 060801-9 |
| 5.30 ± 0.15 | Rh(100) | 2015 J. Vac. Sci. Technol. A 33(6): 060801-9 |
| 5.33 ± 0.06 | Au(111) | 2015 J. Vac. Sci. Technol. A 33(6): 060801-9 |
| 5.42 ± 0.32 | Ir(100) | 2015 J. Vac. Sci. Technol. A 33(6): 060801-9 |
| 5.44 ± 0.14 | W(110) | 2015 J. Vac. Sci. Technol. A 33(6): 060801-9 |
| 5.46 ± 0.09 | Rh(111) | 2015 J. Vac. Sci. Technol. A 33(6): 060801-9 |
| 5.48 ± 0.23 | Pd(100) | 2015 J. Vac. Sci. Technol. A 33(6): 060801-9 |
| 5.53 ± 0.13 | Pt(110) | 2015 J. Vac. Sci. Technol. A 33(6): 060801-9 |
| 5.60 | Pt metal | Hishinuma et al 2001 Applied Physics Letters 78: |
| | | 2572-2574 |
| 5.67 ± 0.12 | Pd(111) | 2015 J. Vac. Sci. Technol. A 33(6): 060801-9 |
| 5.67 ± 0.14 | Pt(100)[5X1] | 2015 J. Vac. Sci. Technol. A 33(6): 060801-9 |
| 5.75 ± 0.13 | Pt(100)[1X1] | 2015 J. Vac. Sci. Technol. A 33(6): 060801-9 |
| 5.78 ± 0.04 | Ir(111) | 2015 J. Vac. Sci. Technol. A 33(6): 060801-9 |
| 5.91 ± 0.08 | Pt(111) | 2015 J. Vac. Sci. Technol. A 33(6): 060801-9 |
| 5.93 | Os (Osmium) | |
| 5.95 ± 0.25 | Ir(100)[5X1] | 2015 J. Vac. Sci. Technol. A 33(6): 060801-9 |
| 5.97 ± 0.23 | Ir(100)[X1] | 2015 J. Vac. Sci. Technol. A 33(6): 060801-9 |
| 6.6 | MoO₃ | Appl. Phys. Lett. 105, 222110 (2014) |

According to one of the various embodiments in accordance with the present invention, it is preferred practice to use a special low work function conductor as the emitter electrode while the collector electrode should have a high work function predominately from the nuclear (positive) charge force.

According to one of the various embodiments, the emitter is a layer or film of a special lower work function material 1103 coated on a conductive electrode 1101 while the collector 1109 is a film of higher work function coated on conductive electrode 1102 and/or is simply a plate of higher-work-function conductor. Depending on a given specific isothermal electricity generation application and its associated operating temperature conditions, the emitter material is selected from a group consisting of Ag—O—Cs, Cs₂O-coated Ag plate surface, K—O/Si(100), C12A7:e−, K on WTe2, P-doped diamond, P-doped diamond, Ca₂₄Al₂₈O₆₄, Cs/O doped graphene, Sr₁₋ₓBaₓVO₃, Ba-coated SiC, O—Ba on W, Cs on Pt metal and combinations thereof. Meanwhile, the collector material is selected from a group consisting of platinum (Pt) metal, silver (Ag) metal, gold (Au) metal, copper (Cu) metal, molybdenum (Mo) metal, aluminum (Al) metal, tungsten, rhenium, molybdenum, niobium, nickel, graphene, graphite, polyaniline film, ZnO metal oxide, ITO metal oxide, FTO metal oxide, 2-dimensional nickel, PEDOT:PSS, protonated-polyaniline film and combinations thereof.

According to one of the various embodiments, the materials for making the electric conductors 1191 and 1102 that support the emitter and/or collector, and that may also directly serve as the collector are selected from the group consisting of: heat-conducting electric conductors, heat-conducting metallic conductors, refractory metals, metal alloys, stainless steels, aluminum, copper, silver, gold, platinum, molybdenum, conductive MoO₃, tungsten, rhenium, molybdenum, niobium, nickel, titanium, graphene, graphite, heat-conducting electrically conductive polymers, polyaniline film, protonated-polyaniline film and combinations thereof.

According to one of the various embodiments, it is a preferred practice to employ a conductor with no or minimized surface dipole-associated work function component to serve as a collector electrode to facilitate the collection of the electrons from the emitter. For example, nonpolar organic conductors typically have no significant “spilling” of electrons at the surface and can thus be selected to use as a collector electrode.

A major problem that has been hindering the performance of the conventional thermionic converter is the formation of the static electron space-charge clouds in the inter electrode space (Physics of Plasmas 21, 023510 (2014); doi: 10.1063/1.4865828). This “space charge problem” is minimized in the asymmetric function-gated isothermal electricity generation system (FIG. 14), for example, by its design to operate at a significantly lower current density (J) across the interelectrode space (often in a range from sub Amp/cm² to no more than a few Amp/cm²) than that of the conventional thermionic converter which typically is on the order of over 10-100 A/cm² (temperatures 1000-2000 K). In the conventional thermionic converter, as electrons are emitted into the interelectrode space with such a high current density (J), they can repel each other and tend to be pulled back into the emitter, which now has a positive charge after having lost some electrons, and to form a cloud of negative charges close to the emitter surface. This results in what is called the space charge effect, which later on repels the additional emitted electrons away from the collector, thus reducing the current transferred to the collector. The space charge effect also creates an additional potential barrier to electron emission. Only those electrons with sufficient kinetic energy are able to reach the collector. Therefore, according to one of the various embodiments, the “space charge problem” is minimized by a number of ways selected from the group consisting of: 1) by operating the isothermal electricity generation system (FIG. 14) naturally at a relatively lower current density (J) across the interelectrode space (in a range from sub Amp/cm² to no more than a few Amp/cm²); 2) by grounding the emitter as shown in FIG. 14c; 3) by using a capacitor with the emitter and/or the collector, 4) by minimizing the interelectrode space distance between the emitter and the collector to the scales of micrometers and/or nanometers; 5) by using the gravity to facilitate the thermal electron flow from the emitter to the collector; 6) by using positively charged molecular structures such as protonated amine groups on the collector surface; and combinations thereof.

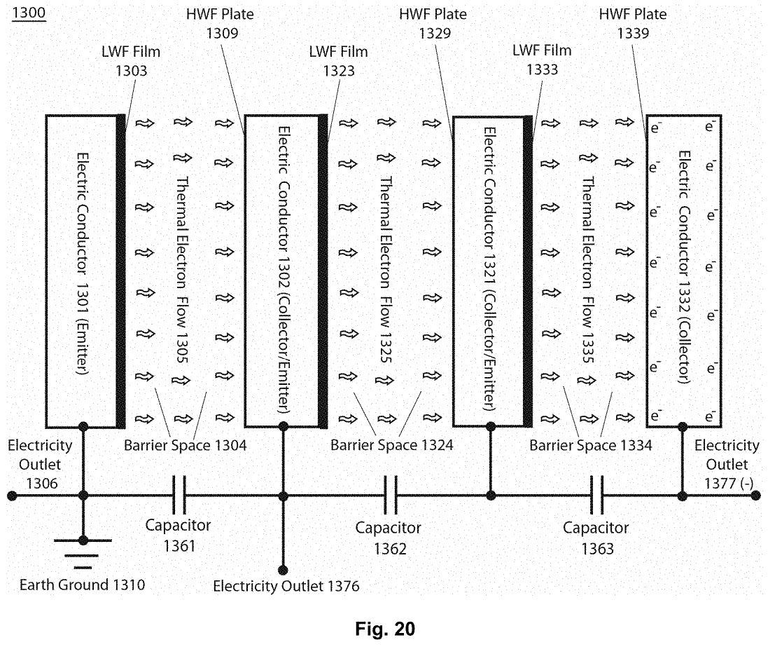

According to one of the various embodiments, a series of capacitors can be used across each of pairs of the emitters and the collectors with the isothermal electricity outlets (illustrated in the example of FIG. 20 below) to increase the capacitance across each pair of the emitter and collector to improve the stability and efficacy of the isothermal electricity generator system.

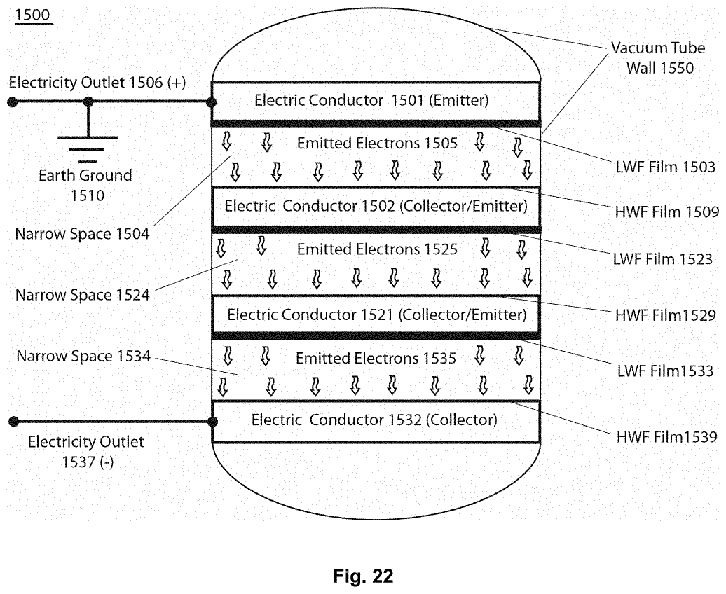

According to one of the various embodiments, the capacitance across each pair of the emitter and collector is increased by properly narrowing the space separation distance between the emitter surface and the collector surface (illustrated in the example of FIG. 22 below) to improve the stability and efficacy of the isothermal electricity generator system. A smaller and highly evacuated interelectrode space gap distance can limit the number of electrons travelling within it. Excessive numbers of electrons in transit will form an electron cloud, causing decreased efficiency due to the space charge effect. Therefore, it is a preferred practice to properly minimize the separation distance between the emitter surface and the collector surface to increase capacitance and limit the formation of the static electron space-charge clouds in the inter electrode space for enhanced isothermal electricity generation.

On the other hand, the barrier space separation distance between the emitter surface and the collector surface should be big enough (somewhat larger than the electron tunneling distance (2 or 3 nm)) to avoid electricity current leaking loss due to the possible electron tunneling. Considering the surface of a metal as a two-dimensional system, electrons cannot escape, but due to “barrier penetration”, the electron density of a metal actually extends outside the surface of the metal. The distance outside the surface of the metal at which the electron probability density drops to 1/1000 of that just inside the metal is on the order of 0.1 to 1 nanometer (nm) for electron tunneling which is strongly dependent on the distance. The electron tunneling distance is also depending on the property of the materials and barrier space. For example, electron transfer and tunneling can occur between the metal centers in the respiratory enzymes, typically over distances up to 20 or 30 Å (2010 Laser Phys. 20(1): 125-138). It is also known that biological lipid bilayer membrane with a thickness about 4 nm works well as an electric insulating barrier space with a membrane potential voltage difference of about 200 mV. In certain cases, larger barrier space gaps may be also desirable such as for ease of fabrication and certain mechanical operations. Therefore, depending on a given specific application and its associated temperature conditions, system compositions, and the properties of the electrode materials and barrier space, the inter electrode space separation distance (gap size d) across a pair of emitter and collector according to one of the various embodiments is selected from the group consisting of 2 nm, 3 nm, 4 nm, 5 nm, 6 nm. 7, nm, 8 nm, 9 nm, 10 nm, 12 nm, 14 nm, 16 nm, 18 nm, 20 nm, 25 nm, 30 nm, 35 nm, 40 nm 45 nm, 50 nm, 60 nm, 70 nm, 80 nm, 100 nm, 120 nm, 140 nm 160 nm, 180 nm, 200 nm, 250 nm, 300 nm, 500 nm, 600 nm, 700 nm, 800 nm, 900 nm, 1000 nm, 1.2 μm, 1.4 μm, 1.6 μm, 1.8 μm, 2.0 μm, 2.5 μm, 3.0 μm, 3.5 μm, 4.0 μm, 4.5 μm, 5.0 μm, 6.0 μm, 7.0 μm, 9.0 μm, 10 μm, 12 μm, 14 μm, 16 μm, 18 μm, 20 μm, 25 μm, 30 μm, 35 μm, 40 μm, 45 μm, 50 μm, 60 μm, 70 μm, 80 μm, 90 μm, 100 μm, 120 μm, 140 μm, 160 μm, 180 μm, 200 μm, 250 μm, 300 μm, 400 μm, 500 μm, 600 μm, 700 μm, 800 μm, 900 μm, 1000 μm, 1.2 mm, 1.4 mm, 1.6 mm, 1.8 mm, 2.0 mm, 2.5 mm, 3.0 mm, 4.0 mm, 5.0 mm, 6.0 mm, 7.0 mm, 8.0 mm, 9.0 mm, 10 mm, 12 mm, 15 mm, 20 mm, 30 mm, 40 mm, 50 mm, 60 mm, 80 mm, 100 mm and/or within a range bounded by any two of these values.

According to one of the various embodiments, a barrier space composition is selected from the group consisting of vacuum space, semi-vacuum space, gaseous space, inertial gas space, special gas space, ballistic-electron-permeable porous material space, perforated two-dimensional (2D) materials, perforated insulator film such as perforated Teflon film, and combinations thereof. When considering to utilize certain special gaseous space, attention should be paid to avoid possible side reactions associated with the gas molecules and properties of the electrodes and space barrier compositions and materials when the electric field formed across the inter electrode space during the isothermal electricity generation could be high enough to cause certain side effects such as the undesirable current leaking, plasma or radical species formation, and O₃ generation if the gaseous space containing O₂ gas. For many of the applications, it is a preferred practice to use vacuum space as the inter electrode space barrier 1104 (FIG. 14). Furthermore, it is also valuable to utilize perforated two-dimensional (2D) materials such as perforated thin insulator film such as perforated Teflon and certain plastic films that allow thermal electrons to ballistically fly through with minimal absorption coefficient. The masses of thin perforated insulator films can be extremely small, making them attractive for mobile applications.

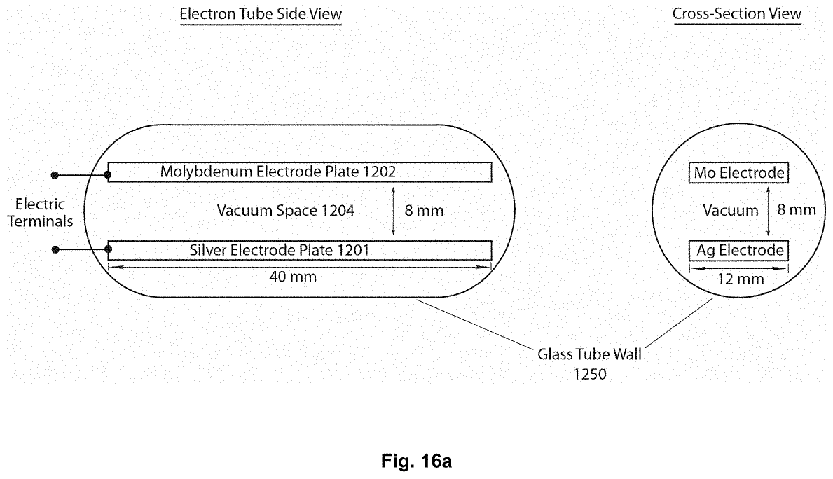

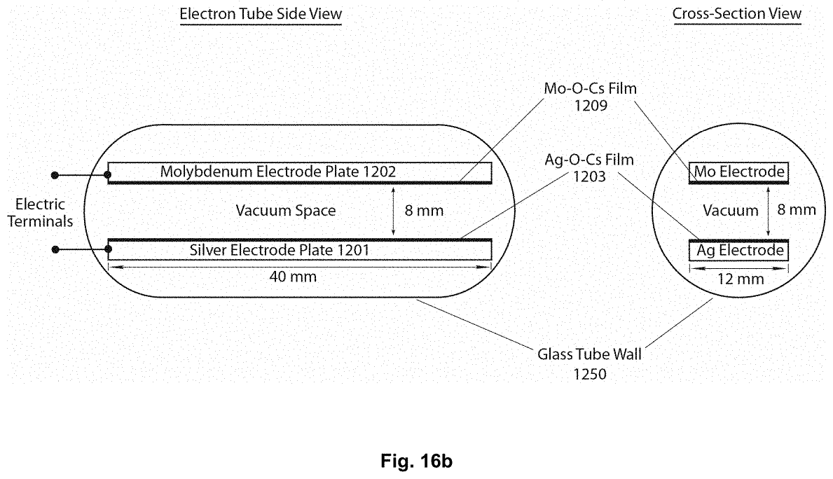

According to one of the various embodiments, emitter(s) and collector(s) are installed in a vacuum container such as a vacuum electrotube (FIG. 16), vacuum bottle, vacuum chamber, and/or vacuum box with certain vacuum space. The vacuum container wall is made with a varieties of heat-conducting materials in combination of electric insulating materials that are selected from the group consisting of heat-conducting metals including stainless steels, aluminum, copper and metal alloys, vacuum-tube glass, vacuum lamp-bulb glass, electric insulating materials, carbon fibers composite materials, vinyl ester, epoxy, polyester resin, air-tight electric-insulating Kafuter 704 RTV silicone gel material, thermoplastic, highly heat-conductive graphene, graphite, cellulose nanofiber/epoxy resin nanocomposites, heat-conductive and electrical insulating plastics, heat-conductive and electrical insulating ceramics, heat-conductive and electrical insulating glass, fiberglass-reinforced plastic materials, borosilicate glass, Pyrex glass, fiberglass, sol-gel, silicone gel, silicone rubber, quartz mineral, diamond material, glass-ceramic, transparent ceramics, clear plastics, such as Acrylic (polymethyl methacrylate, PMMA), Butyrate (cellulose acetate butyrate), Lexan (polycarbonate), and PETG (glycol modified polyethylene terephthalate), polypropylene, polyethylene (or polyethene) and polyethylene HD, thermally conductive transparent plastics, heat conductive and electrical insulating paint, colorless glass, clear transparent plastics containing certain anti-reflection materials or coatings, clear glass containing certain anti-reflection materials or coatings and combinations thereof.

According to one of the various embodiments, the interfacing contact/seal between the container wall and the electrode plates and/or electric wires is made with heat-conductive and electrical insulating material(s). Depending on a given specific application and its associated temperature conditions, the interfacing contact/seal material(s) is selected from the group consisting of heat-conductive and electrical insulating plastics, epoxy, polyester resin, air-tight electric-insulating Kafuter 704 RTV silicone gel material, thermoplastic, heat-conductive and electrical insulating ceramics, heat-conductive and electrical insulating glass, highly heat-conductive graphene, graphite, clear plastics, for example, Acrylic (polymethyl methacrylate, PMMA), Butyrate (cellulose acetate butyrate), Lexan (polycarbonate), and PETG (glycol modified polyethylene terephthalate), polypropylene, polyethylene, and polyethylene HD, thermally conductive transparent plastics, heat conductive glues, electric insulating glues, heat conductive paint, electric insulating paint, heat conductive glass, borosilicate glass such as Pyrex glass, sol-gel, silicone gel, silicone rubber, quartz mineral, diamond material, cellulose nanofiber/epoxy resin nanocomposites, carbon fibers composite materials, glass-ceramic materials, transparent ceramics, clear transparent plastics containing anti-reflection materials and/or coating, clear glass containing anti-reflection materials or coatings and combinations thereof.

According to one of the various embodiments, an asymmetric function-gated isothermal electrons-based environmental heat energy utilization system comprises a low work function of Ag—O—Cs coated on an Ag metal electrode surface to serve as an emitter and a high work function of a Cu metallic conductor to serve as a collector in a vacuum condition.

According to one of the various embodiments, a prototype of an asymmetric function-gated isothermal electrons-based environmental heat energy utilization system comprises a pair of a low work function Ag—O—Cs film 1203 (coated on a silver electrode 1201 surface) and a high work function Mo metallic conductor 1202 separated by a vacuum space 1204 in a vacuum tube (FIG. 16). The Ag—O—Cs film 1203 coated on the silver electrode 1201 is used as the emitter while the Mo metallic conductor 1202 is used as the collector. In certain examples, a Mo—O—Cs film sometimes co-produced (during the Ag—O—Cs film making process) may also be used as the collector since it typically has a work function higher (bigger) than that of the Ag—O—Cs film. FIG. 16 illustrates an example of how such a prototype system can be fabricated and tested for isothermal electricity generation. In this example, a pair of silver and molybdenum electrodes was installed in a vacuum tube as shown in FIG. 16a. A cesium (Cs) vapor with a small amount of oxygen was introduced into the vacuum electrotube. During the fabrication process, the molybdenum electrode was used as a temporary anode to oxidize the silver electrode surface by a type of oxygen plasma discharge with the Cs vapor and subsequently resulted in the formation of an Ag—O—Cs film on the silver electrode 1201 surface as shown in FIG. 16b. Sometimes, this fabrication process also results in the co-generation of a Mo—O—Cs film on the molybdenum electrode 1202.

According to one of the various embodiments, a prototype of an asymmetric function-gated electrotube system like the one shown in FIG. 16b can isothermally generate electricity that can be measured at an ambient temperature such as 25° C. (298 K) using the input resistance of an electrometer as the load. It is predicted that when the outlet terminal 1206 of emitter 1201 is connected with a Model 237-ALG-2-type low-noise-cable positive (red) input connector of an electrometer while the output terminal 1207 of collector 1202 is connected with the negative (black) input connector, it will measure a positive electric current that is generated by the isothermal electricity generating system (FIG. 16b). When the asymmetric function-gated electrotube system and the electrometer are connected in the opposite (reverse) orientation in which the collector 1202 is connected to the positive (red) input connector of the electrometer while the emitter 1201 connected to the negative (black) input connector of the electrometer, the isothermal electricity generating system (FIG. 16b) is expected to give a measurable negative current to the electrometer.

These predicted features were successfully demonstrated in a preliminary experiment, where an asymmetric function-gated electrotube was placed into a Faraday shielding box made of metal foils and its isothermal electricity generation was measured with a Keithley 6514 system electrometer (Keithley Instruments, Inc., Cleveland, Ohio, USA). When the emitter 1201 was connected with the positive (red) input connector alligator clip of the Keithley 6514 system electrometer while the collector 1202 was connected with the negative (black) input connector alligator clip, a positive electrical current was indeed sensed by the Keithley 6514 electrometer. The steady-state electrical current density normal to the cross-section area of the interelectrode space was measured to be 5.17 pA/cm². Meanwhile, when the asymmetric function-gated electrotube system and electrometer were connected in the opposite (reverse) orientation, a negative electrical current with comparable amplitude was indeed measured through the Keithley 6514 electrometer. The steady-state electrical current density normal to the cross-section area of the interelectrode space measured in the reverse orientation was −4.50 pA/cm². The averaged steady-state electrical current density from the absolute values measured in the two orientations was 4.84±0.34 pA/cm².

Similarly, according to one of the various embodiments, it is predicted that when the emitter 1201 is connected with the positive (red) input connector alligator clip of a Keithley 6514 electrometer while the collector 1202 is connected with the negative (black) input connector alligator clip, it will measure a positive voltage that is generated by the isothermal electricity generating system (FIG. 16b). When the asymmetric function-gated electrotube system is connected with the electrometer in the opposite orientation, the isothermal electricity generating system (FIG. 16b) will give a measurable negative voltage to the electrometer. These predicted features were successfully demonstrated in the preliminary experiment as well. The steady-state output voltage averaged from the absolute values measured in the two orientations was about 140 mV in this example.

Based on the measured steady-state electrical current density (4.84±0.34 pA/cm²) and steady-state output voltage (about 140 mV), the isothermal electricity power generation density cross-section area of the interelectrode space was calculated to be about 6.78×10⁻¹³ Watt/cm² in this example of an experimental prototype system (FIG. 16b).

Table 7 presents more examples of experimental data on the isothermal electricity current density of the asymmetric work function-gated electrotubes similar to that of FIG. 16b as measured in both the normal and reverse orientations. It was noticed that the amplitude of the isothermal electricity current density measured in the normal orientation was occasionally somewhat larger than that measured in the opposite orientation. For each of the asymmetric work function-gated electrotube samples 1, 2, 3 and 4 listed in Table 7, the values of the isothermal electricity current density measured in the normal orientation were 5.17, 4.90, 7.06 and 9.62 pA/cm² which appeared to be slightly larger than the absolute values of those (−4.50, −1.63, −2.72, and −5.52 pA/cm²) in the reverse orientation. A similar trend was observed in the corresponding voltage measurements; the amplitude of isothermal electricity output voltage measured in the normal orientation was typically also somewhat larger than that measured in the reverse orientation. This might be explained by the interaction of an asymmetric work function-gated electrotube system with the Keithley 6514 electrometer. For example, if the input connector (black) of the Keithley 6514 system somehow gives a slightly positive voltage to the emitter when connected as in the reverse orientation, it could slightly push down the Fermi level at the emitter that could reduce the ability for the emitter to emit electrons which could explain the somewhat decreased isothermal electricity current density and consequently also the reduced voltage output.

As shown in Table 7, the isothermal electricity current density averaged from the absolute values measured in both orientations was 3.26, 4.87, and 7.57 pA/cm² for the asymmetric work function-gated electrotube samples 2, 3 and 4, respectively. The corresponding averaged voltage output was 94, 141 and 218 mV. The isothermal electricity power density calculated as the product of the isothermal electricity current density and corresponding voltage output was 3.07×10⁻¹³, 6.90×10⁻¹³, and 1.65×10⁻¹² Watt/cm² for the asymmetric work function-gated electrotube samples 2, 3 and 4, respectively, under the given experimental conditions without any optimization efforts. Therefore, these experimental data and the specific details were intended to show the proof of the principle according to one of the various embodiments and they shall not be viewed as a limit to its performance.

Table 7 lists more examples of experimental data on the isothermal electricity current density (pA/cm²) of asymmetric work function-gated electrotubes similar to that of FIG. 16b as measured in normal and reverse orientations and the observed output voltage (mV) and isothermal electricity power density (Watt/cm²).

| | Current | Current | | | Isothermal |
|---|---|---|---|---|---|
| | density | density | Averaged | Averaged | electricity |
| | (pA/cm²) in | (pA/cm²) | current | output | power |
| | normal | in reverse | density | voltage | density |
| | orientation | orientation | (pA/cm²) | (mV) | (Watt/cm²) |
| Electro- | 5.17 | −4.50 | 4.84 ± 0.34 | 140 | 6.78 × |
| tube | | | | | 10⁻¹³ |
| sample 1 | | | | | |
| Electro- | 4.90 ± 0.03 | −1.63 ± 0.07 | 3.26 ± 1.64 | 94 | 3.07 × |
| tube | | | | | 10⁻¹³ |
| sample 2 | | | | | |
| Electro- | 7.06 ± 0.15 | −2.72 ± 0.25 | 4.87 ± 2.19 | 141 | 6.90 × |
| tube | | | | | 10⁻¹³ |
| sample 3 | | | | | |
| Electro- | 9.62 ± 0.07 | −5.52 ± 0.03 | 7.57 ± 2.04 | 218 | 1.65 × |
| tube | | | | | 10⁻¹² |
| sample 4 | | | | | |

According to one of the various embodiments, the asymmetric function-gated thermal electron power generator system 1100 as illustrated in FIG. 14 operates isothermally where the temperature at the emitter (Tₑ) equals to that of the collector (Tₐ). Under the isothermal operating conditions (T=Tₑ=T_{c}), the ideal net flow density (flux) of the emitted electrons 1105 from the emitter 1101 to the collector 1102, which is defined also as the ideal isothermal electron flux (J_{isoT}) normal to the surfaces of the emitter and collector (also named as the ideal isothermal electricity current density defined as amps (A) per square centimeters of the cross-section area of the emitter-collector interelectrode space), can be calculated based on the Richardson-Dushman formulation using the following ideal isothermal current density (J_{isoT}) equation:

J_{isoT}=AT²(e^{-[WF(e)+e·V(e)]/kT}−e^{-[WF(c)+e·V(c)]/kT}) [11b]

Where A is the universal factor (as known as the Richardson-Dushman constant) can be expressed as

$$\frac{4 π {mek}^{2}}{{h}^{3}} \approx 120 Amp \text{/} ( {K}^{2} \cdot {cm}^{2} )$$

[where m is the electron mass, e is the electron unit charge, k is the Boltzmann constant and h is Planck constant]. T is the absolute temperature in Kelvin (K) for both the emitter and the collector; WF(e) is the work function of the emitter surface; the term of e·V (e) is the product of the electron unit charge e and the voltage V (e) at the emitter; k is the Boltzmann constant in (eV/K); WF(c) is the work function of the collector surface; and e·V (c) is the product of the electron charge e and the voltage V(c) at the collector.

Of particular significance is that the conversion of environmental thermal energy (latent heat) isothermally to electrical power without the need for an external energy-consuming heater or an exhaust, heat sink or the like, so that the energy efficiency is essentially 100% without being constrained by the second law of thermodynamics.

According to one of the various embodiments, when the voltage at the emitter (V(e)) is zero such as when the emitter is grounded as illustrated in FIG. 14c, the ideal net isothermal electrons flow density across the vacuum space from the emitter 1101 to the collector 1102 can be calculated using the following modified ideal isothermal current density (J_{isoT(gnd)}) equation:

J_{isoT(gnd)}=AT²(e^{-[WF(e)]/kT}−e^{-[WF(c)+e·V(c)]/kT}) [12]

According to one of the various embodiments, when the voltage at both the emitter (V(e)) and the collector (V(c)) are zero such as at the initial state of an isothermal electricity generation system 1100 as illustrated in FIG. 14a (or if/when the resistance of the circuit including the load 1108 and associated wire, electrodes and connection terminals 1106 and 1107, is zero), the maximum net isothermal electron flow density across the vacuum space from the emitter 1101 to the collector 1102 reaches the highest attainable, which is regarded as the “saturation” (upper limit) flux after the effects of any negative space charge and other limiting factors are all eliminated. This ideal saturation electron flux can be calculated using the following ideal saturation isothermal current density (J_{isoT(sat)}) equation:

J_{isoT(sat)}=AT²(e^{-[WF(e)]/kT}−e^{-[WF(c)+e·V(c)]/kT}) [13]

According to one of the various embodiments, the “open circuit” ideal saturation output voltage (Vₛₐₜ) at the equilibrium between the emitter and collector terminals (1106 and 1107) as shown in FIG. 14c can be expressed as the difference in the work functions:

$${V}_{sat} = \frac{{WF}_{( c )} - {WF}_{( e )}}{e} \qquad [ 14 ]$$

Where e is the electron charge which is 1 (an electron charge unit); and WF_{(c)} and WF₍ₑ₎ are the collector work function and the emitter work function, respectively, as illustrated in the 1100 (c) of FIG. 15 (right).

According to one of the various embodiments, the steady-state operating output voltage (Vₛₜ) between the emitter and collector terminals (1106 and 1107) can be expressed as:

Vₛₜ=V_{(c)}−V₍ₑ₎ [15]

Where V_{(c)} and V₍ₑ₎ are the steady-state operating voltages at the collector and emitter, respectively, as illustrated in the 1100 (b) FIG. 15 (middle).

According to one of the various embodiments, the ideal saturation electrical current (Iₛₐₜ) across the inter electrode space between the emitter and collector as shown in FIG. 14a can be expressed as the product of the interelectrode space cross section (emitter surface) area (S) and the ideal saturation isothermal electron flux as known as the saturation current density (J_{isoT(sat)}) with the following equation:

Iₛₐₜ=S·J_{isoT(sat)}=S·AT²(e^{-[WF(e)]/kT}−e^{-[WF(c)]/kT}) [16]

According to one of the various embodiments, the ideal steady-state operating electrical current (Iₛₜ) through the electrical load 1108 as shown in FIG. 14a can be expressed as:

$${I}_{st} = \frac{{R}_{l} + {R}_{m}}{{V}_{st}} \qquad [ 17 ]$$

Where R₁ is the resistance of the electrical load and Rₘ is any possible miscellaneous resistance from the circuit including the electrodes and wire materials; Vₛₜ is the steady-state operating output voltage as of Eq. [15].

According to one of the various embodiments, the effect of the asymmetric function-gated isothermal electricity generating activity is additive. That is, the asymmetric function-gated isothermal electricity generator systems like the one shown in FIG. 14 can be used in series and/or in parallel. When a plurality (n) of the asymmetric function-gated isothermal electricity generator systems like the one shown in FIG. 14 are used in the series, the total steady-state output voltage (Vₛₜ₍ₜₒₜₐₗ₎) is the summation of the steady-state output voltages (Vₛₜ₍ᵢ₎ as of Eq. [15]) from each of the asymmetric function-gated isothermal electricity generator systems:

Vₛₜ₍ₜₒₜₐₗ₎=Σᵢ₌₁ⁿVₛₜ₍ᵢ₎=Σᵢ₌₁ⁿ(V_{(c)i}−V₍ₑ₎ᵢ) [18]

Similarly, the total saturation output voltage (Vₛₐₜ₍ₜₒₜₐₗ₎) is the summation of the saturation output voltages (Vₛₐₜ₍ᵢ₎ as of Eq. [14]) from each of the asymmetric function-gated isothermal electricity generator systems operating in series:

$${V}_{sat ( total )} = {\sum }_{i = 1}^{n} {V}_{sat ( i )} = {\sum }_{i = 1}^{n} ( \frac{{WF}_{( c ) i} - {WF}_{( e ) i}}{e} ) \qquad [ 19 ]$$

According to one of the various embodiments, when pluralities (n) of the asymmetric function-gated isothermal electricity generator systems are used in the parallel, the total ideal electrical current (Iₛₐₜ₍ₜₒₜₐₗ₎) is the summation of the ideal electrical current (Iₛₐₜ₍ᵢ₎ as of Eq. [16]) from each of the asymmetric function-gated isothermal electricity generator systems:

$${I}_{sat ( total )} = {\sum }_{i = 1}^{n} {I}_{sat ( i )} \qquad [ 20 ]$$

Therefore, the asymmetric function-gated isothermal electricity production is additive. Pluralities (n) of the asymmetric function-gated isothermal electricity generator systems may be used in parallel and/or in series, depending on a given specific application and its associated operating conditions such as temperature conditions, and the properties of the barrier spaces such as their thickness and compositions, the properties of the emitter and collector electrodes and other physical chemistry properties.

When a plurality (n) of the asymmetric function-gated isothermal electricity generator systems operate in parallel, the total steady-state electrical current (Iₛₜ₍ₜₒₜₐₗ₎) is the summation of the steady-state electrical current (Iₛₜ₍ᵢ₎) from each of the asymmetric function-gated isothermal electricity generator systems while the total steady-state output voltage (Vₛₜ₍ₜₒₜₐₗ₎) remains the same.

When a plurality (n) of the asymmetric function-gated isothermal electricity generator systems operate in series, the total steady-state output voltage (Vₛₜ₍ₜₒₜₐₗ₎) is the summation of the steady-state output voltages (Vₛₜ₍ᵢ₎) from each of the asymmetric function-gated isothermal electricity generator systems while the total steady-state electrical current (Iₛₜ₍ₜₒₜₐₗ₎) remains the same.

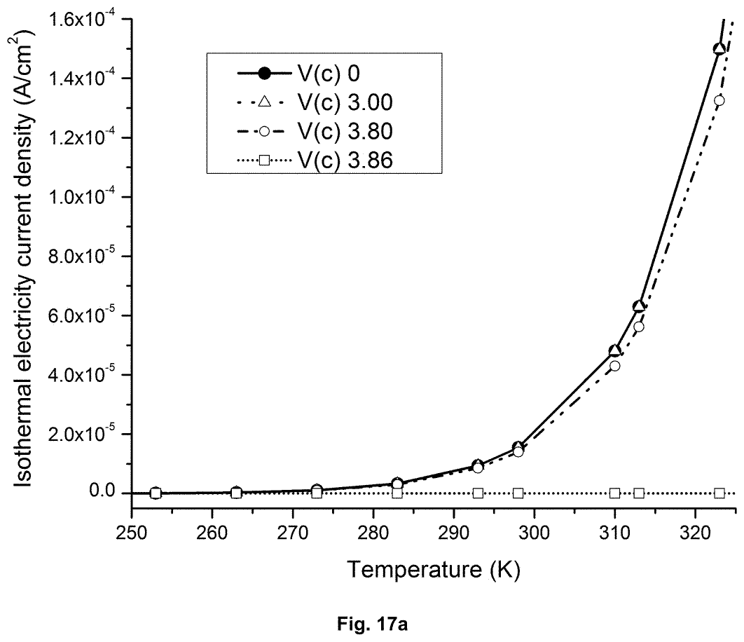

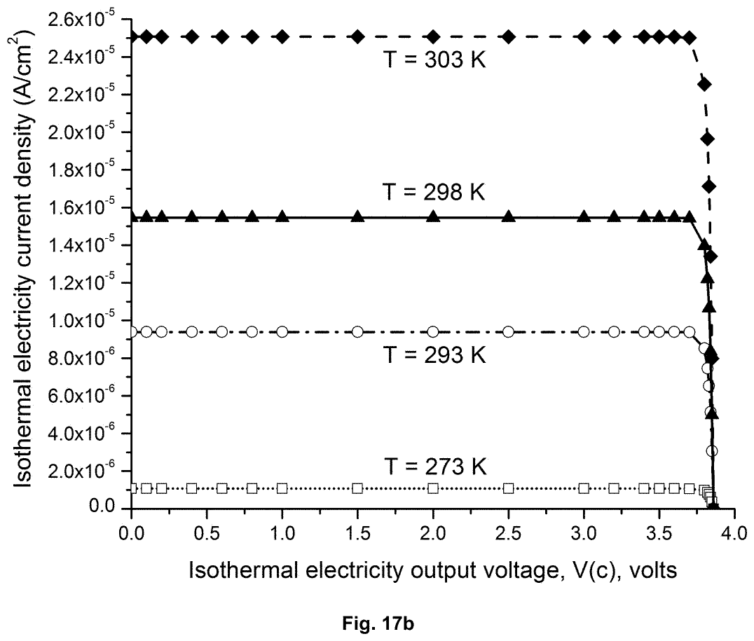

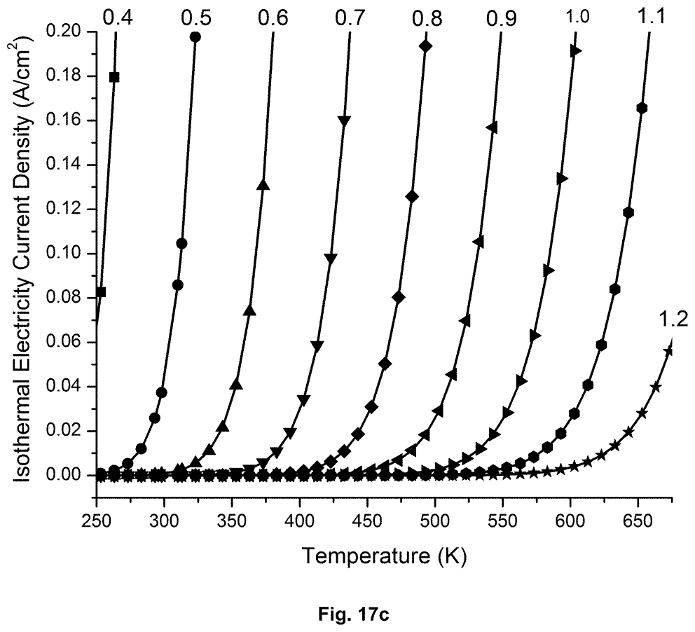

FIG. 17a presents the examples of the ideal isothermal electricity current density (A/cm² defined as amps (A) per square centimeters of the cross-section area of the emitter-collector interelectrode space) as a function of operating temperature T at various output voltage V(c) from 0.00 to 3.86 V, as calculated using Eq. 12 for a pair of emitter work function (WF(e)=0.70 eV) and collector work function (WF(c)=4.56 eV, copper Cu(110)), in which the emitter was grounded. Since the emitter was grounded, the output voltage equals to V(c), which is the difference between the collector voltage V(c) and the grounded emitter voltage (V(e)=0). Consequently, the isothermal electricity current density (A/cm²) with the output voltage V(c) of 0.00 V in the initial state as illustrated with energy diagram in the 1110(a) of FIG. 15 also represents the saturation isothermal current density as expressed in Eq. 13.

As shown in FIG. 17a, the ideal isothermal electricity current density curve with an output voltage V(c) of 3.00 V pretty much overlaps with that of the saturation isothermal current density (with V(c)=0.00 V) in a temperature (T) range from 225 K to 325 K. When the output voltage V(c) is raised to 3.80 V, the isothermal electricity current density curve lay only slightly below the maximum saturation isothermal current density curve. In these cases, the isothermal electricity current density increases dramatically as function of temperature T. However, when the output voltage V(c) is further raised to 3.86 V, the isothermal electricity current density is dramatically reduced to zero (a flat line), which represents the equilibrium state as shown in the 1110(c) of FIG. 15 (right) where the thermal electron flow from the emitter to the collector equals to that from the collector to the emitter, resulting in a net isothermal electricity current density of zero.

FIG. 17b presents the examples of the isothermal electricity current density (A/cm²) curves as a function of output voltage V(c) from 0.00 to 3.86 V at an operating environmental temperature of 273, 293, 298, and 303 K for a pair of emitter work function (WF(e)=0.70 eV) and collector work function (WF(c)=4.56 eV, copper Cu(110)) with the emitter grounded. These curves showed that the saturation isothermal electricity current density is pretty much constant (steady) in an output voltage V(c) range from 0.00 to 3.75 V at each of the operating environmental temperature of 273, 293, 298, and 303 K. Only when the output voltage V(c) is raised from 3.75 V to 3.86 V, the isothermal electricity current density is dramatically reduced to zero. At an output voltage in a range from 0 to 3.50V, the level of the steady-state isothermal electricity current density increases with temperature dramatically from the level of 1.07 μA/cm² at 273 K (0° C.) to the levels of 9.39, 15.5, and 25.1 μA/cm² at 293 K (20° C.), 298 K (25° C.), and 303 K (30° C.), respectfully.

Table 8 lists the ideal isothermal electricity current density (A/cm²) values as a function of operating temperature T in a range from 203 K (−70° C.) to 673 K (400° C.) at a number of output voltage V(c) values including 0.00, 1.50, 3.00, 3.50, 3.80 and 3.86 V, as calculated using Eq. 12 for a pair of emitter work function (WF(e)=0.70 eV) and collector work function (WF(c)=4.56 eV, copper Cu(110)) where the emitter was grounded. The data showed that, with a reasonable output voltage V(c) of about 3 V, the isothermal electricity current density is strongly dependent on temperature T in a range from 2.07×10⁻¹¹ (A/cm²) at 203 K (−70° C.) to 1.55×10⁻⁵ (A/cm²) at 298K (25° C.), and to as much as 311 (A/cm²) at 673 K (400° C.).

Table 8 presents the examples of the ideal isothermal electricity current density (A/cm²) as a function of operating temperature T at various output voltage V(c) from 0.00 to 3.86 V, calculated using Eq. 12 for a pair of emitter work function (WF(e)=0.70 eV) and collector work function (WF(c)=4.56 eV, copper Cu(110)). The emitter was grounded and the output voltage V(c) is the voltage difference between the collector and the grounded emitter.

| T (K) | V(c) 0.00 | V(c) 1.50 | V(c) 3.00 | V(c) 3.50 | V(c) 3.80 | V(c) 3.86 |
|---|---|---|---|---|---|---|
| 203 | 2.07E−11 | 2.07E−11 | 2.07E−11 | 2.07E−11 | 2.00E−11 | 0 |
| 213 | 1.49E−10 | 1.49E−10 | 1.49E−10 | 1.49E−10 | 1.43E−10 | 0 |
| 223 | 9.04E−10 | 9.04E−10 | 9.04E−10 | 9.04E−10 | 8.64E−10 | 0 |
| 233 | 4.71E−09 | 4.71E−09 | 4.71E−09 | 4.71E−09 | 4.47E−09 | 0 |
| 243 | 2.15E−08 | 2.15E−08 | 2.15E−08 | 2.15E−08 | 2.03E−08 | 0 |
| 253 | 8.74E−08 | 8.74E−08 | 8.74E−08 | 8.74E−08 | 8.18E−08 | 0 |
| 263 | 3.20E−07 | 3.20E−07 | 3.20E−07 | 3.20E−07 | 2.97E−07 | 0 |
| 273 | 1.07E−06 | 1.07E−06 | 1.07E−06 | 1.07E−06 | 9.86E−07 | 0 |
| 283 | 3.29E−06 | 3.29E−06 | 3.29E−06 | 3.29E−06 | 3.01E−06 | 0 |
| 293 | 9.39E−06 | 9.39E−06 | 9.39E−06 | 9.39E−06 | 8.52E−06 | 0 |
| 298 | 1.55E−05 | 1.55E−05 | 1.55E−05 | 1.55E−05 | 1.40E−05 | 0 |
| 310 | 4.81E−05 | 4.81E−05 | 4.81E−05 | 4.81E−05 | 4.30E−05 | 0 |
| 313 | 6.30E−05 | 6.30E−05 | 6.30E−05 | 6.30E−05 | 5.62E−05 | 0 |
| 323 | 1.50E−04 | 1.50E−04 | 1.50E−04 | 1.50E−04 | 1.32E−04 | 0 |
| 333 | 3.39E−04 | 3.39E−04 | 3.39E−04 | 3.39E−04 | 2.97E−04 | 0 |
| 343 | 7.32E−04 | 7.32E−04 | 7.32E−04 | 7.32E−04 | 6.36E−04 | 0 |
| 353 | 0.00152 | 0.00152 | 0.00152 | 0.00152 | 0.00131 | 0 |
| 363 | 0.00302 | 0.00302 | 0.00302 | 0.00302 | 0.00258 | 0 |
| 373 | 0.00582 | 0.00582 | 0.00582 | 0.00582 | 0.00492 | 0 |
| 383 | 0.01083 | 0.01083 | 0.01083 | 0.01083 | 0.00907 | 0 |
| 393 | 0.01956 | 0.01956 | 0.01956 | 0.01956 | 0.01623 | 0 |
| 403 | 0.03435 | 0.03435 | 0.03435 | 0.03435 | 0.02824 | 0 |
| 413 | 0.05877 | 0.05877 | 0.05877 | 0.05877 | 0.04788 | 0 |
| 423 | 0.09814 | 0.09814 | 0.09814 | 0.09814 | 0.07922 | 0 |
| 433 | 0.16024 | 0.16024 | 0.16024 | 0.16023 | 0.12815 | 0 |
| 443 | 0.25616 | 0.25616 | 0.25616 | 0.25614 | 0.20296 | 0 |
| 453 | 0.40151 | 0.40151 | 0.40151 | 0.40147 | 0.31518 | 0 |
| 463 | 0.61782 | 0.61782 | 0.61782 | 0.61775 | 0.48049 | 0 |
| 473 | 0.93436 | 0.93436 | 0.93436 | 0.93422 | 0.71996 | 0 |
| 483 | 1.39028 | 1.39028 | 1.39028 | 1.39004 | 1.0614 | 0 |
| 493 | 2.03731 | 2.03731 | 2.03731 | 2.03688 | 1.54106 | 0 |
| 503 | 2.94281 | 2.94281 | 2.94281 | 2.94208 | 2.20558 | 0 |
| 513 | 4.1935 | 4.1935 | 4.1935 | 4.19229 | 3.11423 | 0 |
| 523 | 5.89976 | 5.89976 | 5.89976 | 5.89775 | 4.34142 | 0 |
| 533 | 8.20048 | 8.20048 | 8.20048 | 8.19725 | 5.97966 | 0 |
| 543 | 11.2688 | 11.2688 | 11.2688 | 11.26367 | 8.14272 | 0 |
| 553 | 15.31839 | 15.31839 | 15.31839 | 15.31037 | 10.96923 | 0 |
| 563 | 20.61061 | 20.61061 | 20.61061 | 20.59826 | 14.62655 | 0 |
| 573 | 27.46246 | 27.46246 | 27.46246 | 27.44373 | 19.31508 | 0 |
| 583 | 36.25534 | 36.25534 | 36.25534 | 36.22732 | 25.27281 | 0 |
| 593 | 47.44464 | 47.44464 | 47.44464 | 47.40327 | 32.78025 | 0 |
| 603 | 61.57024 | 61.57024 | 61.57023 | 61.5099 | 42.16566 | 0 |
| 613 | 79.26778 | 79.26778 | 79.26778 | 79.18081 | 53.81059 | 0 |
| 623 | 101.2809 | 101.2809 | 101.28089 | 101.15693 | 68.15564 | 0 |
| 633 | 128.47419 | 128.47419 | 128.47418 | 128.29937 | 85.70654 | 0 |
| 643 | 161.84712 | 161.84712 | 161.84709 | 161.60307 | 107.04041 | 0 |
| 653 | 202.54861 | 202.54861 | 202.54856 | 202.21123 | 132.81218 | 0 |
| 663 | 251.89254 | 251.89254 | 251.89246 | 251.43047 | 163.76124 | 0 |
| 673 | 311.37387 | 311.37387 | 311.37375 | 310.74663 | 200.71813 | 0 |

According to one of the various embodiments, when the emitter is grounded, the ideal isothermal electricity power production density (W/cm²) at various output voltage V(c) volts can be expressed as:

P_{isoT(gnd)}=AT²(e^{-[WF(e)]/kT}−e^{[WF(c)+e·V(c)]/kT})V(c) [21]

Table 9 list the ideal isothermal electricity power production density defined as Watt (W) per square centimeters (W/cm²) as a function of operating temperature T in a range from 203 K (−70° C.) to 673 K (400° C.) at a number of output voltage V(c) values including 0.00, 1.50, 3.00, 3.50, 3.80 and 3.86 V, as calculated using Eq. 21 for a pair of emitter work function (WF(e)=0.70 eV) and collector work function (WF(c)=4.56 eV, copper Cu(110)) where the emitter was grounded. The data showed that the output voltage V(c) that gave the best isothermal electricity power production density (W/cm²) was about 3.50 V in this example. The isothermal power production density (W/cm²) at output voltage V(c) of 3.50 V is strongly dependent on temperature T, which is in a range from 7.24×10⁻¹¹ (W/cm²) at 203 K (−70° C.) to 5.41×10⁻⁵ (W/cm²) at 298K (25° C.), and to as much as 1090 (W/cm²) at 673 K (400° C.).

Table 9 presents the examples of the ideal isothermal electricity power production density defined as Watt (W) per square centimeters (W/cm²) as a function of operating temperature T at various output voltage V(c) from 0.00 to 3.86 V, calculated using Eq. 21 for a pair of emitter work function (WF(e)=0.70 eV) and collector work function (WF(c)=4.56 eV, copper Cu(110)) where the emitter is grounded.

| T | V(c) | V(c) | V(c) | V(c) | V(c) | V(c) |
|---|---|---|---|---|---|---|
| (K) | 0.00 | 1.50 | 3.00 | 3.50 | 3.80 | 3.86 |
| 203 | 0 | 3.10E−11 | 6.21E−11 | 7.24E−11 | 7.61E−11 | 0 |
| 213 | 0 | 2.24E−10 | 4.47E−10 | 5.22E−10 | 5.45E−10 | 0 |
| 223 | 0 | 1.36E−09 | 2.71E−09 | 3.16E−09 | 3.28E−09 | 0 |
| 233 | 0 | 7.07E−09 | 1.41E−08 | 1.65E−08 | 1.70E−08 | 0 |
| 243 | 0 | 3.23E−08 | 6.45E−08 | 7.53E−08 | 7.71E−08 | 0 |
| 253 | 0 | 1.31E−07 | 2.62E−07 | 3.06E−07 | 3.11E−07 | 0 |
| 263 | 0 | 4.80E−07 | 9.60E−07 | 1.12E−06 | 1.13E−06 | 0 |
| 273 | 0 | 1.60E−06 | 3.21E−06 | 3.74E−06 | 3.75E−06 | 0 |
| 283 | 0 | 4.93E−06 | 9.86E−06 | 1.15E−05 | 1.14E−05 | 0 |
| 293 | 0 | 1.41E−05 | 2.82E−05 | 3.29E−05 | 3.24E−05 | 0 |
| 298 | 0 | 2.32E−05 | 4.64E−05 | 5.41E−05 | 5.31E−05 | 0 |
| 310 | 0 | 7.21E−05 | 1.44E−04 | 1.68E−04 | 1.63E−04 | 0 |
| 313 | 0 | 9.45E−05 | 1.89E−04 | 2.20E−04 | 2.13E−04 | 0 |
| 323 | 0 | 2.25E−04 | 4.49E−04 | 5.24E−04 | 5.03E−04 | 0 |
| 333 | 0 | 5.08E−04 | 1.02E−03 | 1.19E−03 | 1.13E−03 | 0 |
| 343 | 0 | 1.10E−03 | 2.20E−03 | 2.56E−03 | 2.42E−03 | 0 |
| 353 | 0 | 2.28E−03 | 4.56E−03 | 5.32E−03 | 4.98E−03 | 0 |
| 363 | 0 | 4.53E−03 | 9.06E−03 | 1.06E−02 | 9.80E−03 | 0 |
| 373 | 0 | 8.73E−03 | 1.75E−02 | 2.04E−02 | 1.87E−02 | 0 |
| 383 | 0 | 1.62E−02 | 3.25E−02 | 3.79E−02 | 3.45E−02 | 0 |
| 393 | 0 | 2.93E−02 | 5.87E−02 | 6.85E−02 | 6.17E−02 | 0 |
| 403 | 0 | 5.15E−02 | 1.03E−01 | 1.20E−01 | 1.07E−01 | 0 |
| 413 | 0 | 8.82E−02 | 1.76E−01 | 2.06E−01 | 1.82E−01 | 0 |
| 423 | 0 | 1.47E−01 | 2.94E−01 | 3.43E−01 | 3.01E−01 | 0 |
| 433 | 0 | 2.40E−01 | 4.81E−01 | 5.61E−01 | 4.87E−01 | 0 |
| 443 | 0 | 3.84E−01 | 7.68E−01 | 8.96E−01 | 7.71E−01 | 0 |
| 453 | 0 | 6.02E−01 | 1.20E+00 | 1.41E+00 | 1.20E+00 | 0 |
| 463 | 0 | 9.27E−01 | 1.85E+00 | 2.16E+00 | 1.83E+00 | 0 |
| 473 | 0 | 1.40E+00 | 2.80E+00 | 3.27E+00 | 2.74E+00 | 0 |
| 483 | 0 | 2.09E+00 | 4.17E+00 | 4.87E+00 | 4.03E+00 | 0 |
| 493 | 0 | 3.06E+00 | 6.11E+00 | 7.13E+00 | 5.86E+00 | 0 |
| 503 | 0 | 4.41E+00 | 8.83E+00 | 1.03E+01 | 8.38E+00 | 0 |
| 513 | 0 | 6.29E+00 | 1.26E+01 | 1.47E+01 | 1.18E+01 | 0 |
| 523 | 0 | 8.85E+00 | 1.77E+01 | 2.06E+01 | 1.65E+01 | 0 |
| 533 | 0 | 1.23E+01 | 2.46E+01 | 2.87E+01 | 2.27E+01 | 0 |
| 543 | 0 | 1.69E+01 | 3.38E+01 | 3.94E+01 | 3.09E+01 | 0 |
| 553 | 0 | 2.30E+01 | 4.60E+01 | 5.36E+01 | 4.17E+01 | 0 |
| 563 | 0 | 3.09E+01 | 6.18E+01 | 7.21E+01 | 5.56E+01 | 0 |
| 573 | 0 | 4.12E+01 | 8.24E+01 | 9.61E+01 | 7.34E+01 | 0 |
| 583 | 0 | 5.44E+01 | 1.09E+02 | 1.27E+02 | 9.60E+01 | 0 |
| 593 | 0 | 7.12E+01 | 1.42E+02 | 1.66E+02 | 1.25E+02 | 0 |
| 603 | 0 | 9.24E+01 | 1.85E+02 | 2.15E+02 | 1.60E+02 | 0 |
| 613 | 0 | 1.19E+02 | 2.38E+02 | 2.77E+02 | 2.04E+02 | 0 |
| 623 | 0 | 1.52E+02 | 3.04E+02 | 3.54E+02 | 2.59E+02 | 0 |
| 633 | 0 | 1.93E+02 | 3.85E+02 | 4.49E+02 | 3.26E+02 | 0 |
| 643 | 0 | 2.43E+02 | 4.86E+02 | 5.66E+02 | 4.07E+02 | 0 |
| 653 | 0 | 3.04E+02 | 6.08E+02 | 7.08E+02 | 5.05E+02 | 0 |
| 663 | 0 | 3.78E+02 | 7.56E+02 | 8.80E+02 | 6.22E+02 | 0 |
| 673 | 0 | 4.67E+02 | 9.34E+02 | 1.09E+03 | 7.63E+02 | 0 |

FIG. 17c presents the examples of the ideal isothermal electricity current density (A/cm²) at an output voltage V(c) of 3.00 V as a function of operating environmental temperature T with a series of emitter work function (WF(e)) values including 0.4, 0.5, 0.6, 0.7, 0.8, 0.9, 1.0, 1.1 or 1.2 eV in pairing with the collector work function (WF(c)=4.56 eV, copper Cu(110)) with the emitter grounded. The data showed that use of emitter with a lower work function is highly imperative to utilize environmental heat to generate isothermal electricity. Therefore, according to one of various embodiments, it is a preferred practice to employ emitter with a low work function that is selected from the group consisting of 0.3, 0.4, 0.5, 0.6, 0.7, 0.8, 0.9, 1.0, 1.1 and 1.2 eV and/or within a range bounded by any two of these values for isothermal electricity generation in a temperature range from 250 K to 673 K.

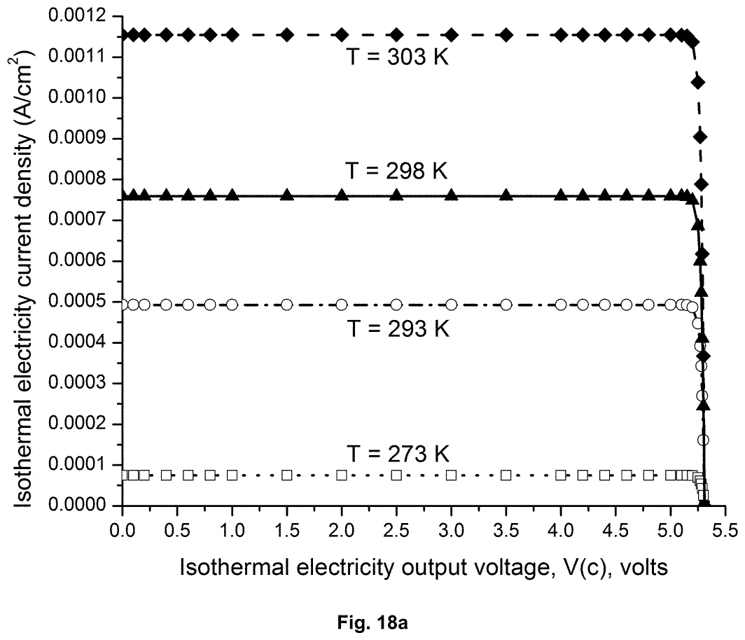

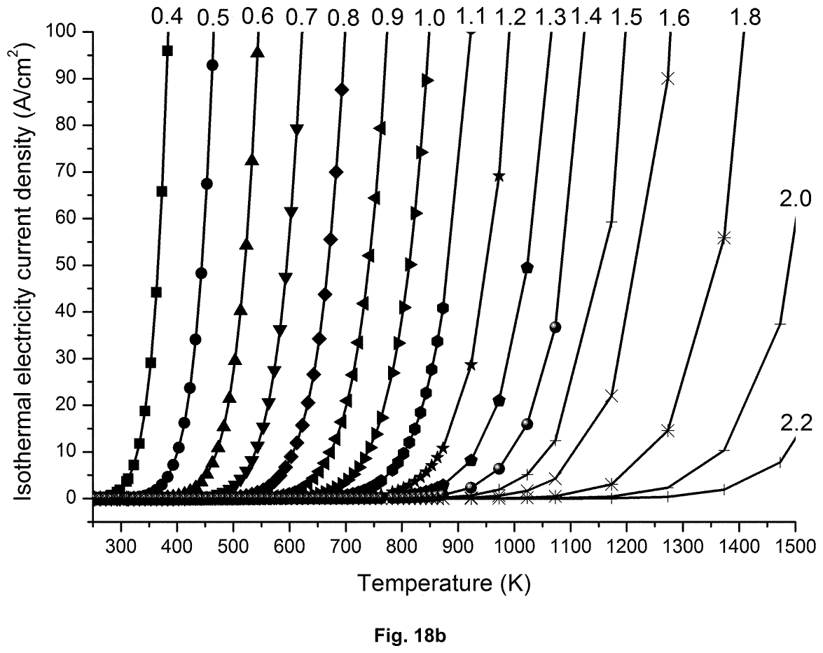

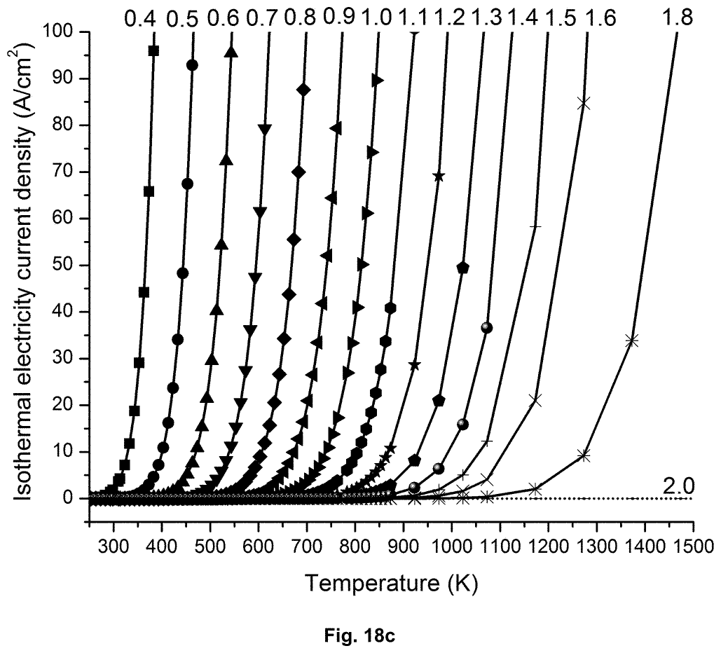

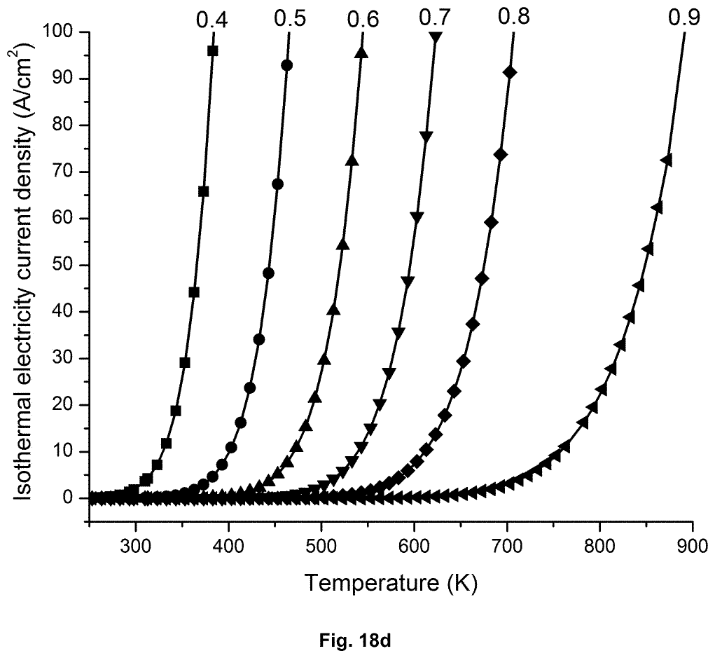

FIG. 18a presents the examples of the ideal isothermal electricity current density (A/cm²) curves as a function of output voltage V(c) volts in a range from 0.00 to 5.31 V at an operating environmental temperature of 273, 293, 298, and 303 K for a pair of emitter work function (WF(e)=0.60 eV) and collector work function (WF(c)=5.91 eV, platinum Pt(111)) with the emitter grounded. These curves showed that the isothermal electricity current density is pretty much constant (steady) in an output voltage V(c) range from 0.00 to 5.00 V at each of the operating environmental temperature of 273, 293, 298, and 303 K. Only when the output voltage V(c) is raised beyond 5.0 V up to the limit of 5.31 V, the isothermal electricity current density is dramatically reduced to zero. The level of the steady-state isothermal electricity current density at an output voltage of 5.00V increases dramatically with temperature from 7.50×10⁻⁵ A/cm² at 273 K (0° C.) to 4.93×10⁻⁴ A/cm² at 293 K (20° C.), 7.59×10⁻⁴ A/cm² at 298 K (25° C.), and to 1.15×10⁻³ A/cm² at 303 K (30° C.).

FIG. 18b presents the examples of the ideal isothermal electricity current density (A/cm²) as a function of operating environmental temperature T with a series of emitter work function (WF(e)) values including 0.4, 0.5, 0.6, 0.7, 0.8, 0.9, 1.0, 1.1, 1.2, 1.3, 1.4, 1.5, 1.6, 1.8, 2.0, and 2.2 eV, each in pair with the collector work function (WF(c)=5.91 eV, platinum Pt(111)) with the emitter grounded. The data showed that it is a preferred practice to use emitter with a lower work function to utilize environmental heat to generate isothermal electricity. Therefore, according to one of various embodiments, it is a preferred practice to employ emitter with a low work function that is selected from the group consisting of 0.3, 0.4, 0.5, 0.6, 0.7, 0.8, 0.9, 1.0, 1.1, 1.2, 1.3, 1.4, 1.5, 1.6, 1.8, 2.0, and 2.2 eV and/or within a range bounded by any two of these values for isothermal electricity generation in a temperature range from 250 to 1500 K.

FIG. 18c presents the examples of the ideal isothermal electricity current density (A/cm²) at an output voltage V(c) of 4.00 V as a function of operating environmental temperature T with a series of emitter work function (WF(e)) values including 0.4, 0.5, 0.6, 0.7, 0.8, 0.9, 1.0, 1.1, 1.2, 1.3, 1.4, 1.5, 1.6, 1.8, and 2.0 eV, each in pair with the collector work function (WF(c)=5.91 eV, platinum Pt(111)) with the emitter grounded. The data showed that it is a preferred practice to use emitter with a lower work function to utilize environmental heat to generate isothermal electricity. Therefore, according to one of various embodiments, it is a more preferred practice to employ emitter with a low work function that is selected from the group consisting of 0.3, 0.4, 0.5, 0.6, 0.7, 0.8, 0.9, 1.0, 1.1, 1.2, 1.3, 1.4, 1.5, 1.6, and 1.8 eV and/or within a range bounded by any two of these values for isothermal electricity generation with an output voltage V(c) of 4.00 V in a temperature range from 250 to 1500 K.

FIG. 18d presents the examples of the ideal isothermal electricity current density (A/cm²) at an output voltage V(c) of 5.00 V as a function of operating environmental temperature T with a series of emitter work function (WF(e)) values including 0.4, 0.5, 0.6, 0.7, 0.8, and 0.9 eV, each in pair with the collector work function (WF(c)=5.91 eV, platinum Pt(111)) with the emitter grounded. The data showed that it is a preferred practice to use emitter with a lower work function to utilize environmental heat to generate isothermal electricity. Therefore, according to one of various embodiments, it is a preferred practice to employ emitter with a low work function that is selected from the group consisting of 0.3, 0.4, 0.5, 0.6, 0.7, 0.8, and 0.9 eV and/or within a range bounded by any two of these values for isothermal electricity generation with an output voltage V(c) of 5.00 V in a temperature range from 250 to 900 K.

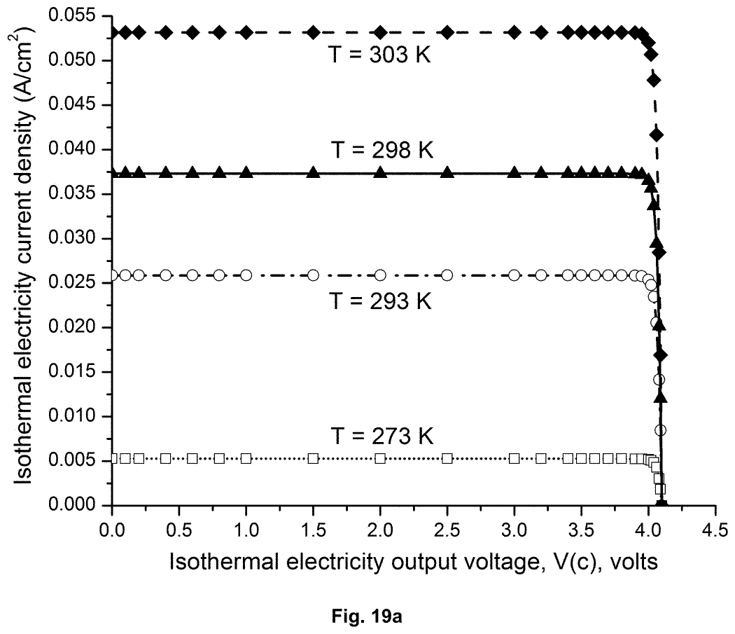

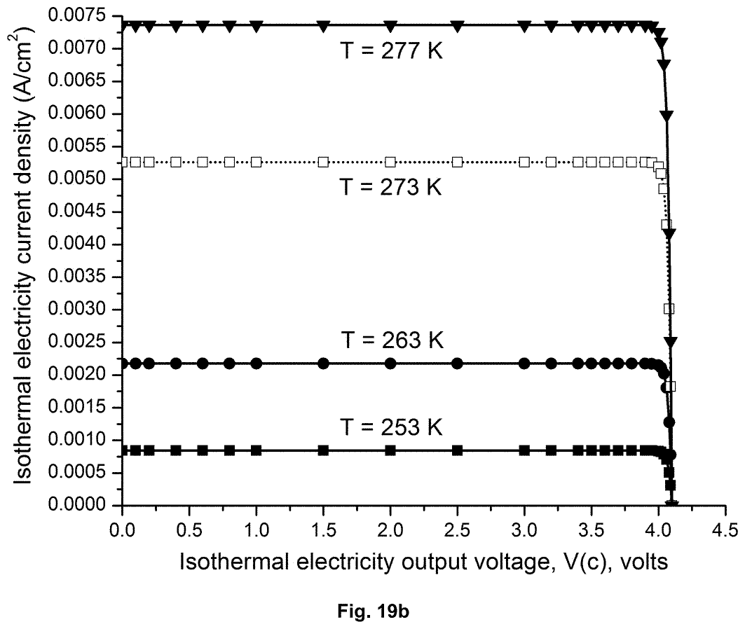

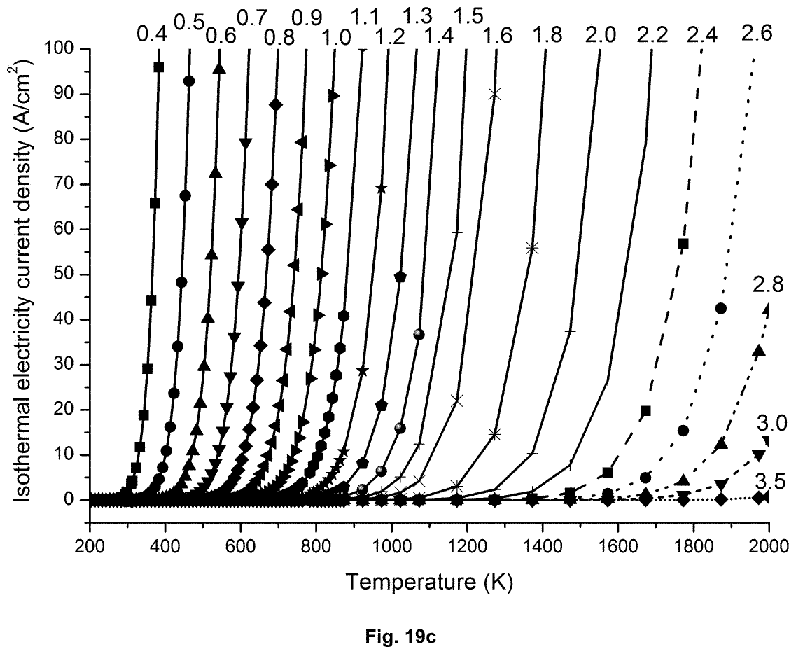

FIG. 19a presents the examples of the ideal isothermal electricity current density (A/cm²) curves as a function of output voltage V(c) from 0.00 to 4.10 V at operating environmental temperature of 273, 293, 298, and 303 K for a pair of emitter work function (WF(e)=0.50 eV) and collector work function (WF(c)=4.60 eV, graphene and/or graphite) with the emitter grounded. These curves showed that the isothermal electricity current density is pretty much constant (steady) in a range of output voltage V(c) from 0.00 to 4.00 V at each of the operating environmental temperature of 273, 293, 298, and 303 K. Only when the output voltage V(c) is raised beyond 4.00 V up to the limit of 4.10 V, the isothermal electricity current density is dramatically reduced to zero. The level of the steady-state isothermal electricity current density at an output voltage of 3.50 V increases dramatically with temperature from 5.26×10⁻³ A/cm² at 273 K (0° C.) to 2.59×10⁻² A/cm² at 293 K (20° C.), 3.73×10⁻² A/cm² at 298 K (25° C.), and to 5.32×10⁻² A/cm² at 303 K (30° C.).

FIG. 19b presents the examples of the ideal isothermal electricity current density (A/cm²) curves as a function of output voltage V(c) volts from 0.00 to 4.10 V at a freezing and/or refrigerating temperature of 253, 263, 273, and 277 K for a pair of emitter work function (WF(e)=0.50 eV) and collector work function (WF(c)=4.60 eV, graphene and/or graphite) with the emitter grounded. These curves showed that the isothermal electricity current density is pretty much constant in a range of output voltage V(c) from 0.00 to 4.00 V at each of the operating temperature of 253, 263, 273, and 277 K. Only when the output voltage V(c) is raised beyond 4.00 V up to the limit of 4.10 V, the isothermal electricity current density is dramatically reduced to zero. The saturation level of the steady-state isothermal electricity current density at an output voltage of 3.50 V increases dramatically with temperature from 8.42×10⁻⁴ A/cm² at 253 K (−20° C.) to 2.18×10⁻³ A/cm² at 263 K (−10° C.), to 5.26×10⁻³ A/cm² at 273 K (0° C.) and to 7.36×10⁻³ A/cm² at 277 K (4° C.).

FIG. 19c presents the examples of the ideal isothermal electricity current density (A/cm²) as a function of operating environmental temperature T with a series of emitter work function (WF(e)) values including 0.4, 0.5, 0.6, 0.7, 0.8, 0.9, 1.0, 1.1, 1.2, 1.3, 1.4, 1.5, 1.6, 1.8, 2.0, 2.2, 2.4, 2.6, 2.8, 3.0, and 3.5 eV, each in pair with a collector work function (WF(c)=4.60 eV, graphene and/or graphite) with the emitter grounded. The data showed that it is a preferred practice to use an emitter with a lower work function to utilize environmental heat to generate isothermal electricity. Therefore, according to one of various embodiments, it is a preferred practice to employ an emitter with a low work function that is selected from the group consisting of 0.3, 0.4, 0.5, 0.6, 0.7, 0.8, 0.9, 1.0, 1.1, 1.2, 1.3, 1.4, 1.5, 1.6, 1.8, 2.2, 2.4, 2.6, 2.8, and 3.0 eV and/or within a range bounded by any two of these values for isothermal electricity generation in a temperature range from 200 to 2000 K.

FIG. 20 presents an example of an integrated isothermal electricity generator system 1300 that comprises multiple pairs of emitters and collectors working in series. As illustrated in FIG. 20, the system 1300 comprises four parallel electric conductor plates 1301, 1302, 1321 and 1332 set apart with barrier spaces (such as vacuum spaces) 1304, 1324, and 1334 in between the conductor plates. Accordingly, the first electric conductive plate 1301 has its right side surface coated with a thin layer of low work function (LWF) film 1303 serving as the first emitter; The second electric conductive plate 1302 has its left side surface coated with a thin layer of high work function (HWF) film 1309 serving as the first collector while its right side surface coated with a thin layer of low work function (LWF) film 1323 serving as the second emitter; The third electric conductive plate 1321 has its left side surface coated with a thin layer of high work function (HWF) film 1329 serving as the second collector while its right side surface coated with a thin layer of low work function (LWF) film 1333 serving as the third emitter; The fourth electric conductive plate 1332 has its left side surface coated with a thin layer of high work function (HWF) film 1339 serving as third (terminal) collector; The first barrier space 1304 allows the thermal electron flow 1305 to pass through ballistically between the first pair of emitter 1303 and collector 1309; The second barrier space 1324 allows the thermal electron flow 1325 to pass through ballistically between the second pair of emitter 1323 and collector 1329; The third barrier space 1334 allows the thermal electron flow 1335 to pass through ballistically between the third pair of emitter 1333 and collector 1339.

According to one of the various embodiments, it is a preferred practice to employ: a first capacitor 1361 connected in between the first and second electric conductor plates 1301 and 1302; a second capacitor 1362 linked in between the second and third conductor plates 1302 and 1321; a third capacitor 1363 used in between third and the fourth conductor plates 1321 and 1332 as illustrated in FIG. 20. The use of capacitors in this manner can typically provide better system stability and robust isothermal electricity delivery. In this example with the first conductor plate 1301 grounded, isothermal electricity can be delivered through outlet terminals 1306 and 1376 or 1377 depending on the specific output power needs. When the isothermal electricity is delivered through outlet terminals 1306 and 1376 across a pair of emitter and collector, the steady-state operating output voltage equals to V(c), which typically can be around 3-4 V depending on the system operating conditions including the load resistance and the difference in work function between the emitter and the collector. When the isothermal electricity is delivered through outlet terminals 1306 and 1377 across three pairs of emitters and collectors, the steady-state operating output voltage is 3×Vc, which typically can be about 9-12 V in this example.

According to one of the various embodiments, the isothermal electricity of the 1300 system (FIG. 20) can be delivered also through outlet terminals 1376 and 1377. In this case, the V(c) voltage at the second electric conductor plate 1302 generated by the activity of the first emitter (conductor 1301 with LWF film 1303) and first collector (HWF plate 1309) may serve as a bias voltage for the second emitter (LWF film 1323 on the right side surface of the second electric conductor plate 1302) so that the second emitter 1323 will more readily emit thermal electrons towards the second collector 1329 on the left side surface of the third conductor plate 1321 which has the third emitter 1333. Subsequently, the V(c) created at the second collector 1329 of the third conductor plate 1321 can serve as a bias voltage for the third emitter 1333 to more readily emit thermal electrons towards the terminal collector 1339 at the fourth conductor plate 1332 to facilitate the generation of isothermal electricity for delivery through the outlet terminals 1376 and 1377. Therefore, use of this special feature can help better extract environmental energy especially when the operating environmental temperature is relatively low or when the work function of certain emitters alone may not be entirely low enough to function effectively. When the isothermal electricity is delivered through the outlet terminals 1376 and 1377, the steady-state operating output voltage is 2×Vc, which typically can be about 6-8 V in this case.

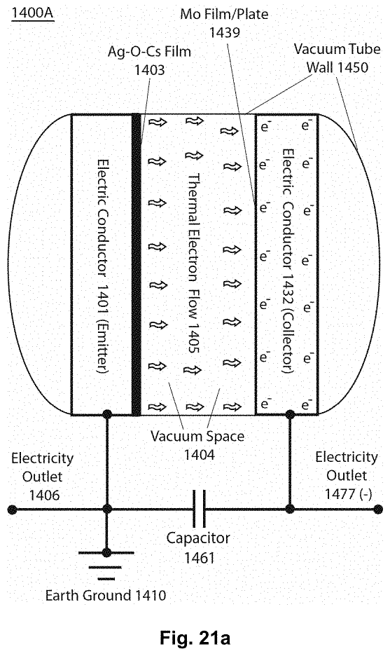

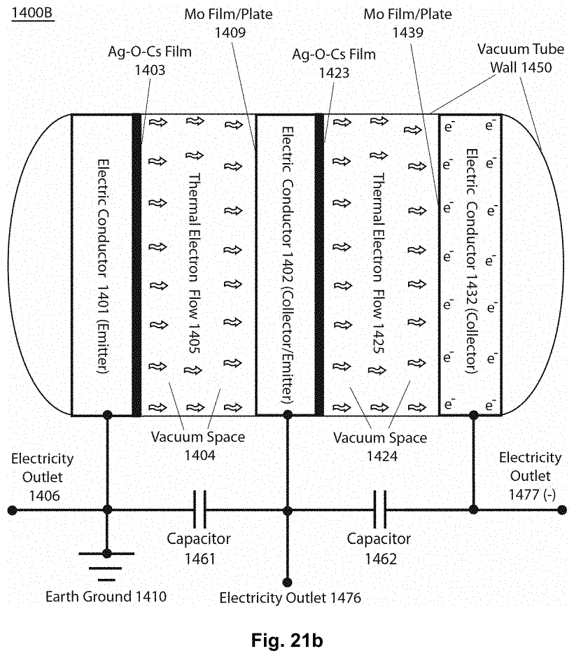

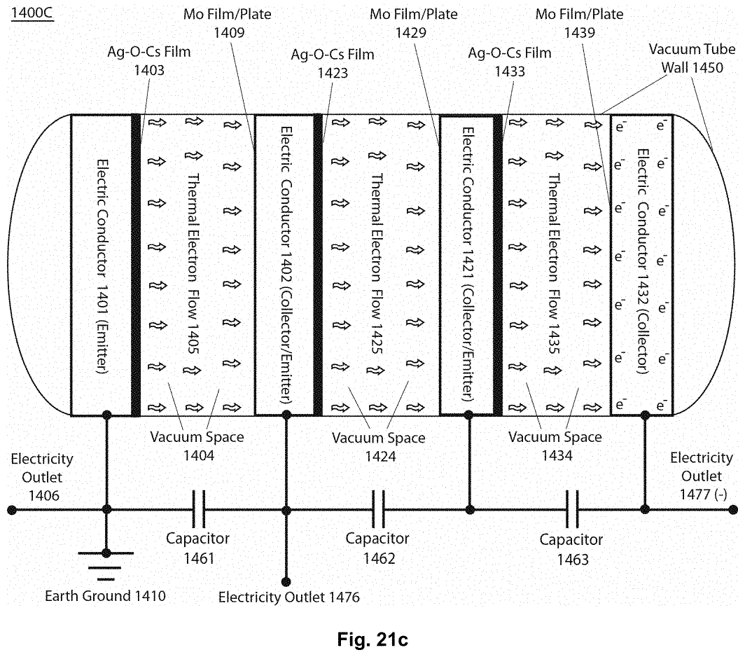

FIG. 21a presents an example of a prototype for an isothermal electricity generator system 1400A that has a pair of emitter (work function 0.7 eV) and collector (work function 4.36 eV) installed in a vacuum tube chamber. As illustrated in FIG. 21a, the system 1400A comprises a thin layer of low work function Ag—O—Cs film 1403 coated on the right side surface of electric conductor plate 1401 to serve as the emitter, a vacuum space 1404 allowing the thermal electron flow 1405 to pass through ballistically between the emitter and collector, a high work function Mo film/plate 1439 coated on the left side surface of the second electric conductor plate 1432 facing the emitter 1403 to serve as the collector, a vacuum tube wall 1450 that is in contact with the edges of the electric conductor plates 1401 and 1432 to allow environmental heat to transfer between the tube wall and the electric conductor plates 1401 (emitter) and 1432 (collector), a first electricity outlet 1406 connected with the first electric conductor plate 1401, an second electricity outlet 1477 connected with the second electric conductor plate 1432, a capacitor 1461 that is connected in between the two electricity outlets 1406 and 1477, and an Earth ground 1410 that is connected with the first electricity outlet 1406.

The isothermal electricity generator system 1400A (FIG. 21a) is similar to the prototype of FIG. 16b, except that the effective heat-conduction contact of vacuum tube wall 1450 with the edges of the two electric conductor plates 1401 and 1432 in the system 1400A allow more efficient transfer of environmental heat from the tube wall to both the emitter and collector system than the prototype of FIG. 16b. Furthermore, the use of Earth ground 1410 and capacitor 1461 with the electricity outlets 1406 and 1477 as illustrated in FIG. 21a provides more stable and better system performance for isothermal electricity generation and delivery than the prototype of FIG. 16b as well.

As shown in Table 6, the work function of Mo film is about 4.36 eV and the work function of Ag—O—Cs film can be made to be anywhere between 0.5 and 1.2 eV.

In the example with the isothermal electricity generator system 1400A, the work function of Ag—O—Cs film was selected to be 0.7 eV for use as the emitter while the work function of Mo film was 4.36 eV for use as the collector as illustrated in FIG. 21a. Accordingly, when the isothermal electricity is delivered through the outlet terminals 1406 and 1477, the steady-state operating output voltage can typically be about 3.5 V in this example. Its saturation isothermal electricity current density (at output voltage of 3.5 V) is 1.55×10⁻⁵ (A/cm²) at the standard ambient temperature of 298 K (25° C.). The characteristic pattern of the ideal isothermal electricity current density (A/cm²) as a function of operating temperature T at various output voltage V(c) for this system is also similar to that of the system with a pair of emitter work function (0.70 eV) and collector work function (4.56 eV, copper Cu(110)) presented in FIG. 17b.

FIG. 21b presents an example of a prototype for an isothermal electricity generator system 1400B that has two pairs of emitters (work function 0.7 eV) and collectors (work function 4.36 eV) installed in a vacuum tube chamber. As illustrated in FIG. 21b, the system 1400B comprises: the thin layer of low work function (0.7 eV) Ag—O—Cs film 1403 coated on the first electric conductor plate 1401 right side surface to serve as the first emitter; the first vacuum space 1404 allowing the thermal electron flow 1405 to pass through ballistically between the first pair of emitter and collector; the high work function (4.36 eV) Mo film/plate 1409 coated on the second electric conductor plate 1402 left side surface facing the first emitter to serve as the first collector; the thin layer of low work function Ag—O—Cs film 1423 coated on the second electric conductor plate 1402 right side surface to serve as the second emitter; the second vacuum space 1424 allowing the thermal electron flow 1425 to pass through ballistically between the second pair of emitter and collector; the high work function Mo film/plate 1439 coated on the third electric conductor plate 1432 left side surface facing the second emitter to serve as the terminal collector; the vacuum tube wall 1450 that is in contact with the edges of the three electric conductor plates 1401, 1402 and 1432 to allow the environmental heat to transfer from the tube wall to the electric conductor plates 1401 (emitter), 1402 (collector/emitter) and 1432 (collector); the first electricity outlet 1406 connected with the first electric conductor plate 1401; the second electricity outlet 1476 connected with the second electric conductor plate 1402; the third electricity outlet 1477 connected with the third electric conductor plate 1432; the first capacitor 1461 that is connected in between the first and second electric conductor plates 1401 and 1402; the second capacitor 1462 that is connected in between the second and third electric conductor plates 1402 and 1432; and an Earth ground 1410 that is connected with the first conductor plate 1401.

The isothermal electricity generator system 1400B (FIG. 21b) is similar to the system 1400A (FIG. 21a), except that the middle electrode plate 1402 is coated with a Mo film 1409 on its left side surface and with Ag—O—Cs film at its right side surface to simultaneously serve as the first collector and the second emitter, respectively. Consequently, this system has two pairs of emitters and collectors working in series. According to Eq. 18, when a plurality (n) of the asymmetrically gated isothermal electricity generators are used in the series, the total steady-state output voltage (Vₛₜ₍ₜₒₜₐₗ₎) is the summation of the output voltages from each of the asymmetrically gated isothermal electricity generators. Therefore, when the isothermal electricity is delivered through the outlet terminals 1406 and 1477, the total steady-state output voltage (Vₛₜ₍ₜₒₜₐₗ₎) of the system 1400B is about 2×3.5 V in this example. However, the total saturation isothermal electricity current density (at output voltage of 7 V) is still about 1.55×10⁻⁵ (A/cm²) at the standard ambient operating temperature of 298 K (25° C.).

Furthermore, this system 1400B is designed to provide an option to deliver the isothermal electricity through the outlet terminals 1476 and 1477, leaving the V(c) voltage (about 3.5 V) generated by the first pair of emitter (Ag—O—Cs film 1403) and collector (Mo film/plate 1409) to serves as a bias voltage for the second emitter (Ag—O—Cs film 1423 on the second conductor plate 1402 right side surface) to more readily emit thermal electrons towards the terminal collector (Mo film/plate 1439) of the third conductor plate 1432. Sometimes, use of this option can help better extract environmental heat energy especially when the operating environmental temperature is relatively low or when the work function of certain emitters alone may not be low enough to function effectively. When the isothermal electricity is delivered through the outlet terminals 1476 and 1477, the steady-state operating output voltage is typically about 3.5 V in this example.

FIG. 21c presents an example of a prototype for an integrated isothermal electricity generator system 1400C that comprises three pairs of emitters (work function 0.7 eV) and collectors (work function 4.36 eV) installed in a vacuum tube. As illustrated in FIG. 21c, the system 1400 comprises: a thin layer of low work function (0.7 eV) Ag—O—Cs film 1403 coated on the first electric conductor plate 1401 right side surface to serve as the first emitter; a first vacuum space 1404 allowing the thermal electron flow 1405 to pass through ballistically between the first pair of emitter and collector; a (high work function 4.36 eV) Mo film/plate 1409 coated on the second electric conductor plate 1402 left side surface facing the first emitter to serve as the first collector; a thin layer of low work function (0.7 eV) Ag—O—Cs film 1423 coated on a second electric conductor plate 1402 right side surface to serve as the second emitter; a second vacuum space 1424 allowing the thermal electron flow 1425 to pass through ballistically between the second pair of emitter and collector; a (high work function 4.36 eV) Mo film/plate 1429 coated on a third electric conductor plate 1421 left side surface facing the second emitter to serve as the second collector; a thin layer of low work function (0.7 eV) Ag—O—Cs film 1433 coated on a third electric conductor plate 1421 right side surface to serve as the third emitter; a third vacuum space 1434 allowing the thermal electron flow 1435 to pass through ballistically between the third pair of emitter and collector; a (work function 4.36 eV) Mo film/plate 1439 coated on a fourth electric conductor plate 1432 left side surface facing the third emitter to serve as the terminal collector; a vacuum tube wall 1450 that is in contact with the edges of the electric conductor plates 1401, 1402. 1421 and 1432 to allow environmental heat to transfer from the tube wall to the electric conductor plates 1401 (emitter), 1402 (collector/emitter), 1421 (collector/emitter) and 1432 (collector); a first electricity outlet 1406 connected with the first electric conductor plate 1401; a second electricity outlet 1476 connected with the second electric conductor plate 1402; a third electricity outlet 1477 connected with the fourth electric conductor plate 1432; a first capacitor 1461 that is connected in between the first and second electric conductor plates 1401 and 1402; a second capacitor 1462 that is connected in between the second and third electric conductor plates 1402 and 1421; a third capacitor 1463 that is connected in between the third electric conductor plate 1421 and the fourth electric conductor plate 1432; and an Earth ground 1410 that is connected with the first electric conductor plates 1401.

As illustrated in FIG. 21c, the isothermal electricity in this example can be delivered through outlet terminals 1406 and 1476 or 1477 depending on the specific output power needs. When the isothermal electricity is delivered through outlet terminals 1406 and 1476 across a pair of emitter and collector, the steady-state operating output voltage equals to V(c), which typically can be around 3.5 V depending on the system operating conditions including the load impedance and the difference in work function between the emitter and the collector. The saturation isothermal electricity current density (at output voltage of 7 V) is about 1.55×10⁻⁵ (A/cm²) at the standard ambient temperature of 298 K (25° C.).

When the isothermal electricity is delivered through outlet terminals 1406 and 1477 across three pairs of emitters and collectors, according to Eq. 18, the steady-state operating output voltage typically can be as high as about 10.5 V. However, the total saturation isothermal electricity current density (at output voltage of 10.5 V) remains to be about 1.55×10⁻⁵ (A/cm²) at the standard ambient temperature of 298 K (25° C.) in this example.

More importantly, when the isothermal electricity is delivered through the outlet terminals 1476 and 1477, the activity of the first emitter (1401 with Ag—O—Cs film 1403) and the first collector (Mo film/plate 1409) can be used to generate a V(c) of about 3.5 V to serves as a bias voltage for the second emitter (Ag—O—Cs film 1423) on the surface of the second conductor plate 1402. In this way, the second emitter (Ag—O—Cs film 1423) will more readily emit thermal electrons towards the second collector (Mo film/plate 1429) of the third conductor plate 1421. Subsequently, the enhanced generation of V(c) at the third collector 1429 of the third conductor plate 1421 can serve as a bias voltage for the third emitter to more readily emit thermal electrons towards the terminal collector 1439 at the fourth conductor plate 1432. Therefore, use of this special feature can help better extract environmental heat energy especially when the operating environmental temperature is relatively low or when the work function of certain emitters alone may not be entirely low enough to function effectively. When the isothermal electricity is delivered through the outlet terminals 1476 and 1477, the steady-state operating output voltage can typically be about 7 V according to Eq. 18. The total saturation isothermal electricity current density (at output voltage of 7 V) remains to be about 1.55×10⁻⁵ (A/cm²) at the standard ambient temperature of 298 K (25° C.) in this example.

According to one of the various embodiments, the system capacitance for a pair of parallel emitter and collector plates is inversely dependent on their separation distance (d). It is a preferred practice to increase the capacitance across each pair of emitter and collector by properly narrowing the space separation distance (d) between the emitter surface and the collector surface to a selected space gap size in a range from as big as 100 mm to as small as in a micrometer and/or sub-micrometer scale based on specific application and operation conditions. In this way, the need of using external capacitors may be eliminated. Furthermore, use of a narrow (micrometer and/or sub-micrometer) space gap between the emitter and the collector may also help to limit the formation of the static electron space-charge clouds in the inter electrode space for better system performance. FIG. 22 presents an example of an integrated isothermal electricity generator system 1500 that has a narrow inter electrode space gap size (separation distance d) for each of the three pairs of emitters and collectors installed in a vacuum tube chamber set up vertically. The system 1500 (FIG. 22) comprises the following components installed in a vacuum tube chamber from its top to bottom: a LWF (low work function) film 1503 coated on the first electric conductor plate 1501 bottom surface to serve as the first emitter, a first narrow space 1504 allowing thermally emitted electrons 1505 to flow through ballistically between the first pair of emitter and collector, a HWF (high work function) film 1509 coated on the second electric conductor 1502 top surface to serve as the first collector, a LWF film 1523 coated on the second electric conductor 1502 bottom surface to serve as the second emitter, a second narrow space 1524 allowing thermally emitted electrons 1525 to flow through ballistically between the second pair of emitter and collector, a HWF (high work function) film 1529 coated on the third electric conductor 1521 top surface to sever as the second collector, a LWF film 1533 coated on the third electric conductor 1521 bottom surface to serve as the third emitter, a third narrow space 1534 allowing thermally emitted electrons 1535 to flow through ballistically between the third pair of emitter and collector, a HWF (high work function) film 1539 coated on the fourth electric conductor 1532 top surface to serve as the terminal (third) collector, a first electricity outlet 1506 (+) and a Earth ground 1510 that are connected with the first electric conductor plate 1501, and the second electric outlet 1537 (−) that is connected with the fourth electric conductor 1532.

The integrated isothermal electricity generator system 1500 (FIG. 22) is similar to the system 1400C (FIG. 21c) except that only the first electric conductor plate 1501 and the terminal conductor plate 1532 are wired to provide electricity outlets 1506 and 1507. Therefore, in this example, each of the second and third electric conductor plates in between the first electric conductor plate 1501 and the terminal (fourth) conductor plate 1532 is designed to simultaneously serve as a collector on its top surface and an emitter at its bottom surface. For example, the conductor plate 1502 has a collector (HWF film 1509) on the top surface facing up to receive thermally emitted electrons 1505 from the first emitter (LWF film 1503) located above the narrow space 1504 and an emitter (LWF film 1523) on the bottom side to emit thermal electrons 1525 downwards. Meanwhile, the conductor plate 1521 has a HWF film 1529 on its top surface facing up to receive thermally emitted electrons 1525 from the second emitter (LWF film 1523) located above the narrow space 1524 and a LWF film 1533 on its bottom to emit thermal electrons 1535 downwards to the terminal collector (HWF 1539) on the terminal conductor 1532. When the isothermal electricity is delivered through outlet terminals 1506 and 1537 across three pairs of emitters and collectors, the maximum total steady-state operating output voltage typically can be about 9-12 V in this example.

According to one of the various embodiments, it is a preferred practice to use an asymmetric function-gated thermal electron power generator system in an orientation with its emitter facing down and its collector is placed at the lower position facing up so that it can utilize gravity to better collect the thermally emitted electrons from the emitter placed at a higher position as illustrated in FIG. 22. In this way, the system can utilize the gravity to help pull the emitted electrons from an emitter above down to the collector below. Although the effect of the gravitational pull may be relatively small, it can help to ensure some of the emitted electrons with nearly zero kinetic energy to travel down to the collector. After any of the emitted electrons enter the collector, their contribution to the isothermal electricity is equally good in accordance with one of the various embodiment of the present invention.

For examples, some of the emitted election may have quite limited kinetic energy that may not be sufficient to overcome the repulsion force of the collector electrode's surface electrons to immediately enter the collection electrode. The use of gravitational pull provides two effects that benefit the collection of the electrons from the emission electrode. First, it can, in some extent, help accelerate the electrons from the emitter more quickly move down into the collector. The second effect is to help localize some of these emitted electrons at (and/or near) the interface between the collector surface and the vacuum space by the use of gravitational force in this manner. As shown previously with localized protons above, use of localized electron population density can enhance the utilization of environmental heat to benefit the thermal electron power generation. For instance, since free electrons including these at the interface between the collector surface and the vacuum space can gain additional kinetic energy by absorbing infrared radiation from the environment, an enhanced concentration of localized electrons at the interface between the vacuum space and the collection electrode surface enhances the probability for localized electrons to utilize their thermal motion energy to finally enter the collector electrode. After an electron enters into the collector electrode that typically has a relatively higher work function, its contribution to the thermal electron power production is essentially certain regardless of its initial kinetic energy before or after the entry.

According to one of the various embodiments, this special energy technology process for generating useful Gibbs free energy from utilization of electron thermal motion energy associated with localized electrons has a special feature that its local electron motive force (emf) generated from its special utilization of environmental heat energy may be calculated according to the following equation:

$$Local e mf = \frac{2.3 RT}{F} {log}_{10} ( 1 + [ {e}_{L}^{-} ] / [ {e}_{B}^{-} ] ) \qquad [ 22 ]$$

Where R is the gas constant, T is the absolute temperature, F is Faraday's constant, [e_{L}⁻] is the concentration of localized electrons at the interface between the collector surface and the vacuum space, and [e_{B}⁻] is the electron concentration in the bulk vacuum space.

With this Eq. 22, it is now, for the first time, understood that this local emf is a logarithmic function of the ratio of localized electron concentration [e_{L}⁻] at the interface to the delocalized electron concentration [e_{B}⁻] in the bulk vacuum gap space. Proper application of this local emf may facilitate the entry of thermal elections gap space-collector surface interface into the collector in accordance with one of the various embodiments. For example, the use of positive-charged molecular functional group-modified collector surface and/or the use of gravitational force may bring the emitted electrons to the gap space-collector surface interface forming local emf there that may help overcome the collector surface-dipole barrier to facilitate the entry of thermal electrons into the collector for enhanced isothermal electricity production.

According to one of the various embodiments, the effect of the isothermal electricity production is additive. Depending on a given specific application and its associated operating conditions such as temperature conditions, and the properties of the barrier space such as its thickness and composition, the emitter and collector electrodes and other physical chemistry properties, the number of emitter-collector pairs that may be used per integrated system as shown in FIG. 22 for the purpose of isothermally extracting environmental heat energy to generate electricity may be selected from the group consisting of 1, 2, 3, 4, 5, 6, 7, 8, 8, 9, 10, 20, 30, 40, 50, 60, 70, 80, 90, 100, 200, 300, 500, 1000, 2000, 5000, 10,000, 100,000, 1,000,000, more and/or within a range bounded by any two of these values.

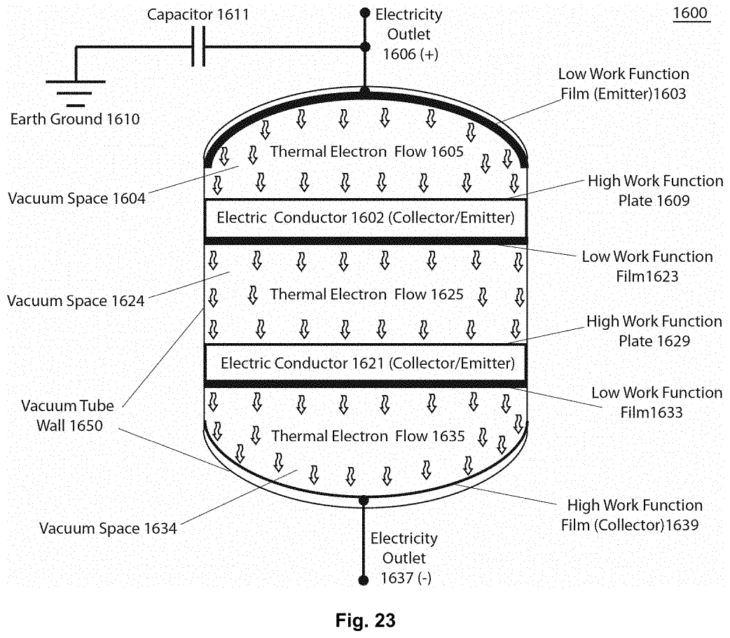

FIG. 23 presents another example of an integrated isothermal electricity generator system 1600 that has three pairs of emitters and collectors installed in a vacuum tube chamber set up vertically to utilize the gravity to help pull the emitted electrons from an emitter down to a collector. The system 1600 (FIG. 23) comprises the following components installed in a vacuum tube container from its top to bottom: a LWF (low work function) film 1603 coated onto the vacuum tube wall 1650 inner surface at the dome-shaped top end to serve as a first emitter that has an electricity outlet 1606 (+) wired with a capacitor 1611 that is connected with an Earth ground 1610, a first vacuum space 1604 allowing thermally emitted electrons 1605 to flow through ballistically, a HWF (high work function) film 1609 to serve as a first collector on the top surface of electric conductor 1602, a LWF film 1623 as the second emitter at the bottom surface of electric conductor 1602, a second vacuum space 1624 allowing thermally emitted electrons 1625 to flow through ballistically, a HWF (high work function) film 1629 as the second collector on electric conductor 1621 top surface, a LWF film 1633 as the third emitter at electric conductor 1621 bottom surface, a third vacuum space 1634 allowing thermally emitted electrons 1635 to flow through ballistically, and a HWF (high work function) film 1639 coated on the inner surface of the inversed-dome-shaped bottom end of the vacuum tube to serve as the terminal collector connected with an electricity outlet 1637 (−). When the isothermal electricity is delivered through outlet terminals 1606 and 1637 across three pairs of emitters and collectors, the maximum total steady-state operating output voltage typically can be about 9-12 V in this example.

The integrated isothermal electricity generator system 1600 (FIG. 23) is similar to the system 1500 (FIG. 22) except the following special features: 1) The system 1600 employs the inner surface of dome-shaped top end of the vacuum tube chamber as a physical support to construct the first emitter by coating an LWF (low work function) film 1603; 2) It utilizes the inner surface of the inversed-dome-shaped bottom end of the vacuum tube chamber to construct the terminal collector by coating a HWF (high work function) film 1639; and 3) the first emitter has an electricity outlet 1606 (+) wired with a capacitor 1611 that is connected with an Earth ground 1610 while the terminal collector connected with an electricity outlet 1637 (−). These features make the integrated isothermal electricity generator system 1600 much more compact than the system 1500. The optional use of capacitor 1611 between the electricity outlet 1606 (+) and the Earth ground 1610 also provides an additional way to reduce and/or modulate the possible voltage at the emitter for better system performance.

According to one of the various embodiments, during the isothermal electricity generation, an effective emitter such as those in the systems 1300, 1400, 1500 and 1600 absorbs environmental heat from the outside environment and utilizes the environmental heat energy to emit electrons as shown in FIGS. 20-22. It is important to provide effective heat conduction from the environment to the emitters. The system 1500 (FIG. 22) provide an example where the environmental heat energy primarily flow through the tube wall-electric conductor plate joints to the emitters on the electric conductor plate surfaces. Therefore, it is a preferred practice to employ heat-conductive materials in making the tube wall and more importantly the tube wall-electric conductor plate joints to ensure effective conduction of latent heat from the environment to the emitters.

The integrated isothermal electricity generator system 1600 (FIG. 23) provide an example of an emitter constructed on the inner surface of dome-shaped top end of the vacuum tube chamber by coating an LWF (low work function) film 1603. Such a close physical contact between the vacuum tube dome-shaped top wall inner surface and the emitter can favorably facilitate the heat transfer from the tube environment to the emitter.

According to one of the various embodiments, the collector surface is engineered by adding certain positively charged molecular structure such as protonated amine groups on the surface. Protonated (poly)aniline which has protonated amine groups (positive charges) on its surface made by the protonation process using the electrostatically localized excess protons as disclosed above is selected for use as a collector electrode in this embodiment.

According to one of the various embodiments, the positively charged groups such as the protonated amine groups on the collector electrode surface provide a number of beneficial effects on facilitating the collection of electrons emitted from the emitter electrode: 1) Attracting the electrons emitted from the emitter electrode, which results in an enhanced concentration of localized electron cloud [e_{L}⁻] at the vicinity of the collector electrode surface and thus enable better utilization of additional environmental heat energy according to Eq. 22 to facilitate the entry of the vacuum electrons into the collector electrode for power generation; 2) Neutralizing negative surface dipole (if any) for the collector electrode surface; and 3) Counter balancing the negative electric surface potential resulted from the accumulation of the collected electrons in the collector electrode for more power storage.

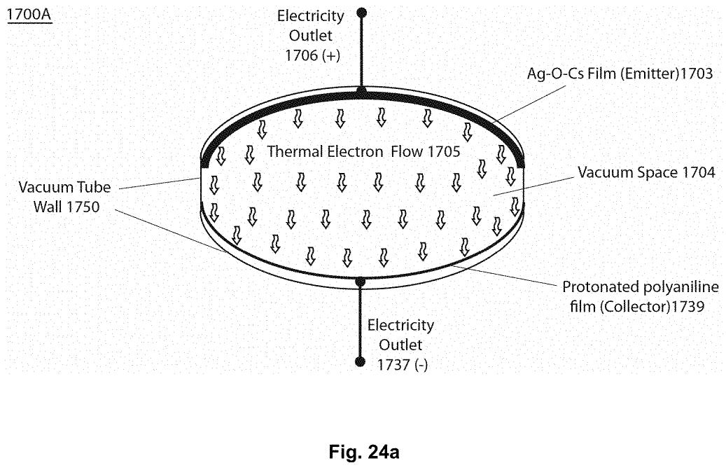

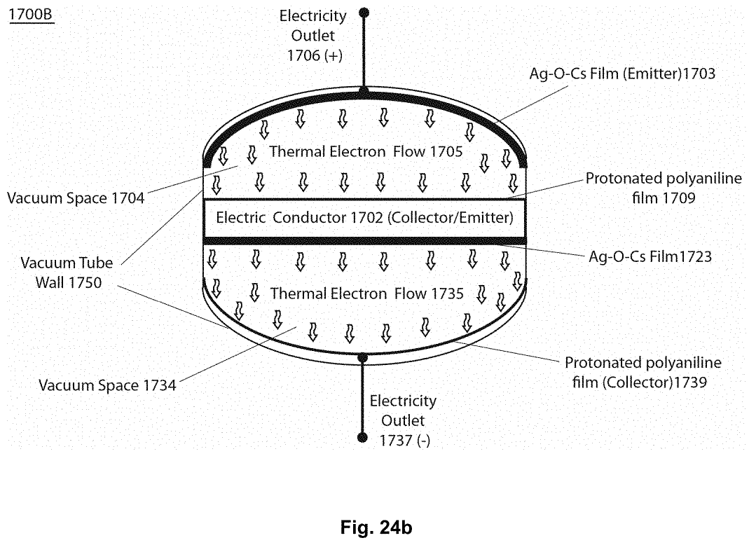

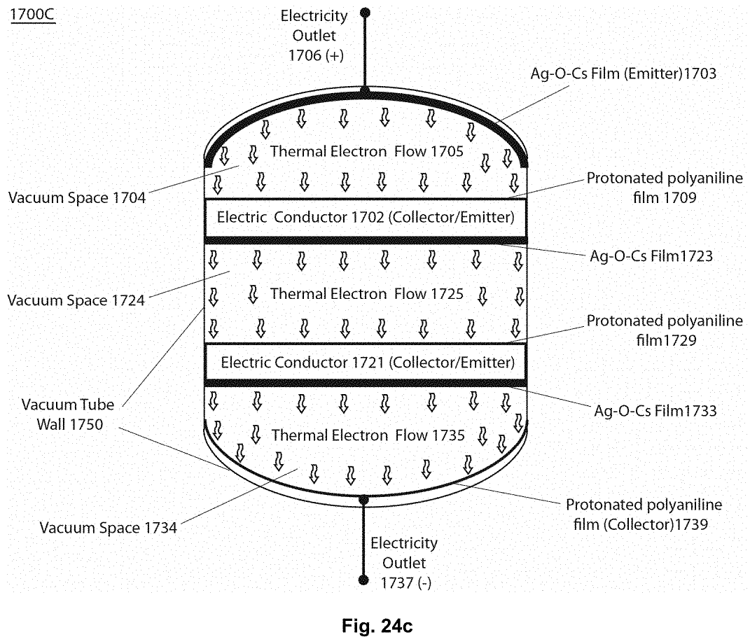

FIG. 24a presents an example of an isothermal electricity generator system 1700A that has a low work function Ag—O—Cs (0.6 eV) emitter and a high work function protonated polyaniline (4.42 eV) collector installed in a chamber-like vacuum tube with a dome-shaped top end and an inversed-dome-shaped bottom end. The system 1700A (FIG. 24a) comprises the following components installed in the chamber-like vacuum tube from its top to bottom: a Ag—O—Cs film (emitter) 1703 coated on the dome-shaped top inner surface of the chamber-like vacuum tube wall 1750 to serve as an emitter; a protonated polyaniline film 1739 coated on the inversed-dome-shaped bottom inner surface of the chamber-like vacuum tube to serve as the collector; a vacuum space 1704 allowing thermally emitted electrons 1705 to ballistically fly through between the emitter 1703 and the collector 1739; an electricity outlet 1706 (+) connected with the emitter 1703; and an electricity outlet 1737 (−) connected with the collector 1739. When the isothermal electricity is delivered through outlet terminals 1706 and 1737, the steady-state operating output voltage typically can be about 3.5 V. The saturation isothermal electricity current density (at output voltage of 3.5 V) is 7.59×10⁻⁴ A/cm² at the standard ambient temperature of 298 K (25° C.) in this example.

FIG. 24b presents an example of an integrated isothermal electricity generator system 1700B that has two pair of emitters and collectors working in series employing low work function of Ag—O—Cs (0.6 eV) and high work function of protonated polyaniline (4.42 eV). The system 1700B (FIG. 24b) comprises the following components installed in a vacuum tube chamber from its top to bottom: a Ag—O—Cs film (emitter) 1703 coated onto the inner surface of dome-shaped top end of the vacuum tube wall 1750 to serve as first emitter that has an electricity outlet 1706 (+), a vacuum space 1704 allowing thermally emitted electrons 1705 to flow through ballistically, a protonated polyaniline film 1709 to serve as the first collector on the top surface of the middle electric conductor 1702, a Ag—O—Cs film 1723 as the second emitter at the bottom surface of the middle electric conductor 1702, a second vacuum space 1734 allowing thermally emitted electrons 1735 to flow through ballistically, a protonated polyaniline film 1739 coated on the inner surface of the inversed-dome-shaped bottom end of the vacuum tube to serve as the terminal collector connected with an electricity outlet 1737 (−). When the isothermal electricity is delivered through outlet terminals 1706 and 1737, the steady-state operating output voltage typically can be about 7 V according to Eq. 18. The saturation isothermal electricity current density (at output voltage of 7 V) is about 7.59×10⁻⁴ A/cm² at the standard ambient temperature of 298 K (25° C.) in this example.

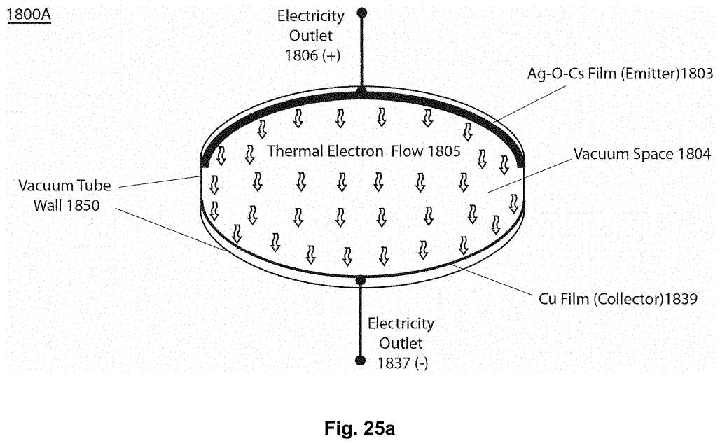

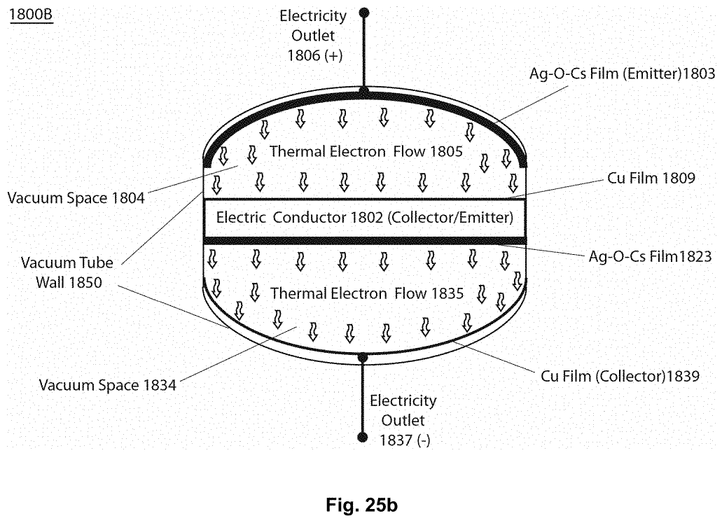

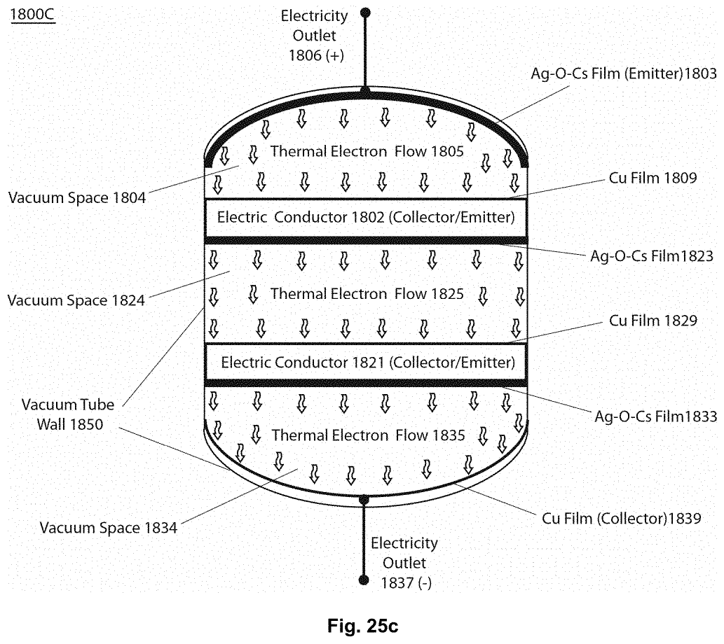

FIG. 24c presents an example of an integrated isothermal electricity generator system 1700C that has three pairs of low work function of Ag—O—Cs (0.6 eV) emitters and high work function protonated polyaniline (4.42 eV) collectors operating in series. The system 1700C (FIG. 25c) comprises the following components installed in a vacuum tube chamber from its top to bottom: a Ag—O—Cs film (emitter) 1703 coated onto the dome-shaped top end inner surface of the vacuum tube wall 1750 to serve as the first emitter; a protonated polyaniline film 1709 (collector) coated on the first middle electric conductor 1702 top surface to serve as the first collector; the first vacuum space 1704 allowing thermally emitted electrons 1705 to fly through ballistically across the first emitter and the first collector; a Ag—O—Cs film 1723 at the first middle electric conductor 1702 bottom surface to serve as the second emitter; a protonated polyaniline film 1729 coated on the second middle electric conductor 1721 top surface to serve as the second collector; the second vacuum space 1724 allowing thermally emitted electrons 1725 to fly through ballistically between the second emitter and the second collector; a Ag—O—Cs film 1733 coated on the second middle electric conductor 1721 bottom surface to serve as the third emitter, a protonated polyaniline film 1739 coated on the inversed-dome-shaped bottom end inner surface of the vacuum tube to serve as the third (terminal) collector; the third vacuum space 1734 allowing thermally emitted electrons 1735 to fly through ballistically between the third emitter and the terminal collector; the first electricity outlet 1706 (+) connected with the first emitter 1703; and the second electricity outlet 1737 (−) connected with the third (terminal) collector. When the isothermal electricity is delivered through outlet terminals 1706 and 1737 across three pairs of emitters and collectors, the maximum total steady-state operating output voltage typically can be about 10.5 V according to Eq. 18. The saturation isothermal electricity current density (at output voltage of 10.5 V) is about 7.59×10⁻⁴ A/cm² at the standard ambient temperature of 298 K (25° C.) in this example.

According to one of the various embodiments, an isothermal electrons-based environmental heat energy utilization system comprises low work function of Ag—O—Cs and high work function of Cu metal. FIG. 25a presents another example of an isothermal electricity generator system 1800A that has a low work function (0.7 eV) Ag—O—Cs emitter and a high work function (4.56 eV) Cu metal collector installed in a chamber-like vacuum tube. The system 1800A (FIG. 25a) comprises the following components installed in the chamber-like vacuum tube from its top to bottom: a Ag—O—Cs film (emitter) 1803 coated on the dome-shaped top end inner surface of the chamber-like vacuum tube wall 1850 to serve as the emitter; a vacuum space 1804 allowing thermally emitted electrons 1805 to flow through ballistically between the emitter 1803 and collector 1839; a Cu film/plate 1839 coated on the inversed-dome-shaped bottom end inner surface of the chamber-like vacuum tube to serve as the collector 1839; the first electricity outlet 1806 (+) connected with the emitter 1803; and the second electricity outlet 1837 (−) connected with the collector 1839. When the isothermal electricity is delivered through outlet terminals 1806 and 1837 across three pairs of emitters and collectors, the maximum total steady-state operating output voltage typically can be about 3.5 V. The saturation isothermal electricity current density (at output voltage of 3.5 V) is about 1.55×10⁻⁵ (A/cm²) at the standard ambient temperature of 298 K (25° C.) in this example.

FIG. 25b presents another example of an integrated isothermal electricity generator system 1800B that has two pairs of low work function Ag—O—Cs (0.7 eV) emitters and high work function Cu metal (4.56 eV) collectors operating in series. The system 1800B (FIG. 25b) comprises the following components installed in a vacuum tube chamber from its top to bottom: an Ag—O—Cs film (emitter) 1803 coated on the dome-shaped top end inner surface of the vacuum tube chamber wall 1850 to serve as the first emitter; a first vacuum space 1804 allowing thermally emitted electrons 1805 to flow through ballistically across the first pair of emitter and collector; a Cu film/plate 1809 coated on the middle electric conductor 1802 top surface to serve as the first collector; an Ag—O—Cs film 1823 coated on the middle electric conductor 1802 bottom surface to serve as the second emitter; a second vacuum space 1834 allowing thermally emitted electrons 1835 to flow through ballistically across the second pair of emitter 1823 and collector 1839; a Cu film/plate 1839 coated on the inversed-dome-shaped bottom end inner surface of the vacuum tube chamber to serve as the terminal collector; a first electricity outlet 1806 (+) connected with the first emitter 1803; and a second electricity outlet 1837 (−) connected with the terminal collector 1839.

When the isothermal electricity is delivered through outlet terminals 1806 and 1837 across two pairs of emitters and collectors, the maximum total steady-state operating output voltage of the system 1800B (FIG. 25b) typically can be about 7 V. The total saturation isothermal electricity current density (at output voltage of 7 V) is about 1.55×10⁻⁵ (A/cm²) at the standard ambient temperature of 298 K (25° C.) in this example.

FIG. 25c presents another example of an integrated isothermal electricity generator system 1800C that has three pairs of emitters and collectors operating in series employing low work function of Ag—O—Cs (0.7 eV) and high work function of Cu metal (4.56 eV). The system 1800C (FIG. 25c) comprises the following components installed in a vacuum tube from its top to bottom: a Ag—O—Cs film (emitter) 1803 coated onto the inner surface of dome-shaped top end of the vacuum tube wall 1850 to serve as the first emitter that has an electricity outlet 1806 (+), a first vacuum space 1804 allowing thermally emitted electrons 1805 to flow through ballistically, a Cu film/plate 1809 to serve as the first collector on the top surface of electric conductor 1802, a Ag—O—Cs film 1823 as the second emitter at the bottom surface of electric conductor 1802, a second vacuum space 1824 allowing thermally emitted electrons 1825 to flow through ballistically, a Cu film/plate 1829 as the second collector on electric conductor 1821 top surface, a Ag—O—Cs film 1833 as the third emitter at electric conductor 1821 bottom surface, a third vacuum space 1834 allowing thermally emitted electrons 1835 to flow through ballistically, and a Cu film/plate 1839 coated on the inner surface of the inversed-dome-shaped bottom end of the vacuum tube to serve as the terminal collector connected with an electricity outlet 1837 (−). When the isothermal electricity is delivered through outlet terminals 1806 and 1837 across three pairs of emitters and collectors, the maximum total steady-state operating output voltage typically is about 10.5 V. The total saturation isothermal electricity current density (at output voltage of 10.5 V) is about 1.55×10⁻⁵ (A/cm²) at the standard ambient temperature of 298K (25° C.) in this example.

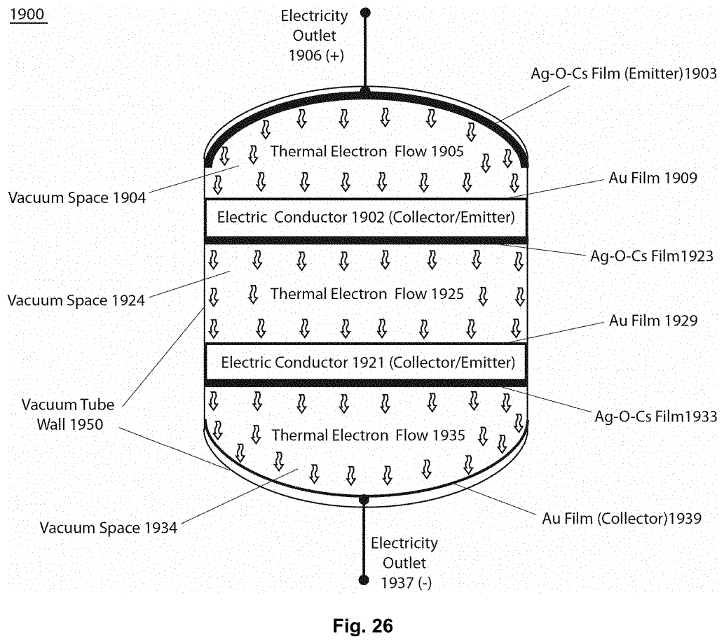

According to one of the various embodiments, an isothermal electrons-based environmental heat energy utilization system comprises low work function of Ag—O—Cs and high work function of Au metal. FIG. 26 presents another example of an integrated isothermal electricity generator system 1900 that employs three pairs of exceptionally low work function Ag—O—Cs (0.5 eV) emitters and high work function Au metal (5.10 eV) collectors working in series. The system 1900 (FIG. 26) comprises the following components installed in a vacuum tube chamber from its top to bottom: an Ag—O—Cs film (emitter) 1903 coated on the dome-shaped top end inner surface of the vacuum tube chamber wall 1950 to serve as the first emitter that has an electricity outlet 1906 (+); a first vacuum space 1904 allowing thermally emitted electrons 1905 to flow through ballistically across the first pair of emitter 1903 and collector 1909; an Au film 1909 coated on the first middle electric conductor 1902 top surface to serve as the first collector; an Ag—O—Cs film 1923 coated on the first middle electric conductor 1902 bottom surface to serve as the second emitter; a second vacuum space 1924 allowing thermally emitted electrons 1925 to flow through ballistically across the second pair of emitter 1923 and collector 1929; an Au film 1929 coated on the second middle electric conductor 1921 top surface to serve as the second collector; an Ag—O—Cs film 1933 coated on the second middle electric conductor 1921 bottom surface to serve as the third emitter; a third vacuum space 1934 allowing thermally emitted electrons 1935 to flow through ballistically across the third pair of emitter 1933 and collector 1939; and an Au film 1939 coated on the inversed-dome-shaped bottom end inner surface of the vacuum tube chamber to serve as the terminal collector connected with an electricity outlet 1937 (−). When the isothermal electricity is delivered through outlet terminals 1906 and 1937 across three pairs of emitters and collectors, the maximum total steady-state operating output voltage typically can be about 12 V. The total saturation isothermal electricity current density (at output voltage of 12 V) is about 3.73×10⁻² A/cm² at the standard ambient temperature of 298 K (25° C.) in this example.

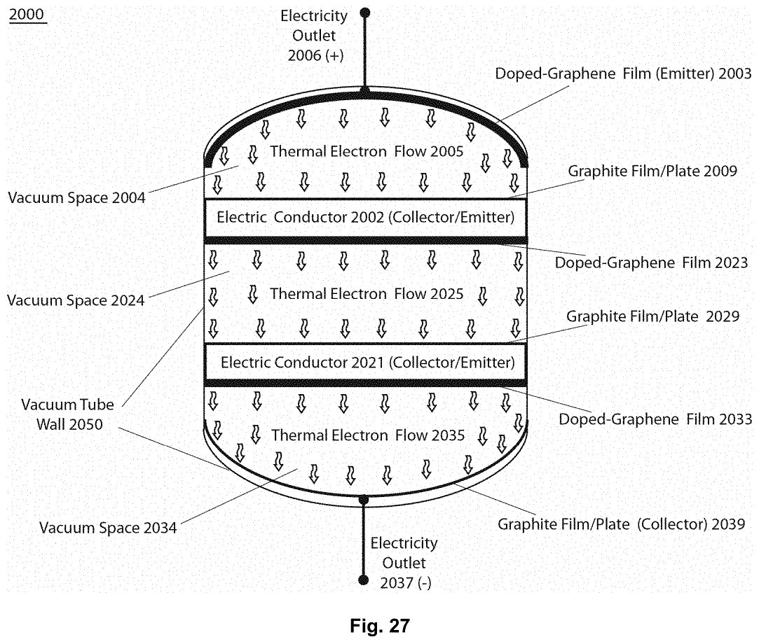

According to one of the various embodiments, an isothermal electrons-based environmental heat energy utilization system comprises low work function of doped-graphene and high work function of graphite. FIG. 27 presents another example of an integrated isothermal electricity generator system 2000 that employs low work function of doped-graphene (1.01 eV) and high work function of graphite (4.60 eV). The system 2000 (FIG. 27) comprises the following components installed in a vacuum tube from its top to bottom: a doped-graphene film (emitter) 2003 coated onto the inner surface of dome-shaped top end of the vacuum tube wall 2050 to serve as the first emitter that has an electricity outlet 2006 (+), a first vacuum space 2004 allowing thermally emitted electrons 2005 to flow through ballistically, a graphite film 2009 to serve as a collector on the top surface of the first middle electric conductor 2002, a doped-graphene film 2023 as the second emitter at the bottom surface of the first middle electric conductor 2002, a second vacuum space 2024 allowing thermally emitted electrons 2025 to flow through ballistically, a graphite film 2029 as the second collector on the second middle electric conductor 2021 top surface, a doped-graphene film 2033 as the third emitter at the second middle electric conductor 2021 bottom surface, a third vacuum space 2034 allowing thermally emitted electrons 2035 to flow through ballistically, and a graphite film 2039 coated on the inner surface of the inversed-dome-shaped bottom end of the vacuum tube to serve as the terminal collector connected with an electricity outlet 2037 (−). When the isothermal electricity is delivered through outlet terminals 2006 and 2037 across three pairs of emitters and collectors, the maximum total steady-state operating output voltage typically can be about 9 V. The total ideal saturation isothermal electricity current density (at output voltage of 9 V) at the following operating temperature is: 1.30×10⁻¹⁰ A/cm² at 298 K (25° C.), 5.14×10⁻⁷ A/cm² at 373 K (100° C.), 5.94×10⁻⁴ A/cm² at 473 K (200° C.), 6.31×10⁻² A/cm² at 573 K (300° C.), 1.76 A/cm² at 673 K (400° C.), 1.76 A/cm² at 673 K (400° C.), 17.3 A/cm² at 763 K (490° C.), 61.1 A/cm² at 823 K (500° C.), and 154 A/cm² at 873 K (600° C.) in this example.

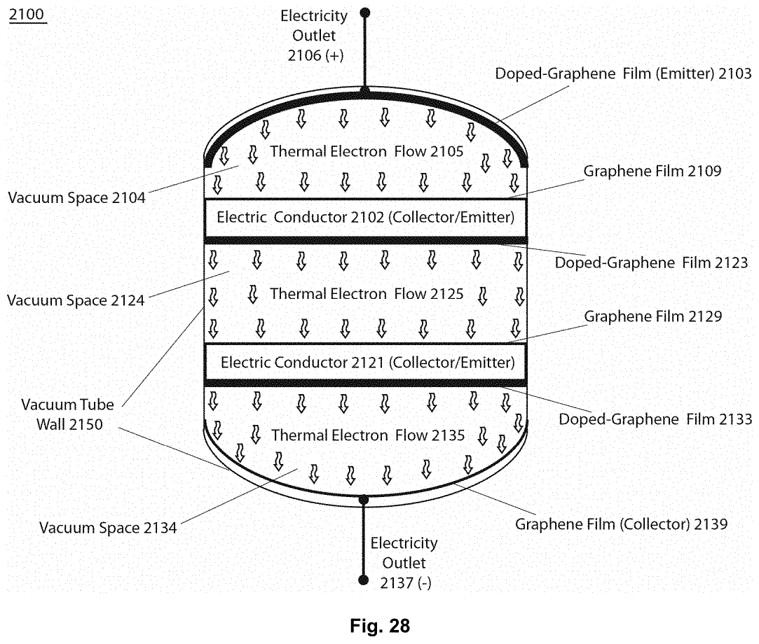

According to one of the various embodiments, an isothermal electrons-based environmental heat energy utilization system comprises low work function of doped-graphene and high work function of graphene. FIG. 28 presents another example of an integrated isothermal electricity generator system 2100 that employs multiple pairs of low work function doped-graphene (1.01 eV) emitters and high work function graphene (4.60 eV) collectors. The system 2100 (FIG. 28) comprises the following components installed in a vacuum tube chamber from its top to bottom: a doped-graphene film (emitter) 2103 coated on the dome-shaped top end inner surface of the vacuum tube chamber wall 2150 to serve as first emitter that has an electricity outlet 2106 (+), a first vacuum space 2104 allowing thermally emitted electrons 2105 to flow through ballistically across the first pair of emitter 2103 and collector 2109, a graphene film 2109 on the first middle electric conductor 2102 top surface to serve as the first collector, a doped-graphene film 2123 coated on the first middle electric conductor 2102 bottom surface to serve as the second emitter, a second vacuum space 2124 allowing thermally emitted electrons 2125 to flow through ballistically across the second pair of emitter 2123 and collector 2129, a graphene film 2129 coated on the second middle electric conductor 2121 top surface to serve as the second collector, a doped-graphene film 2133 coated on the second middle electric conductor 2121 bottom surface as the third emitter, a third vacuum space 2134 allowing thermally emitted electrons 2135 to flow through ballistically across the third pair of emitter 2133 and collector 2139, and a graphene film 2139 coated on the inversed-dome-shaped bottom end the inner surface of the vacuum tube chamber to serve as the terminal collector connected with an electricity outlet 2137 (−). When the isothermal electricity is delivered through outlet terminals 2106 and 2137 across three pairs of emitters and collectors, the maximum total steady-state operating output voltage typically can be about 9 V in this example. The total ideal saturation isothermal electricity current density (at output voltage of 9 V) at the following operating temperature is: 1.30×10⁻¹⁰ A/cm² at 298 K (25° C.), 5.14×10⁻⁷ A/cm² at 373 K (100° C.), 5.94×10⁻⁴ A/cm² at 473 K (200° C.), 6.31×10⁻² A/cm² at 573 K (300° C.), 1.76 A/cm² at 673 K (400° C.), 1.76 A/cm² at 673 K (400° C.), 17.3 A/cm² at 763 K (490° C.), 61.1 A/cm² at 823 K (500° C.), 154 A/cm² at 873 K (600° C.), 354 A/cm² at 923 K (650° C.), and 750 A/cm² at 973 K (700° C.) in this example.

According to one of the various embodiments, any of the isothermal electricity generator systems disclosed here may be modified for various applications. For examples, a typical smart mobile phone device such as iPhone 6 consumes about 10.5 Watt-hours per day (24 hours). Use of certain isothermal electricity generator systems disclosed in this invention may enable to produce a new generation of smart mobile electronic devices that can utilize the latent (existing hidden) heat energy from the ambient temperature environment to power the devices without requiring the conventional electrical power sources. For instance, use of an asymmetric function-gated isothermal electricity generator system disclosed here with a chip size of about 40 cm² that has a 3 V isothermal electricity output of 200 mA may be sufficient to continuously power a smart mobile phone device.

According to one of the various embodiments, a highly optimized isothermal electricity generator system such as the integrated isothermal electricity generator system 1900 that employs an exceptionally low work function of Ag—O—Cs (0.5 eV) and a high work function of Au metal (5.10 eV) illustrated in FIG. 26 can be powerful enough to extract environmental heat energy from an environment as cold as −20° C. (T=253 K). Consequently, it is possible to use this type of highly optimized isothermal electricity generator system to provide novel cooling for a new type of freezers and/or refrigerators while generating isothermal electricity by isothermally extracting environmental heat energy from inside the cold icebox (the heat source). Optimization and utilization of exceptionally low work function (0.5 eV) materials such as Ag—O—Cs film as an emitter are critically important to this application in extracting environmental heat energy from the interior surface of the cold box. The collector work function material for this application does not have to be gold (Au) and other work function materials such as Cu metal film, graphene and/or graphite conductors with work function about 4.6 eV can also be used.

As presented in FIG. 19b, the isothermal electricity current density (A/cm²) curves as a function of output voltage V(c) for a pair of emitter work function of 0.50 eV and collector work function of 4.60 eV showed that this type of isothermal electricity generator system can work even at a refrigerating and/or freezing temperature of 253, 263, 273, and 277 K. The saturation level of the steady-state ideal isothermal electricity current density at an output voltage of 3.50 V is: 8.42×10⁻⁴ A/cm² at 253 K (−20° C.), 2.18×10⁻³ A/cm² at 263 K (−10° C.), 5.26×10⁻³ A/cm² at 273 K (0° C.), and 7.36×10⁻³ A/cm² at 277 K (4° C.). Consequently, the cooling power of the isothermal electricity generator defined as Watt (W) per square centimeters of the cross-section area of the emitter-collector interelectrode space in this example is estimated to be: 2.88×10⁻³ W/cm² at 253 K (−20° C.), 7.63×10⁻³ W/cm² at 263 K (−10° C.), 1.84×10⁻² W/cm² at 273 K (0° C.), and 2.58×10⁻² W/cm² at 277 K (4° C.). A typical family-size freezer/refrigerator has a height of 174 cm, a depth of 80 cm and a width of 91 cm. It has a total outside surface area of 74,068 cm². Even if only 50% of the surface area is used by an asymmetric function-gated isothermal electricity generator with a cooling power density of 2.88×10⁻³ W/cm² at 253 K (−20° C.), it maximally can deliver an electricity power of 106 W plus a novel cooling power of 106 W, which is plenty to power the entire family-size freezer/refrigerator that typically requires an electricity power of only 72.5 W to run in this example.

According to one of the various embodiments, an asymmetric function-gated optimized isothermal electricity generator system that has a pair of an exceptionally low work function Ag—O—Cs (0.5 eV) emitter and a high work function graphene (4.60 eV) collector is employed to provide the novel cooling for a new type of freezer/refrigerator without requiring any of the conventional refrigeration mechanisms of compressor, condenser, evaporator and/or radiator by isothermally extracting environmental heat energy from inside the freezer/refrigerator while generating isothermal electricity.

Furthermore, use of certain isothermal electricity generator systems according to one of the various embodiments can produce electricity by utilizing the waste heat from wide varieties of waste heat sources including (but not limited to) the waste heat from electrical devices such as computers, motor vehicles engines, air-conditioner heat exchange systems, combustion-based power plants, combustion systems, heat-based distillation systems, nuclear power plants, geothermal heat sources, solar heat, and waste heat from photovoltaic panels.

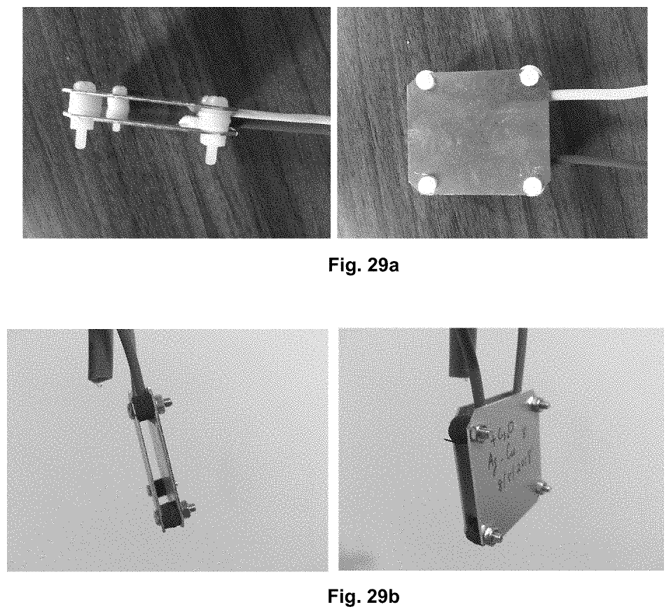

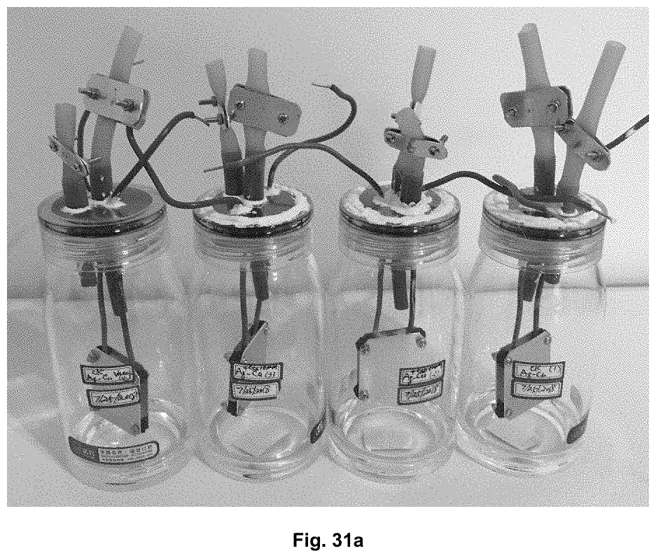

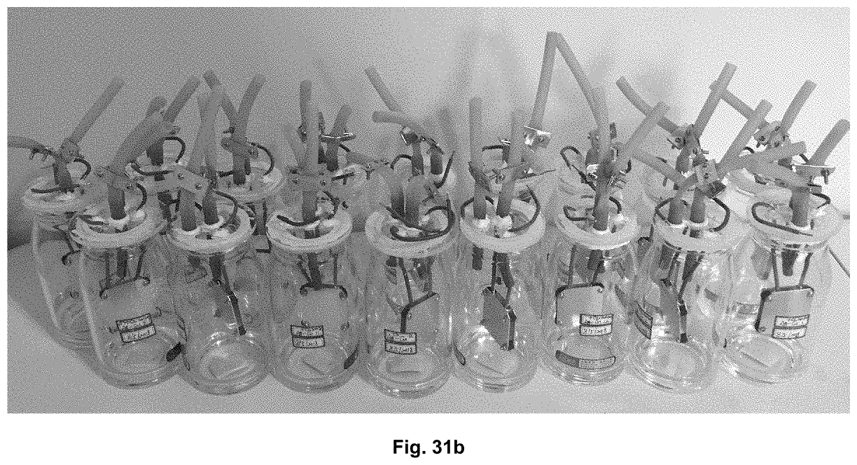

FIGS. 29-31 presents additional prototypes for an isothermal electricity generator system that comprises a pair of a low work function Ag—O—Cs emitter plate (size: 40 mm×46 mm) and a high work function Cu collector plate (size: 40 mm×46 mm) installed in a sealed glass bottle (Zhongquo Mingbei, Nuoyan Koubei, made in China) with a screw cap (FIG. 31a) or with a non-screw cap (FIG. 31b). In the electrobottle prototype design, the air inside each bottle can be readily removed though a vacuum pump to create a vacuum condition. These prototype electrobottles were made through a private effort in collaboration with a private lighting-device manufacturing company in Hangzhou City, Zhejiang Province, China.

FIG. 29a presents photographs for a pair of parallel aluminum plate-supported silver (Ag) and copper (Cu) electrode plates (size: 40 mm×46 mm) held together with electric-insulating plastic spacers (washers), screws and nuts at the four corners for each of the two electrode plates to make a pair of Ag—O—Cs type emitter (CsOAg) and Cu collector with or without oxygen plasma treatment. FIG. 29b presents photographs for a pair of parallel aluminum plate-supported silver (Ag) and copper (Cu) collector electrode plates (size: 40 mm×46 mm) held together with electric-insulating plastic spacers (washers), heat-shrink plastic tube-insulated metal screws and nuts at the corners of the electrode plates. The silver (Ag) plate and copper (Cu) collector plate were connected by soldering with a red insulator coated copper wire and a blue insulator coated copper wire, respectively. The silver (Ag) electrode plate surface was coated with a thin molecular layer of cesium oxide (Cs₂O) through painting with a dilute cesium oxide solution followed by drying to form a type of Ag—O—Cs emitter with or without oxygen plasma treatment. This shows how a pair of prototype Ag—O—Cs emitter (CsOAg) and Cu collector can be assembled.

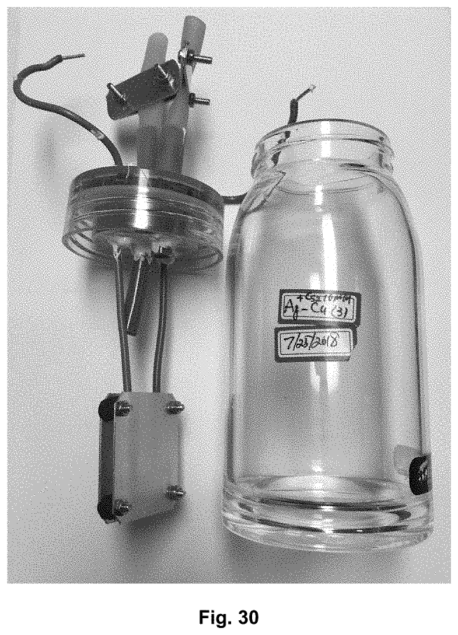

FIG. 30 presents a photograph of the parts for a prototype CsOAg—Cu electrobottle that comprise a pair of parallel aluminum plate-supported CsOAg (silver (Ag), coated with Cs₂O) and copper (Cu) collector plates installed with the red and blue insulator coated copper wires passing through a screw bottle cap. Two blue plastic air tubes were installed through two additional holes in the screw bottle cap. Electric-insulating and air-tight Kafuter 704 RTV silicone gel (white) was used to seal the joints for the wires and tubes passing through the bottle cap. This shows how a prototype CsOAg—Cu electrobottle can be assembled.

FIG. 31a presents a photograph showing four prototype CsOAg—Cu electrobottles that were fabricated using crew bottle caps. Each electrobottle comprises a pair of parallel aluminum plate-supported silver CsOAg (a type of Ag—O—Cs emitter) and copper (Cu) collector electrode surfaces installed with red and blue insulator coated wires passing through a screw bottle cap. After installation and sealing with electric-insulating and air-tight Kafuter 704 RTV silicone gel (white), air was removed from each of the electro-bottles using a vacuum pump through the blue plastic tubes with the bottle cap. FIG. 31b presents a photograph of 17 prototype CsOAg—Cu electro-bottles that were made using non-screw bottle caps and sealed with electric-insulating and air-tight Kafuter 704 RTV silicone gel (white) material.

The following methods and steps were employed in fabricating these CsOAg—Cu prototype electrobottles (FIGS. 31a and 31b): a) 1.0-mm thick aluminum sheets (size: 160 mm×184 mm with a thickness of 1.0-mm) were used as the mechanical supporting plate material; b) a pre-manufactured copper (Cu) film (35-μm thick) was mechanically pressed with a layer of 0.2-mm thick sticky heat-conductive and electric insulating gel onto an aluminum sheet (size: 160 mm×184 mm with a thickness of 1.0-mm), forming a Cu film (35-μm thick)-insulating gel (0.2-mm thick)-aluminum sheet (1-mm thick) structure; c) a 10-μm thick silver (Ag) film was then electroplated onto the Cu film (35-μm thick)-insulating gel (0.2-mm thick)-aluminum sheet (1-mm thick) structure using a sliver electroplating solution containing silver nitrate and potassium cyanide (which is highly toxic and must be carefully handled with protective equipment by fully trained professionals only), producing a 160 mm×184 mm Ag film (10-μm thick)-Cu film (35-μm thick)-insulating gel (0.2-mm thick)-aluminum sheet (1-mm thick) structure; d) a 160 mm×184 mm Cu film-insulating gel-aluminum sheet was mechanically cut to produce smaller pieces with a size of 40 mm×46 mm to serve as high work function Cu collector plates; e) similarly, a 160 mm×184 mm Ag film (10-μm thick)-Cu film (35-μm thick)-insulating gel (0.2-mm thick)-aluminum sheet (1-mm thick) structure was mechanically cut to produce smaller pieces with the size of 40 mm×46 mm to serve as Ag plates; e) the silver (Ag) electrode plate surfaces were coated with a thin molecular layer of cesium oxide (Cs₂O) through painting with a dilute (10-mM) Cs₂O solution followed by drying (alternatively, Ag plate surfaces are treated with oxygen plasma and coated with vaporized Cs atoms) to produce a type of low work function Ag—O—Cs emitter plates; f) a small hole (diameter 3 mm) was made near each of the four corners for each of the 40 mm×46 mm electrode plates using a mechanical hole maker; g) each of the Ag—O—Cs emitter plates was connected by soldering with a red insulator coated copper wire (a single 16 gauge copper wire with red insulator coat); h) similarly, each of the Cu collector plates was connected by soldering with a blue insulator coated copper wire (a single 16 gauge copper wire with blue insulator coat); i) as shown in FIG. 29b, each pair of a low work function Ag—O—Cs emitter plate (size: 40 mm×46 mm) and a high work function Cu collector plate (size: 40 mm×46 mm) was assembled in parallel with a separation distance of 5 mm using a set of four heat-shrinking plastic insulator tube-insulated metal screws, four insulating plastic washers/spacers, and four nuts (or using a set of electric-insulating plastic spacers (washers), screws and nuts as shown in FIG. 29a) at the four corners of the two electrode plates; j) as shown in FIG. 30, a pair of 3-mm-diameter holes was made in each of the bottle caps (typically made of stainless steel and/or plastic material) for the red and blue wires to pass through; k) a pair of 8-mm-diameter holes was made in the bottle cap for a pair of blue plastic (or stainless steel) tubes to pass through (to pull vacuum later); 1) the assembled pair of Ag—O—Cs emitter plate and Cu collector plate was then inserted into a glass bottle with its insulated red and blue wires passing through the 3-mm-diameter holes of the bottle cap (FIG. 30); m) all the joints around the wires and the tubes in the bottle cap were sealed with an air-tight electric-insulating Kafuter 704 RTV silicone gel material (FIGS. 30 and 31); n) after installation, air was removed from each of the electrobottles through the blue plastic tubes at the bottle cap using a vacuum pump and kept each electrobottle sealed under the vacuum condition by closing the rubber valves of the air tubes (FIG. 31); and o) quality inspection: for example, the insulation between the Ag film/Cu film and the supporting aluminum sheet by the 0.2-mm thick insulating gel and the insulation between the metal screws and the Ag film/Cu film plates by the heat-shrinking plastic insulator tubes for all metal screw bolts were inspected with electric insulation measurement for each pair of electrode plates.

Therefore, although the metal screws/nuts were in contact with the supporting aluminum sheet plates as shown in FIG. 29b, each of the CsOAg film emitter and the Cu film collector was still well insulated from both the metal screws and the supporting aluminum sheet plates. The insulator electric resistance as measured across a pair of CsOAg film emitter terminal wire (red) and Cu film collector terminal wire (blue) was over 50 MΩ for a typical CsOAg—Cu electrobottle prototype in this example.

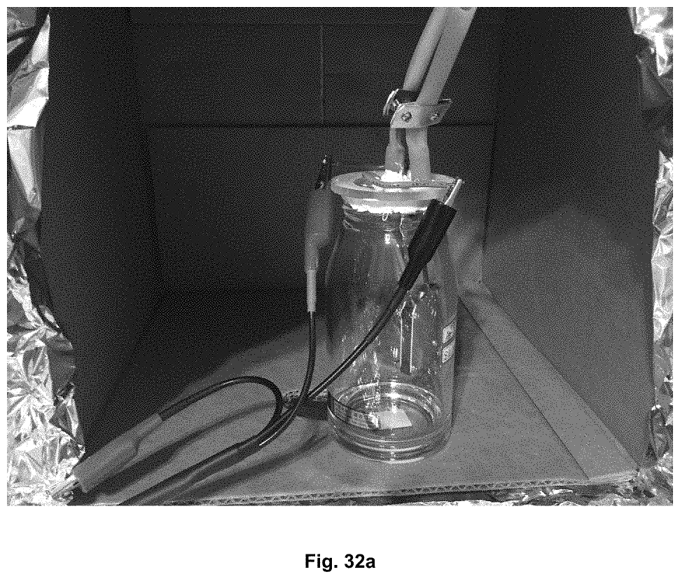

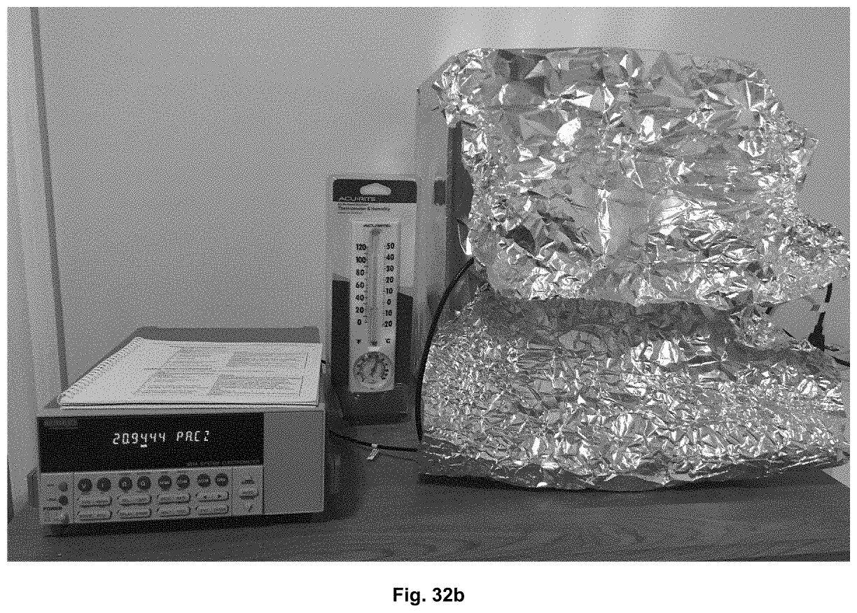

The isothermal electricity generation activity in each prototype CsOAg—Cu electrobottle was measured with a Keithley 6514 electrometer (Keithley Instruments, Inc., Cleveland, Ohio, USA) as shown in FIG. 32. During the experimental measurements, a prototype electrobottle that comprises a pair of a low work function Ag—O—Cs emitter plate (size: 40 mm×46 mm) and a high work function Cu collector plate (size: 40 mm×46 mm) installed in a sealed glass bottle was placed into a 33×30×42 cm Faraday box made of heavy duty aluminum foil to reduce the potential electric interference from the surroundings. As shown in FIG. 32a, the Keithley 6514 electrometer's red alligator clip was connected with the wire (red) of the Ag—O—Cs emitter plate while the electrometer's black alligator clip was connected to the wire (black) of the Cu collector plate. The metal Faraday box that was typically grounded by connecting with the Keithley 6514 electrometer's green alligator clip (ground wire) was closed at all sides as shown in FIG. 32b to shield the prototype electrobottle device to minimize any potential electric interference from the sounding environment during the measurements for isothermal electricity generation activity.

As shown in FIG. 32b, for example, the isothermal electricity generation was measured by a Keithley 6514 electrometer reading “20.9444 PA·CZ”. This indicates that the isothermal electric current from the prototype electrobottle device (FIG. 32a) was approximately 20.94 pico Amps (pA) as measured at a room temperature (21° C.) using the well-established Amps measurement procedure with Keithley 6514 electrometer's zero check and zero (baseline) correction (CZ) functions.

A number of prototype CsOAg—Cu electrobottles were experimentally tested for their isothermal electricity production performance. Table 10 presents examples of experimental isothermal electricity production results from a prototype isothermal electricity generator (electrobottle sample “CsOAg—Cu 1”) in comparison with a control electrobottle sample “CK Ag—Cu” as tested at 23° C. with Keithley 6514 system electrometer. The control electrobottle “CK Ag—Cu” has the same structure as that of the electrobottle “CsOAg—Cu 1” except that the Ag plate surface of the control electrobottle “CK Ag—Cu” was not coated with any cesium oxide (Cs₂O). The Amps measurement procedure with Keithley 6514 electrometer's zero check and zero (baseline) correction (CZ) was used in testing 1) the electrobottle “CsOAg—Cu 1”, 2) the Keithley 6514 system's Model 237-ALG-2 low noise cable with three alligator clips (no electrobottle device), and 3) the control electrobottle “CK Ag—Cu”. Based on the experimental measurements with 12 readings from the Keithley 6514 system electrometer, the isothermal electric current from electrobottle “CsOAg—Cu 1” was measured to be 11.17±0.08 pico amps (pA), which is well above the electrometer baseline signal of 0.071±0.17 pA as measured with Keithley 6514 system's Model 237-ALG-2 low noise cable with three alligator clips (no electrobottle device). The control electrobottle “CK Ag—Cu” gave an electric current reading of −0.360±0.005 pA, which is quite different from that (11.17±0.08 pA) of electrobottle “CsOAg—Cu 1”. Therefore, these experimental results quite clearly demonstrated the isothermal electricity production activity in the prototype electrobottle “CsOAg—Cu 1” as expected.

When the isothermal electricity from the prototype electrobottle “CsOAg—Cu 1” was measured in reverse polarity (Keithley 6514 system's Model 237-ALG-2 low noise cable black alligator connector to CsOAg plate (a type of Ag—O—Cs emitter) and red alligator connector to Cu plate), the isothermal electric current was measured to be −10.77±0.17 pA, which is quite different from that (0.220±0.003 pA) of the control electrobottle “CK Ag—Cu” when measured also in its reverse polarity (see “rev, pA·CZ” in Table 10). Therefore, these experimental results quite also clearly demonstrated the isothermal electricity production activity in the prototype electrobottle “CsOAg—Cu 1” as expected.

Table 10 presents the experimental isothermal electricity production results from a prototype electrobottle “CsOAg—Cu 1” in comparison with a control electrobottle “CK Ag—Cu” as tested at 23° C. with Keithley 6514 electrometer's zero check and zero (baseline) correction (CZ).

| | | | Cable/ | | |
|---|---|---|---|---|---|
| | | | alligator | | |
| | CsOAg—Cu 1 | CsOAg—Cu 1 | clips | CK Ag—Cu | CK Ag—Cu |
| Measurements | pA · CZ | rev, pA · CZ | pA · CZ | pA · CZ | rev, pA · CZ |
| Reading 1 | 11.11 | −10.8 | 0.071 | −0.364 | 0.222 |
| Reading 2 | 11.26 | −10.4 | 0.068 | −0.365 | 0.224 |
| Reading 3 | 11.05 | −10.62 | 0.074 | −0.365 | 0.221 |
| Reading 4 | 11.21 | −10.57 | 0.072 | −0.366 | 0.217 |
| Reading 5 | 11.14 | −10.91 | 0.073 | −0.362 | 0.213 |
| Reading 6 | 11.08 | −10.8 | 0.0725 | −0.358 | 0.216 |
| Reading 7 | 11.24 | −10.83 | 0.07 | −0.355 | 0.221 |
| Reading 8 | 11.2 | −10.97 | 0.069 | −0.350 | 0.22 |
| Reading 9 | 11.03 | −10.76 | 0.0715 | −0.354 | 0.224 |
| Reading 10 | 11.24 | −10.95 | 0.068 | −0.361 | 0.221 |
| Reading 11 | 11.21 | −10.75 | 0.0718 | −0.360 | 0.218 |
| Reading 12 | 11.27 | −10.93 | 0.0729 | −0.362 | 0.223 |
| Mean | 11.17 | −10.77 | 0.071 | −0.360 | 0.220 |
| STD | ±0.08 | ±0.17 | ±0.002 | ±0.005 | ±0.003 |
| pA/cm² | 0.607 | −0.586 | | −0.019 | 0.012 |

Note, the isothermal electron flux (J_{isoT}) normal to the surfaces of the emitter and collector (also named as the isothermal electricity current density) can be calculated as the ratio of the isothermal electric current (11.17±0.08 pA) to the CsOAg plate surface area (4.0×4.6=18.4 cm²). As listed in Table 10, the electricity current density across the CsOAg plate surface area in electrobottle “CsOAg—Cu 1” was determined to be 0.607 pA/cm² in its normal polarity and −0.586 pA/cm² when measured with its reverse polarity. By taking their absolute values, the averaged electricity current density in electrobottle “CsOAg—Cu 1” was calculated to be 0.596 pA/cm². Based on this isothermal electron flux (J_{isoT}) of 0.596 pA/cm² at 23° C., the work function of the CsOAg emitter plate surface in electrobottle “CsOAg—Cu 1” was estimated to be about 1.1 eV in this example.

Table 11 presents the experimental isothermal electricity production results from another prototype isothermal electricity generator (electrobottle “(3) CsOAg—Cu”) measured as a function of operating temperature. The standard methods of Amps and voltage measurements with Keithley 6514 electrometer's zero check and zero (baseline) correction (CZ) were used in testing this prototype “(3) CsOAg—Cu” electrobottle. Based on 12 measurement readings from Keithley 6514 system electrometer, the isothermal electric current from electrobottle “(3) CsOAg—Cu” at 20.5° C., 23° C. and 25° C. was measured to be 2.12±0.03 pA, 5.81±0.03 pA and 7.35±0.02 pA, respectively. This experimental result demonstrated that isothermal electricity production can indeed increase dramatically with the rising of environmental temperature as expected.

When the isothermal electricity from electrobottle “(3) CsOAg—Cu” was measured in reverse polarity (Keithley 6514 system's Model 237-ALG-2 low noise cable black alligator connector to CsOAg plate (a type of Ag—O—Cs emitter) and red alligator connector to Cu collector plate), the isothermal electric current was measured to be −7.43±0.03 pA (Table 11), somewhat similar to that observed in electrobottle “CsOAg—Cu 1” (Table 10).

According to the measurements with 12 readings from Keithley 6514 system electrometer, the isothermal electric voltage output from electrobottle “(3) CsOAg—Cu” at 25° C. was measured to be 54.2±0.8 mV (Table 11). Based on the isothermal electric voltage (54.2±0.8 mV) and isothermal electric current (7.35±0.02 pA) as measured at 25° C., the isothermal electricity power output was calculated to be 3.98×10⁻¹³ Watts for the electrobottle “(3) CsOAg—Cu” prototype device in this example.

As listed in Table 11, the electricity current density across the CsOAg plate surface area in electrobottle “(3) CsOAg—Cu” was measured to be 0.399 pA/cm² with normal polarity and −0.404 pA/cm² when measured with reverse polarity. By taking the absolute values, the averaged electricity current density in electrobottle “(3) CsOAg—Cu” was calculated to be 0.402 pA/cm². Based on this experimentally determined isothermal electron flux (J_{isoT}) of 0.402 pA/cm² at 25° C., the work function of the CsOAg emitter plate surface in electrobottle “(3) CsOAg—Cu” was estimated to be about 1.1 eV.

Table 11 presents the experimental isothermal electricity production results from a prototype electrobottle “(3) CsOAg—Cu” measured as a function of operating temperature at 20.5° C., 23° C. and 25° C. with Keithley 6514 electrometer's zero check and zero (baseline) correction (CZ).

| Temperature | 20.5° C. | 23° C. | 25° C. | 25° C. | 25° C. |
|---|---|---|---|---|---|
| Measurements | pA · CZ | pA · CZ | pA · CZ | rev, pA · CZ | mV · CZ |
| Reading 1 | 2.06 | 5.75 | 7.336 | −7.48 | 55.5 |
| Reading 2 | 2.10 | 5.81 | 7.361 | −7.468 | 55.2 |
| Reading 3 | 2.10 | 5.86 | 7.33 | −7.446 | 55.0 |
| Reading 4 | 2.11 | 5.80 | 7.36 | −7.455 | 54.8 |
| Reading 5 | 2.16 | 5.82 | 7.355 | −7.442 | 54.5 |
| Reading 6 | 2.14 | 5.80 | 7.342 | −7.44 | 54.3 |
| Reading 7 | 2.15 | 5.81 | 7.335 | −7.43 | 54.0 |
| Reading 8 | 2.14 | 5.82 | 7.354 | −7.415 | 53.9 |
| Reading 9 | 2.13 | 5.80 | 7.343 | −7.401 | 53.7 |
| Reading 10 | 2.10 | 5.84 | 7.35 | −7.413 | 53.5 |
| Reading 11 | 2.13 | 5.82 | 7.39 | −7.406 | 53.2 |
| Reading 12 | 2.12 | 5.84 | 7.29 | −7.418 | 53.1 |
| Mean | 2.12 | 5.81 | 7.35 | −7.43 | 54.2 |
| STD | ±0.03 | ±0.03 | ±0.02 | ±0.03 | ±0.8 |
| pA/cm² | 0.115 | 0.316 | 0.399 | −0.404 | |

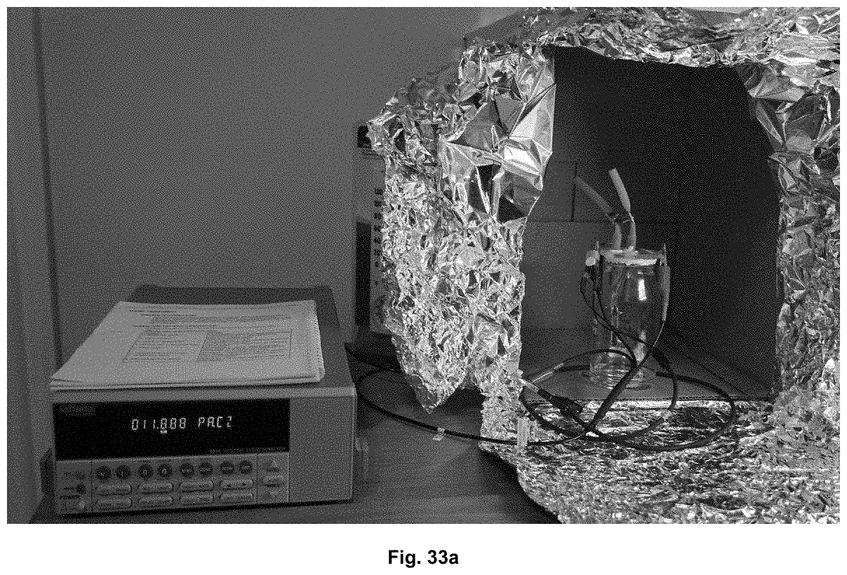

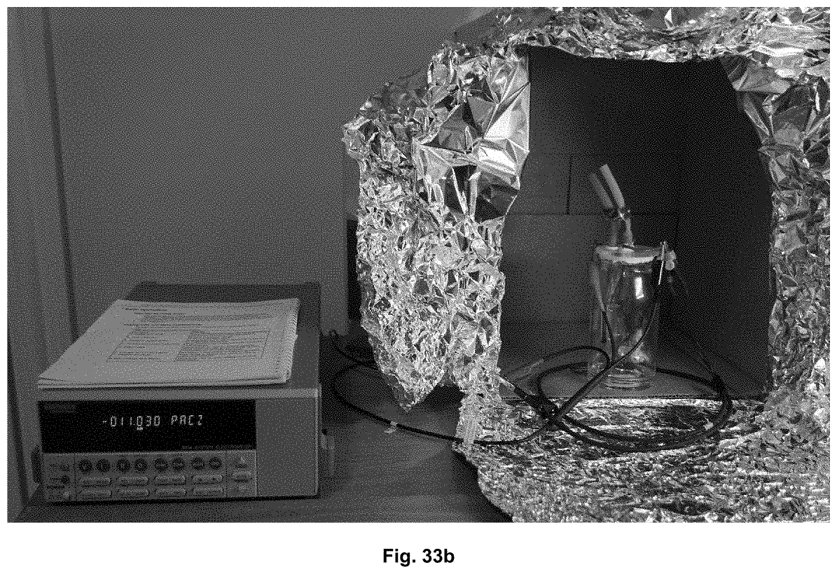

FIG. 33a presents a photograph of another prototype electrobottle placed inside a Faraday box and tested in normal polarity (Keithley 6514 system electrometer's low noise cable/red alligator connector to CsOAg plate (a type of Ag—O—Cs emitter) and black alligator connector to Cu collector plate), showing an electric current reading of “11.888 pA·CZ”. This shows that the isothermal electric current from this prototype electrobottle was approximately 11.89 pA as measured at room temperature (21° C.) with Keithley 6514 electrometer's zero check and zero (baseline) correction (CZ). When the same electrobottle was tested in its reverse polarity (Keithley 6514 black alligator connector to CsOAg plate and red alligator connector to Cu plate) as shown in FIG. 33b, it showed a negative electric current reading of “−11.030 pA·CZ”. This is important experimental result since it demonstrated that the sign of the measured electric current was indeed dependent on the polarity of the CsOAg—Cu electrobottle as expected.

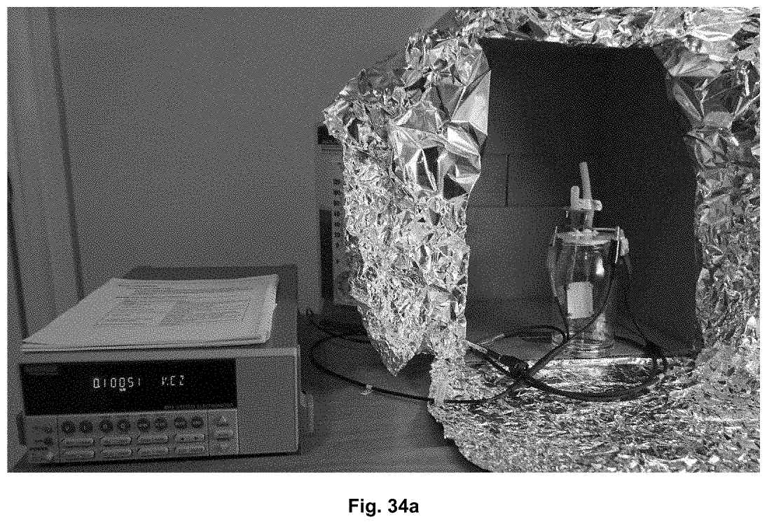

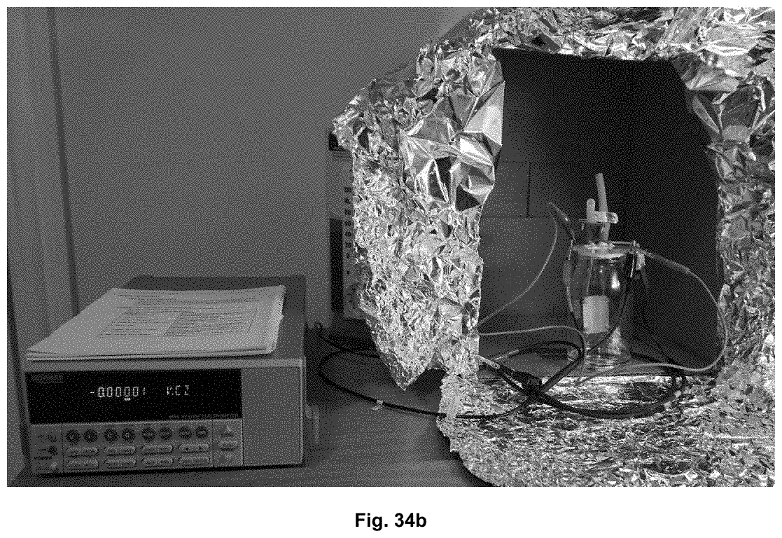

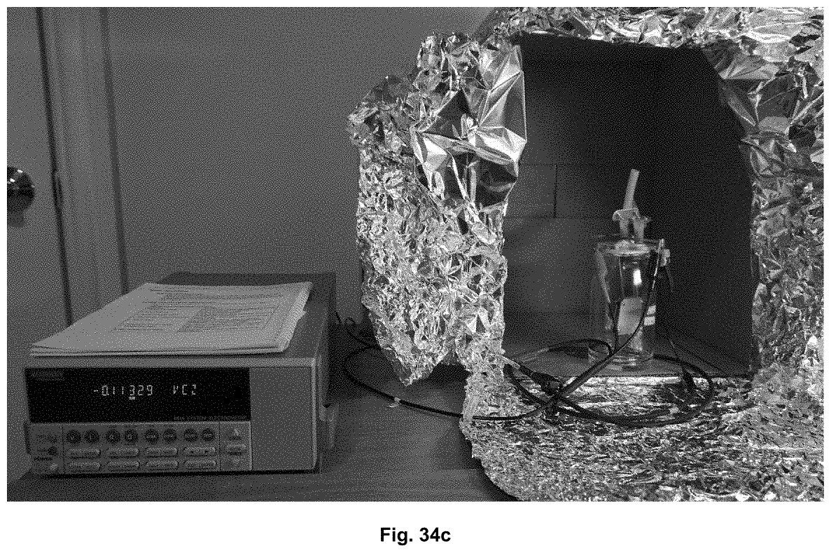

FIG. 34a presents a photograph of another CsOAg—Cu electrobottle placed inside a Faraday box and tested in normal polarity (Keithley 6514 red alligator connector to CsOAg emitter and black alligator connector to Cu collector), showing an electric voltage reading of “0.10051 V·CZ”. This shows that the isothermal electric voltage from this sample electrobottle was approximately 100.5 mV as measured at room temperature (21° C.) with Keithley 6514 electrometer's zero check and zero (baseline) correction (CZ). Subsequently, when this CsOAg—Cu electrobottle was short-circuited by connecting with a wire between the terminal (red wire) of CsOAg plate and the terminal (blue wire) of Cu plate as shown in FIG. 34b, it immediately resulted in a zero electric voltage output reading of “−0.00001 V·CZ” as expected. Finally, when the same CsOAg—Cu electrobottle was tested in reverse polarity (Keithley 6514 system's black alligator connector to CsOAg emitter plate and red alligator connector to Cu collector plate as shown in FIG. 34c), it resulted in a negative electric voltage output reading of “−0.11329 V·CZ” as expected as well. This is also an important result since it demonstrated that the sign of the measured electric voltage was indeed dependent on the polarity of the prototype CsOAg—Cu electrobottle as expected according to one of the various embodiments in the present invention.

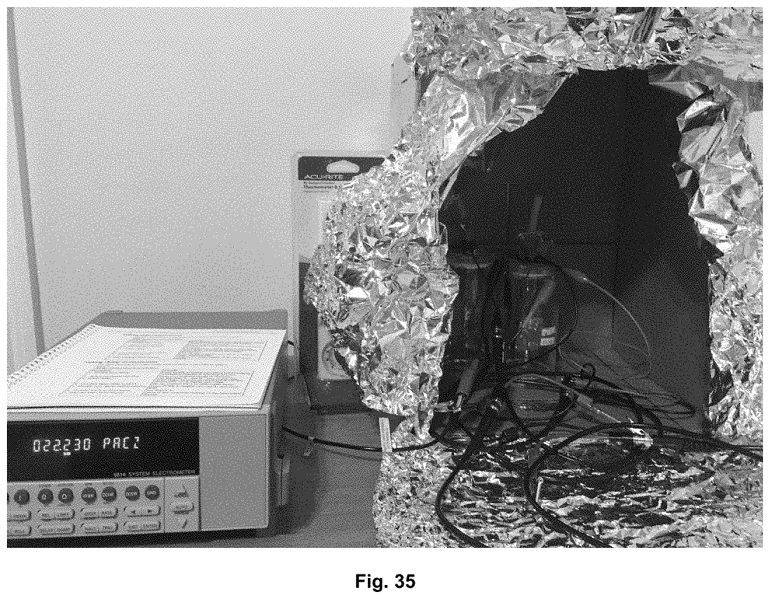

FIG. 35 presents a photograph of two prototype electrobottles connected in parallel with their normal polarity (Keithley 6514 system's red alligator connector to CsOAg emitters and black alligator connector to Cu collectors) inside a Faraday box, showing an electric current reading of “22.230 pA·CZ”. The two prototype electrobottles have an individually measured isothermal electric current of about 11 pA each. According to Eq. [20] disclosed above, when pluralities (n) of the asymmetric function-gated isothermal electricity generator systems are used in parallel, the total electrical current (Iₛₐₜ₍ₜₒₜₐₗ₎) is the summation of the electrical current (Iₛₐₜ₍ᵢ₎ as of Eq. [16]) from each of the asymmetric function-gated isothermal electricity generator systems. Therefore, the predicted isothermal electric current for the two prototype electrobottles used in parallel should be 22 pA, which excellently matched with the measured electric current reading of “22.230 pA·CZ”. This is an important result since it demonstrated that the isothermal electricity generation effects of the electrobottles are indeed additive in nature as expected in accordance with one of the various embodiments in the present invention.

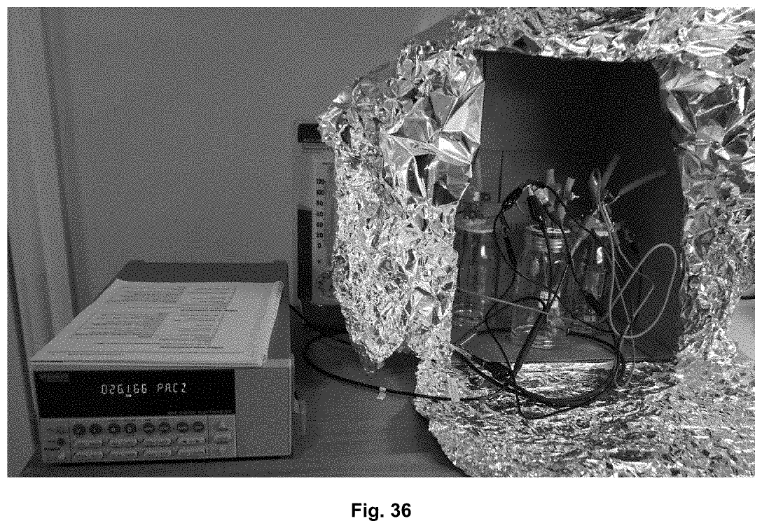

FIG. 36 presents a photograph of three prototype electrobottles connected in parallel in normal polarity (Keithley 6514 red alligator connector to CsOAg emitters and black alligator connector to Cu collectors) inside a Faraday box, showing an electric current reading of “26.166 pA·CZ”. As noted above, the first two prototype electrobottles have an individually measured isothermal electric current of about 11 pA each and the third electrobottle has an measured isothermal electric current of about 4 pA. Therefore, the predicted total isothermal electric current for the three prototype electrobottles connected in parallel should be 26 pA, which matched well with the measured electric current reading of “26.166 pA·CZ”. This is an important result since it again demonstrated that the isothermal electricity generation effects of the prototype electrobottle devices are indeed additive in accordance with one of the various embodiments in the present invention.

While the present invention has been illustrated by description of several embodiments and while the illustrative embodiments have been described in considerable detail, it is not the intention of the applicant to restrict or in any way limit the scope of the invention claims to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of applicant's general inventive concept.

What is claimed is:

1. An energy renewal method for generating isothermal electricity with making and using a special asymmetric function-gated isothermal electricity power generator system comprising at least one pair of a low work function thermal electron emitter and a high work function electron collector across a barrier space installed in a container with electric conductor support to enable a series of energy recycle process functions with utilization of environmental heat energy isothermally for at least one of:
a) utilization of environmental heat energy for energy recycling and renewing of fully dissipated waste heat energy from the environment to generate electricity with an output voltage and electric current to do useful work;
b) providing a novel cooling function for a new type of refrigerator without requiring any of the conventional refrigeration mechanisms of compressor, condenser, evaporator and radiator by isothermally extracting environmental heat energy from inside the refrigerator while generating isothermal electricity; and
c) combinations thereof.

2. The method according to claim 1, wherein the special asymmetric function-gated isothermal electron-based power generator system is an integrated isothermal electricity generator system that has a narrow inter electrode space gap size for each pair of emitter and collector installed in a vacuum tube chamber set up vertically comprising:
a low work function film coated on the first electric conductor plate bottom surface to serve as the first emitter;
a first narrow space allowing thermally emitted electrons to flow through ballistically between the first pair of emitter and collector;
a high work function film coated on the second electric conductor top surface to serve as the first collector;
a low work function film on the second electric conductor bottom surface to serve as the second emitter;
a second narrow space allowing thermally emitted electrons to flow through ballistically between the second pair of emitter and collector;
a high work function film coated on the third electric conductor top surface to sever as a second collector;
a low work function film coated on the third electric conductor bottom surface to serve as the third emitter;
a third narrow space allowing thermally emitted electrons to flow through ballistically between the third pair of emitter and collector;
a high work function film coated on the fourth electric conductor top surface to serve as the terminal collector,
a first electricity outlet and an Earth ground that are connected with the first electric conductor plate;
and a second electric outlet that is connected with the fourth electric conductor.

3. The method according to claim 2, wherein the inter electrode space gap size is selected from the group consisting of: 2 nm, 3 nm, 4 nm, 5 nm, 6 nm. 7, nm, 8 nm, 9 nm, 10 nm, 12 nm, 14 nm, 16 nm, 18 nm, 20 nm, 25 nm, 30 nm, 35 nm, 40 nm 45 nm, 50 nm, 60 nm, 70 nm, 80 nm, 100 nm, 120 nm, 140 nm 160 nm, 180 nm, 200 nm, 250 nm, 300 nm, 500 nm, 600 nm, 700 nm, 800 nm, 900 nm, 1000 nm, 1.2 μm, 1.4 μm, 1.6 μm, 1.8 μm, 2.0 μm, 2.5 μm, 3.0 μm, 3.5 μm, 4.0 μm, 4.5 μm, 5.0 μm, 6.0 μm, 7.0 μm, 9.0 μm, 10 μm, 12 μm, 14 μm, 16 μm, 18 μm, 20 μm, 25 μm, 30 μm, 35 μm, 40 μm, 45 μm, 50 μm, 60 μm, 70 μm, 80 μm, 90 μm, 100 μm, 120 μm, 140 μm, 160 μm, 180 μm, 200 μm, 250 μm, 300 μm, 400 μm, 500 μm, 600 μm, 700 μm, 800 μm, 900 μm, 1000 μm, 1.2 mm, 1.4 mm, 1.6 mm, 1.8 mm, 2.0 mm, 2.5 mm, 3.0 mm, 4.0 mm, 5.0 mm, 6.0 mm, 7.0 mm, 8.0 mm, 9.0 mm, 10 mm, 12 mm, 15 mm, 20 mm, 30 mm, 40 mm, 50 mm, 60 mm, 80 mm, 100 mm and/or within a range bounded by any two of these values.

4. The method according to claim 1, wherein the special asymmetric function-gated isothermal electron-based power generator system is an isothermal electricity generator system that has a low work function Ag—O—Cs (0.6 eV) emitter and a high work function protonated polyaniline (4.42 eV) collector installed in a chamber-like vacuum tube comprising:
an Ag—O—Cs film coated on the dome-shaped top inner surface of the chamber-like vacuum tube wall to serve as an emitter;
a protonated polyaniline film coated on the inversed-dome-shaped bottom inner surface of the chamber-like vacuum tube to serve as the collector;
a vacuum space allowing thermally emitted electrons to ballistically fly through between the emitter and the collector;
an electricity outlet connected with the emitter;
and an electricity outlet connected with the collector.

5. The method according to claim 1, wherein the special asymmetric function-gated isothermal electron-based power generator system is an integrated isothermal electricity generator system that has three pairs of low work function of Ag—O—Cs (0.6 eV) emitters and high work function protonated polyaniline (4.42 eV) collectors operating in series comprising:
an Ag—O—Cs film coated on the dome-shaped top inner surface of the vacuum tube wall to serve as the first emitter;
a protonated polyaniline film (collector) coated on the first middle electric conductor top surface to serve as the first collector;
a first vacuum space allowing thermally emitted electrons to fly through ballistically across the first emitter and the first collector;
an Ag—O—Cs film coated on the first middle electric conductor bottom surface to serve as the second emitter;
a protonated polyaniline film coated on the second middle electric conductor top surface to serve as the second collector;
a second vacuum space allowing thermally emitted electrons to fly through ballistically between the second emitter and the second collector;
an Ag—O—Cs film coated on the second middle electric conductor bottom surface to serve as the third emitter,
a protonated polyaniline film coated on the inversed-dome-shaped bottom inner surface of the vacuum tube to serve as the third collector;
a third vacuum space allowing thermally emitted electrons to fly through ballistically between the third emitter and the third collector;
a first electricity outlet connected with the first emitter;
and a second electricity outlet connected with the terminal collector.

6. The method according to claim 1, wherein the special asymmetric function-gated isothermal electron-based power generator system is an isothermal electricity generator system that has a low work function (0.7 eV) Ag—O—Cs emitter and a high work function Cu metal (4.56 eV) collector installed in a chamber-like vacuum tube comprising:
an Ag—O—Cs film coated on the dome-shaped top end inner surface of the chamber-like vacuum tube wall to serve as the emitter;
a vacuum space allowing thermally emitted electrons to flow through ballistically between the emitter and collector;
a Cu film coated on the inversed-dome-shaped bottom end inner surface of the chamber-like vacuum tube to serve as the collector;
a first electricity outlet connected with the emitter;
and a second electricity outlet connected with the collector.

7. The method according to claim 1, wherein the special asymmetric function-gated isothermal electron-based power generator system is an integrated isothermal electricity generator system that has two pairs of low work function Ag—O—Cs (0.7 eV) emitters and high work function Cu metal (4.56 eV) collectors operating in series comprising:
an Ag—O—Cs film coated on the dome-shaped top end inner surface of the vacuum tube chamber wall to serve as the first emitter;
a first vacuum space allowing thermally emitted electrons to flow through ballistically across the first pair of emitter and collector;
a Cu film/plate coated on the middle electric conductor top surface to serve as the first collector;
an Ag—O—Cs film coated on the middle electric conductor bottom surface to serve as the second emitter,
a second vacuum space allowing thermally emitted electrons to flow through ballistically across the second pair of emitter and collector;
a Cu film coated on the inversed-dome-shaped bottom end inner surface of the vacuum tube chamber to serve as the terminal collector;
a first electricity outlet connected with the first emitter;
and a second electricity outlet connected with the terminal collector:

8. The method according to claim 1, wherein the special asymmetric function-gated isothermal electron-based power generator system is an integrated isothermal electricity generator system that employs three pairs of exceptionally low work function Ag—O—Cs (0.5 eV) emitters and high work function Au metal (5.10 eV) collectors working in series comprising:
an Ag—O—Cs film coated on the dome-shaped top end inner surface of the vacuum tube chamber wall to serve as first emitter that has an electricity outlet;
a first vacuum space allowing thermally emitted electrons to flow through ballistically across the first pair of emitter and collector;
an Au film coated on the first middle electric conductor top surface to serve as the first collector;
an Ag—O—Cs film coated on the first middle electric conductor bottom surface to serve as the second emitter;
a second vacuum space allowing thermally emitted electrons to flow through ballistically across the second pair of emitter and collector;
an Au film coated on the second middle electric conductor top surface to serve as the second collector;
an Ag—O—Cs film coated on the second middle electric conductor bottom surface as the third emitter;
a third vacuum space allowing thermally emitted electrons to flow through ballistically across the third pair of emitter and collector;
and an Au film coated on the inversed-dome-shaped bottom end inner surface of the vacuum tube chamber to serve as the terminal collector connected with an electricity outlet.

9. The method according to claim 1, wherein the special asymmetric function-gated isothermal electron-based power generator system is an integrated isothermal electricity generator system that employs multiple pairs of low work function doped-graphene (1.01 eV) emitters and high work function graphene (4.60 eV) collectors comprising:
a doped-graphene film coated on the dome-shaped top end inner surface of the vacuum tube chamber wall to serve as first emitter that has an electricity outlet;
a first vacuum space allowing thermally emitted electrons to flow through ballistically across the first pair of emitter and collector;
a graphene film coated on the first middle electric conductor top surface to serve as the first collector,
a doped-graphene film coated on the first middle electric conductor bottom surface to serve as the second emitter;
a second vacuum space allowing thermally emitted electrons to flow through ballistically across the second pair of emitter and collector,
a graphene film coated on the second middle electric conductor top surface to serve as the second collector;
a doped-graphene film coated on the second middle electric conductor bottom surface as the third emitter;
a third vacuum space allowing thermally emitted electrons to flow through ballistically across the third pair of emitter and collector;
and a graphene film coated on the inversed-dome-shaped bottom end inner surface of the vacuum tube chamber to serve as the terminal collector connected with an electricity outlet.

10. The method according to claim 1, wherein the said low work function thermal electron emitter has a special work function value selected from the group consisting of 0.2 eV, 0.3 eV, 0.4 eV, 0.5 eV, 0.6 eV, 0.7 eV, 0.8 eV, 0.9 eV, 1.0 eV, 1.1 eV, 1.2 eV, 1.3 eV, 1.4 eV, 1.5 eV, 1.6 eV, 1.7 eV, 1.8 eV, 1.9 eV, 2.0 eV, 2.1 eV, 2.2 eV, 2.4 eV, 2.6 eV, 2.8 eV, 3.0 eV, and a range bounded by any two of these values.

11. The method according to claim 1, wherein the said high work function electron collector has a special work function value selected from the group consisting of 1.0 eV, 1.1 eV, 1.2 eV, 1.3 eV, 1.4 eV, 1.5 eV, 1.6 eV, 1.7 eV, 1.8 eV, 1.9 eV, 2.0 eV, 2.1 eV, 2.2 eV, 2.4 eV, 2.6 eV, 2.8 eV, 3.0 eV, 3.2 eV, 3.4 eV, 3.6 eV, 3.8 eV, 4.0 eV, 4.2 eV, 4.4 eV, 4.6 eV, 4.8 eV, 5.0 eV, 5.5 eV, 6.0 eV, and a range bounded by any two of these values.

12. The method according to claim 1, wherein the said asymmetric function-gated isothermal electricity power generator system is designed to isothermally operate at a temperature or temperature range selected from a group consisting of 193K (−80° C.), 200K (−73° C.), 210K (−63° C.), 220K (−53° C.), 230K (−43° C.), 240K (−33° C.), 250K (−23° C.), 260K (−13° C.), 270K (−3° C.), 273K (0° C.), 278K (5° C.), 283K (10° C.), 288K (15° C.), 293K (20° C.), 298K (25° C.), 303K (30° C.), 308K (35° C.), 313K (40° C.), 318K (45° C.), 323K (50° C.), 328K (55° C.), 333K (60° C.), 338K (65° C.), 343K (70° C.), 348K (75° C.), 353K (80° C.), 363K (90° C.), 373K (100° C.), 383K (110° C.), 393K (120° C.), 403K (130° C.), 413K (140° C.), 423K (150° C.), 433K (160° C.), 453K (180° C.), 473K (200° C.), 493K (220° C.), 513K (240° C.), 533K (260° C.), 553K (280° C.), 573K (300° C.), 623K (350° C.), 673K (400° C.), 723K (450° C.), 773K (500° C.), 823K (550° C.), 873K (600° C.), 923K (650° C.), 973K (700° C.), 1073K (800° C.), 1173K (900° C.), 1273K (1000° C.), 1373K (1100° C.), 1473K (1200° C.), and a range bounded by any two of these values.

13. The method according to claim 1, wherein the said low work function thermal electron emitter is made from special emitter material that is selected from a group consisting of Ag—O—Cs, Cs₂O-coated Ag plate surface, K—O/Si(100), C12A7:e·, K on WTe2, P-doped diamond, P-doped diamond, Ca₂₄Al₂₈O₆₄, Cs/O doped graphene, Sr₁₋ₓ, BaₓVO₃, Ba-coated SiC, O—Ba on W, Cs on Pt metal and combinations thereof.

14. The method according to claim 1, wherein the said high work function electron collector is made from special collector material that is selected from a group consisting of platinum (Pt) metal, silver (Ag) metal, gold (Au) metal, copper (Cu) metal, molybdenum (Mo) metal, aluminum (Al) metal, tungsten, rhenium, molybdenum, niobium, nickel, graphene, graphite, polyaniline film, ZnO metal oxide, ITO metal oxide, FTO metal oxide, 2-dimensional nickel, PEDOT:PSS, protonated-polyaniline film and combinations thereof.

15. The method according to claim 1, wherein the said emitter is coated on certain surface of an electric conductor that is selected from the group consisting of: heat-conducting electric conductors, heat-conducting metallic conductors, refractory metals, metal alloys, stainless steels, aluminum, copper, silver, gold, platinum, molybdenum, conductive MoO₃, tungsten, rhenium, molybdenum, niobium, nickel, titanium, graphene, graphite, heat-conducting electrically conductive polymers, polyaniline film, protonated-polyaniline film and combinations thereof.

16. The method according to claim 1, wherein the said collector is coated on certain surface of an electric conductor that is selected from the group consisting of: heat-conducting electric conductors, heat-conducting metallic conductors, refractory metals, metal alloys, stainless steels, aluminum, copper, silver, gold, platinum, molybdenum, conductive MoO₃, tungsten, rhenium, molybdenum, niobium, nickel, titanium, graphene, graphite, heat-conducting electrically conductive polymers, polyaniline film, protonated-polyaniline film and combinations thereof.

17. The method according to claim 1, wherein the said container is made with a varieties of heat-conducting wall materials that are selected from the group consisting of heat-conducting metals including stainless steels, aluminum, copper and metal alloys, vacuum-tube glass, vacuum lamp-bulb glass, electric insulating materials, carbon fibers composite materials, vinyl ester, epoxy, polyester resin, thermoplastic, highly heat-conductive graphene, graphite, cellulose nanofiber/epoxy resin nanocomposites, heat-conductive and electrical insulating plastics, heat-conductive and electrical insulating ceramics, heat-conductive and electrical insulating glass, fiberglass-reinforced plastic materials, borosilicate glass, Pyrex glass, fiberglass, sol-gel, silicone gel, silicone rubber, quartz mineral, diamond material, glass-ceramic, transparent ceramics, clear plastics, such as Acrylic (polymethyl methacrylate), Butyrate (cellulose acetate butyrate), Lexan (polycarbonate), and PETG (glycol modified polyethylene terephthalate), polypropylene, polyethylene (or polyethene) and polyethylene HD, thermally conductive transparent plastics, heat conductive and electrical insulating paint, colorless glass, clear transparent plastics containing certain anti-reflection materials or coatings, clear glass containing certain anti-reflection materials, and combinations thereof

18. The method according to claim 1, wherein the interfacing contact and seal between the said container wall and the electrode plates is made with certain heat-conductive but electrical insulating materials that are selected from the group consisting of heat-conductive and electrical insulating plastics, epoxy, polyester resin, air-tight electric-insulating Kafuter 704 RTV silicone gel material, thermoplastic, heat-conductive and electrical insulating ceramics, heat-conductive and electrical insulating glass, highly heat-conductive graphene, graphite, clear plastics, for example, Acrylic (polymethyl methacrylate), Butyrate (cellulose acetate butyrate), Lexan (polycarbonate), and PETG (glycol modified polyethylene terephthalate), polypropylene, polyethylene, and polyethylene HD, thermally conductive transparent plastics, heat conductive glues, electric insulating glues, heat conductive paint, electric insulating paint, heat conductive glass, borosilicate glass such as Pyrex glass, sol-gel, silicone gel, silicone rubber, quartz mineral, diamond material, cellulose nanofiber/epoxy resin nanocomposites, carbon fibers composite materials, glass-ceramic materials, transparent ceramics, clear transparent plastics containing anti-reflection materials and/or coating, clear glass containing anti-reflection materials and combinations thereof.

19. The method according to claim 1, wherein the asymmetric function-gated isothermal electricity power generator system with said energy recycle process functions comprises a feature where its isothermally generated electricity current density (J_{isoT}) from extraction of environmental heat energy may be calculated according to:
J_{isoT}=AT²(e^{-[WF(e)+e·V(e)]/kT}−e^{-[WF(c)+e·V(c)]/kT})
where A is the universal factor (also known as the Richardson-Dushman constant) can be expressed as 4πmek²/h³≈120 Amp/(K²·cm²) [where m is the electron mass, e is the electron unit charge, k is the Boltzmann constant and h is Planck constant]. T is the absolute temperature in Kelvin (K) for both the emitter and the collector; WF (e) is the work function of the emitter surface; the term of e·V (e) is the product of electron charge e and voltage V (e) at the emitter; k is the Boltzmann constant in (eV/K); WF (c) is the work function of collector surface; and e·V (c) is the product of electron charge e and voltage V(c) at the collector.

20. The method according to claim 1, wherein the special asymmetric function-gated isothermal electron-based power generator system that has a pair of an exceptionally low work function Ag—O—Cs (0.5 eV) emitter and a high work function graphene (4.60 eV) collector is employed to provide novel cooling for a new type of refrigerator by isothermally extracting environmental heat energy from inside the refrigerator while generating isothermal electricity.
